(19) United States
(12) Patent Application Publication
(10) Pub. No.: US 2009/0011974 A1
(43) Pub. Date: Jan. 8, 2009

(54) Scavenger Receptor B1 (Cla-1) Targeting for the Treatment of Infection, Sepsis and Inflammation

(76) Inventors: Alexander V. Bocharov, Silver Spring, MD (US); Amy L. Patterson, Rockville, MD (US); Alan T. Remaley, Bethesda, MD (US); Tatyana V. Vishnyakova, Silver Spring, MD (US); Irina N. Baranova, Bethesda, MD (US); Gyorgy Csako, Rockville, MD (US); Thomas L. Eggerman, Rockville, MD (US)

Correspondence Address: Catherine Joyce;Liniak, Berenato & White, 6550 Rock Spring Drive, Suite 240, Bethesda, MD, 20817, US

(21) Appl. No.: 10/533,103

(22) PCT Filed: Oct. 30, 2003
(86) PCT No.: PCT/US03/34511

§ 371 Date: Jun. 5, 2006

Related U.S. Application Data
(60) Provisional application No. 60/422,105, filed on Oct. 30, 2002.

(51) Int. Cl. A61K 38/02; A61P 31/00; A61P 29/00

(52) U.S. Cl. 514/2

(57) ABSTRACT

This invention relates to methods and compositions for the treatment of sepsis, inflammation or infection. In particular, the invention concerns the use of molecule(s) that target SR-BI, which is also referred to as CLA-1 (SR-BI/CLA-1), to treat sepsis, bacterial and viral infections, and inflammatory diseases. SRB I/CLA-1 ligands contributing to the pathogenesis of disease include LPS, LTA, viral envelope proteins, beta-amyloid, serum Amyloid A and/or heat shock proteins.

### CROSS-REFERENCE TO RELATED APPLICATION

This application claims priority to U.S. Patent Application Ser. No. 60/422,105, filed Oct. 30, 2002, herein incorporated by reference in its entirety.

### STATEMENT OF GOVERNMENTAL INTEREST

This invention was funded by the National Heart, Lung and Blood Institute, the W.G. Magnuson Clinical Center and the National Institute of Diabetes and Digestive and Kidney Diseases, of the U.S. National Institutes of Health. The United States Government has certain rights to this invention.

### FIELD OF THE INVENTION

This invention relates to methods and compositions for the treatment of sepsis, inflammation or infection. In particular, the invention concerns the use of molecule(s) that target SR-BI, which is also referred to as CLA-1 (SR-BI/CLA-1), to treat sepsis, bacterial and viral infections, and inflammatory diseases. SR-BI/CLA-1 ligands contributing to the pathogenesis of disease include LPS, LTA, viral envelope proteins, beta-amyloid, serum Amyloid A and/or heat shock proteins.

### BACKGROUND OF THE INVENTION

Sepsis results from bacteria (particularly gram negative bacteria) and their products entering the bloodstream and provoking an overwhelming inflammatory response. Sepsis is classically associated with endotoxemia, an acute phase reaction, and high mortality due to disseminated intravascular coagulation, multiple organ failure and shock (Burrell, R. (1994) “HUMAN RESPONSES TO BACTERIAL ENDOTOXIN,” Circ Shock 43:137-153). Bacterial infections, as well as antibiotic therapy, cause the release of bacterial cell wall components including endotoxin (lipopolysaccharide [LPS]), lipoteichoic acid and peptidogycan (Cohen, J. et al. (1985) “ANTIBIOTIC-INDUCED ENDOTOXIN RELEASE,” Lancet 2, 1069-1070; Morrison, D. C. et al. (1987) “ENDOTOXINS AND DISEASE MECHANISMS,” Annu. Rev. Med. 38:417-432; Periti, P. et al. (1999) “NEW CRITERIA FOR SELECTING THE PROPER ANTIMICROBIAL CHEMOTHERAPY FOR SEVERE SEPSIS AND SEPTIC SHOCK,” Int. J. Antimicrob. Agents 12: 97-105) for review.

Lipopolysaccharides (LPS) are major integral components of the outer membrane of gram-negative bacteria. When released from bacteria, LPS elicit in higher organisms a broad spectrum of biological activities, especially activation of immune and inflammatory cells, including macrophages, monocytes, and endothelial cells (Guha, M. et al. (2001) “LPS INDUCTION OF GENE EXPRESSION IN HUMAN MONOCYTES,” Cell. Signal. 13:85-94; Rietschel, E. T. et al. (1992) “BACTERIAL ENDOTOXINS,” Sci. Am. 267:54-61). Systemic LPS-related activation of macrophages leads to overproduction of inflammatory mediators, such as leukocyte adhesion molecules, soluble cytokines and chemokines. LPS-activated phagocytes secrete TNF-a and IL-1β, which contributes to micro-capillary damage, plasma leakage into tissue, hypotension and organ failure, the major manifestations of septic shock (for review, see Ulevitch, R J. (2000) “MOLECULAR MECHANISMS OF INNATE IMMUNITY,” Immunol. Res. 21:49-54 and Aderem, A. et al. (2000) “TOLL-LIKE RECEPTORS IN THE INDUCTION OF THE INNATE IMMUNE RESPONSE,” Nature 406:782-787). The vascular endothelial cells, like macrophages, play a central role in a host's defense against bacterial infection and are a major cellular target for LPS action. LPS has multiple effects on macrophages, including the induction of secreted inflammatory mediators such as leukocyte adhesion molecules, soluble cytokines, and chemokines (Lynn, W. A. (1995) “ADJUNCTIVE THERAPY FOR SEPTIC SHOCK: A REVIEW OF EXPERIMENTAL APPROACHES,” Clin. Infect. Dis. 20:143-158, Rietschel, E. T. et al. (1992) “BACTERIAL ENDOTOXINS,” Sci. Am. 267:54-61, Williams, K. J. et al. (1998) “THE RESPONSE-TO-RETENTION HYPOTHESIS OF A THEROGENESIS REINFORCED,” Curr. Opin. Lipidol. 9:471-474).

The endotoxic activity of LPS, as well as its cellular uptake and metabolism, appear to be mediated by an interaction with specific cell surface receptor(s). Activation of LPS-competent cells is initiated by LPS-binding protein, which transfers LPS from the bacterial wall to membrane associated CD14. LPS-CD14 complexes signal via Toll-like receptor 4 to activate NF-KB, as well as the c-Jun N-terminal kinase, and p38 mitogen-activated protein kinases (Han, J. et al. (1997) “ACTIVATION OF THE TRANSCRIPTION FACTOR MEF2C BY THE MAP KINASE P38 IN INFLAMMATION,” Nature 386:296-299; Ulevitch, R J. et al. (1995) “RECEPTOR-DEPENDENT MECHANISMS OF CELL STIMULATION BY BACTERIAL ENDOTOXIN,” Annu. Rev. Immunol. 13:437-457; Sweet, M. J. et al. (1996) “ENDOTOXIN SIGNAL TRANSDUCTION IN MACROPHAGES,” J. Leukoc. Biol. 60:8-26).

NF-KB is a central mediator of gene expression induced by proinflammatory and proatherogenic stimuli, including inflammatory cytokines, oxidative stress, LPS, and bacterial products (Muller, J. M. et al. (1993) “NUCLEAR FACTOR KAPPA B, A MEDIATOR OF LIPOPOLYSACCHARIDE EFFECTS,” Immunobiology 187:233-256). Toll-like and interleukin receptor families deliver signals from a wide spectrum of ligands (Yang, R. B. et al. (1998) “TOLL-LIKE RECEPTOR-2 MEDIATES LIPOPOLYSACCHARIDE-INDUCED CELLULAR SIGNALING,” Nature 395:284) through downstream signaling to activate NF-KB translocation, inducing the coordinated expression of specific genes (Muller, J. M. et al. (1993) “NUCLEAR FACTOR KAPPA B, A MEDIATOR OF LIPOPOLYSACCHARIDE EFFECTS,” Immunobiology 187:233-256; Sweet, M. J. et al. (1996) “ENDOTOXIN SIGNAL TRANSDUCTION IN MACROPHAGES,” J. Leukoc. Biol. 60:8-26). The NF-KB-responsive genes (Muller, J. M. et al. (1993) “NUCLEAR FACTOR KAPPA B, A MEDIATOR OF LIPOPOLYSACCHARIDE EFFECTS,” Immunobiology 187:233-256) encoding leukocyte adhesion molecules (such as vascular cell adhesion molecular-1 and intracellular adhesion molecule-1), chemotactic factors (such as monocyte chemoattractant protein-1), and growth factors (such as macrophage colony-stimulating factor) have been demonstrated to be involved in the early development of atherosclerotic lesions (Glass, C. K. et al. (2001) “ATHEROSCLEROSIS: THE ROAD AHEAD,” Cell 104:503-516; Iiyama, K. et al. (1999) “PATTERNS OF VASCULAR CELL ADHESION MOLECULE-1 AND INTERCELLULAR ADHESION MOLECULE-1 EXPRESSION IN RABBIT AND MOUSE ATHEROSCLEROTIC LESIONS AND AT SITES PREDISPOSED To LESION FORMATION,” Circ. Res. 85:199-207; Matsushita, H. et al. (2000) “HYPOXIA-INDUCED ENDOTHELIALAPOPTOSIS THROUGH NUCLEAR FACTOR-KAPPAB (NF-KAPPAB)-MEDIATED BCL-2 SUPPRESSION: IN VIVO EVIDENCE OF THE IMPORTANCE OF NF-KAPPAB IN ENDOTHELIAL CELL REGULATION,” Circ. Res. 86:974-981; Ross, R. (1999) “ATHEROSCLEROSIS-AN INFLAMMATORY DISEASE,” N. Engl. J. Med. 340:115-126). The activation of NF-KB induces expression of genes encoding for TNF-α, IL-1β, IL-6, IL-8, leukocyte adhesion molecules (such as vascular cell adhesion molecular-1 [VCAM-1] and intracellular adhesion molecule-1 [ICAM-1]), and chemotactic factors (such as monocyte chemoattractant protein-1 [MCP-1]) that are believed to be involved with the development and progression of septic shock. However, these observations have provided little insight into an understanding of the abnormal cholesterol metabolism in lesion sites.

A large part of the host defense to septic shock involves the neutralization of LPS by its binding to high-density lipoproteins (HDL), which ultimately results in the clearance of LPS by the liver (Freudenberg, M. A. et al. (1980) “INTERACTION OF LIPOPOLYSACCHARIDES WITH PLASMA HIGH-DENSITY LIPOPROTEIN IN RATS,” Infect. Immun. 28:373-380; Parker, T. S. et al. (1995) “RECONSTITUTED HIGH-DENSITY LIPOPROTEIN NEUTRALIZES GRAM-NEGATIVE BACTERIAL LIPOPOLYSACCHARIDES IN HUMAN WHOLE BLOOD,” Infect. Immun. 63, 253-258). When incubated with human plasma, approximately 90% of LPS is associated with lipoproteins, the majority (60%) of which is associated with HDL (de Haas, C. J. et al. (2000) “ANALYSIS OF LIPOPOLYSACCHARIDE (LPS)-BINDING CHARACTERISTICS OF SERUM COMPONENTS USING GEL FILTRATION OF FITC-LABELED LPS,” J. Immunol. Methods 242:79-89; Levels, J. H. et al. (2001) “DISTRIBUTION AND KINETICS OF LIPOPROTEIN-BOUND ENDOTOXIN,” Infect. Immun. 69, 2821-2828). A smaller portion of LPS is associated with other plasma proteins including human serum albumin (HSA), LPS-binding protein and soluble CD14 (de Haas, C. J. et al. (2000) “ANALYSIS OF LIPOPOLYSACCHARIDE (LPS)-BINDING CHARACTERISTICS OF SERUM COMPONENTS USING GEL FILTRATION OF FITC-LABELED LPS,” J. Immunol. Methods 242:79-89). This non-lipoprotein associated LPS is highly inflammatory, and it appears to represent the major form of LPS contributing to sepsis. Infusion of HDL has been shown to significantly reduce the endotoxin-induced release of TNF, IL-6 and IL-8 in a murine model of endotoxemia (Levine, D. M. et al. (1993) “IN VIVO PROTECTION AGAINST ENDOTOXIN BY PLASMA HIGH DENSITY LIPOPROTEIN,” Proc. Natl. Acad. Sci. U.S.A 90:12040-12044; Pajkrt, D. et al. (1996) “ANTIINFLAMMATORY EFFECTS OF RECONSTITUTED HIGH-DENSITY LIPOPROTEIN DURING HUMAN ENDOTOXEMIA,” J. Exp. Med. 184:1601-1608) and has been proposed as a potential therapy for sepsis.

In addition to the role of LPS in provoking gram negative sepsis (Lynn, W. A. (1995) “ADJUNCTIVE THERAPY FOR SEPTIC SHOCK: A REVIEW OF EXPERIMENTAL APPROACHES,” Clin. Infect. Dis. 20:143-158), recent studies demonstrate that LPS induces immune, proinflammatory, and other as-yet-undetermined mechanisms that may be important in triggering atherogenesis (Lopes-Virella, M. F. (1993) “INTERACTIONS BETWEEN BACTERIAL LIPOPOLYSACCHARIDES AND SERUM LIPOPROTEINS AND THEIR POSSIBLE ROLE IN CORONARY HEART DISEASE,” Eur. Heart J. 14:118-124, Saikku, P. et al. (1988) “SEROLOGICAL EVIDENCE OF AN ASSOCIATION OF A NOVEL CHLAMYDIA, TW AR, WITH CHRONIC CORONARY HEART DISEASE AND ACUTE MYOCARDIAL INFARCTION,” Lancet ii:983-986). Both clinical and experimental data support considering atherosclerosis as a chronic inflammatory disorder associated with endothelial and cholesterol balance dysfunction (Danesh, J. et al. (1997) “CHRONIC INFECTIONS AND CORONARY HEART DISEASE: IS THERE A LINK”? Lancet 350:430-436; Gaydos, C. A. et al. (1996) “REPLICATION OF CHLAMYDIA PNEUMONIAE IN VITRO IN HUMAN MACROPHAGES, ENDOTHELIAL CELLS, AND AORTIC ARTERY SMOOTH MUSCLE CELLS,” Infect. Immun. 64:1614-1620). A number of inflammatory diseases, including lupus erythermatosis, arthritis, Crohn's disease, and others, have been reported to be accompanied by the premature development of atherosclerosis (Lenardo, M. J. et al. (1989) “NF-KB: A PLEIOTROPIC MEDIATOR OF INDUCIBLE AND TISSUE-SPECIFIC GENE CONTROL,” Cell 58:227-229; Liao, W. (1999) “Endotoxin: POSSIBLE ROLES IN INITIATION AND DEVELOPMENT OF ATHEROSCLEROSIS,” J. Lab. Clin. Med. 128:452-460; Petri, M. et al. (1992) “RISK FACTORS FOR CORONARY ARTERY DISEASE IN PATIENTS WITH SYSTEMIC LUPUS ERYTHEMATOSUS,” Amer. J. Med. 93:513-519).

Bacterial LPS has been demonstrated to exist in high molecular weight (up to 1000 kDa) aggregates (cell wall debris) and in a monomerized state when it forms complexes with HSA, CD14, LBP, low-density lipoproteins (LDL) or HDL. Aggregated LPS has been demonstrated to be rapidly taken up by the liver, lung and spleen, organs with large reticulo-endothelial cell populations, which abundantly express scavenger receptor class A (van Oosten, M. et al. (1998) “NEW SCAVENGER RECEPTOR-LIKE RECEPTORS FOR THE BINDING OF LIPOPOLYSACCHARIDE TO LIVER ENDOTHELIAL AND KUPFFER CELLS,” Infect. Immun 66:5107-5112; van Oosten, M. et al. (2001) “SCAVENGER RECEPTOR-LIKE RECEPTORS FOR THE BINDING OF LIPOPOLYSACCHARIDE AND LIPOTEICHOIC ACID To LIVER ENDOTHELIAL AND KUPFFER CELLS,” J. Endotoxin Res. 7:381-384). Upon intravenous injection of iodinated LPS preparations which contain both partially monomerized LPS and aggregated LPS, the uptake of aggregated LPS by the reticulo-endothelial system through scavenger receptor class A masks the participation of other receptors involved with the uptake of monomerized LPS in vivo. It has been reported that infusion of iodinated LPS monomerized by association with HDL results in an altered tissue uptake in mice (Mathison, J. C. et al. (1985) “UPTAKE AND SUBCELLULAR LOCALIZATION OF BACTERIAL LIPOPOLYSACCHARIDE IN THE ADRENAL GLAND,” Amer. J. Pathol. 120:79-86). Of significance, the association with steroid-producing tissues, such as adrenal gland and ovary was increased. These observations raise the possibility that LPS tissue targeting may also involve an HDL-receptor, such as the scavenger receptor type B class I (SR-BI/CLA-1), which is highly expressed in steroid producing tissues and the liver (for review (Trigatti, B. L. et al. (2000) “CELLULAR AND PHYSIOLOGICAL ROLES OF SR-BI, A LIPOPROTEIN RECEPTOR WHICH MEDIATES SELECTIVE LIPID UPTAKE,” Biochim. Biophys. Acta 1529:276-286)).

Lypopolysacharide represents only one example of novel pathological SR-BI/CLA-1 ligands. Several other ligands have been recently found to involve in viral infection, inflammation and inflammation-related diseases such systemic amyloidosis, Alzheimer's disease as well as HCV, include HCV E2 glycoprotein, serum amyloid A and beta-Amyloid. The binding of such ligands to CLA-1 induces direct proinflammatory reactions. With serum amyloidA there is also an association with partial amyloid degradation into potentially pro-amyloidogenic peptides which may facilitate amyloid deposition. Binding of HCV E2 glycoprotein to CLA-1 has been suggested to promote viral uptake and possibly viral fusion associated with HCV infection.

Unfortunately, however, despite all such advances, a need still remains for compositions and methods that can be used to provide a treatment for sepsis and inflammatory diseases and inflammatory conditions. The present invention is directed to this and other goals.

### BRIEF DESCRIPTION OF THE FIGURES

FIGS. 1A, 1B, 1C and 1D show the effect of LPS on the time course of SR-BI/CLA-1 (FIG. 1A), ABCA1 (FIG. 1B), and IL-β (FIG. 1C) mRNA and SR-BI/CLA-1 protein (FIG. 1D) expression. FIGS. 1A, 1B and 1C: Total mRNA is isolated from RAW cells after 0, 2, 4, 6, and 24 h of incubation with LPS (1 μg/ml) in serum-free medium. Levels of mRNA expression for the indicated genes are tested by RT-PCR analyses. Corresponding samples were analyzed for GAPDH mRNA as controls. Levels of mRNA were quantitated by scanning densitometry and corrected relative to the levels of housekeeping gene mRNA. Data are presented as the ratio of integral optical density of the indicated gene to that of the GADPH gene multiplied by 100. FIG. 1D: Cultured cells were treated with LPS for the indicated periods of time, and SR-BI protein expression was estimated by Western blot analysis. Simultaneously, β-actin levels in the corresponding samples were determined to confirm equal protein loading. Nitrocellulose membranes were scanned, and the integral optical density (IOD) values of the protein bands were estimated with the GelPro computer program. Data are expressed as the ratio of the SR-BI band integral optical density to the corresponding β-actin band integral optical density. Error bars indicate standard deviations.

FIGS. 2A, 2B, 2C and 2D show the dose-dependent effect of LPS on the expression of ABCA1 (FIG. 2A), SR-BI/CLA-1 (FIG. 2B), and IL-1β (FIG. 2C) genes and SR-BI/CLA-1 protein (FIG. 2D). Cultured cells are exposed to the increasing concentrations (0, 0.2, 2, 20, and 200 ng/ml) of LPS for 24 h. Levels of mRNA expression for the indicated genes were tested by RT-PCR analyses, and SR-BI protein expression was estimated by Western blot analysis. For further steps, see the FIG. 1 legend. The results shown represent one of two experiments that yielded similar results. IOD, integrated optical density. Error bars indicate standard deviations.

FIGS. 3A and 3B show down regulation of specific HDL binding and HDL-mediated cholesterol efflux by LPS. FIG. 3A shows the effect of LPS on 125I-HDL specific binding in RAW cells. The cells are incubated with 1 μg of LPS per ml for 24 h. Following three PBS washes, the specific binding of ¹²⁵I-HDL (5 μg/ml) was determined at 4° C. as the difference between the total and nonspecific binding (in the absence or presence of a 50-fold excess of unlabeled HDL). FIG. 3B shows the dose-dependent response of HDL-mediated [³H]cholesterol efflux to LPS stimulation. RAW cells preloaded with cholesterol are labeled with 1 μCi of [1,2-³H]cholesterol (50 Ci/mmol) per ml. Before the cholesterol efflux determination, the cells are pretreated with the increasing concentrations (0 to 1,000 ng/ml) of LPS for 24 h. After HDL (100 μg/ml) as the cholesterol acceptor was added, the [³H]cholesterol efflux assay was performed after an additional 24 h. Cholesterol efflux was calculated as the amount of radioactivity present in the medium divided by the total radioactivity (medium plus cell) in each well. The data shown represent one of two independent experiments that yielded similar results. *, P<0.05; **, P<0.01 (compared to untreated control samples). Error bars indicate standard deviations

FIGS. 4A, 4B, 4C and 4D show comparison of Re595, 0011:B4, DPLA, and MPLA potency to modulate LPS-sensitive gene expression and to suppress SR-BI/CLA-1 protein expression. RAW cells were exposed to different LPS preparations (10 ng/ml) for 24 h in serum-free medium. (FIGS. 4A, 4B and 4C) Total mRNA was isolated and treated as described in the FIG. 1 legend. The levels of mRNA expression for the indicated genes were tested by RT-PCR analyses. FIG. 4D shows the level of SR-BI/CLA-1 protein expression was determined by Western blot analysis. The results represent one of two separate experiments that yielded similar results. *, P<0.05; **, P<0.01 (compared to control untreated samples). IOD, integrated optical density. Error bars indicate standard deviations.

FIG. 5 shows the effect of protease inhibitor TPCK and its structural analogue TLCK on LPS-induced changes in IL-1[3, SR-BI/CLA-1, and ABCAI mRNA expression. The cells were incubated with TPCK or TLCK (negative control) alone for 2 h prior to LPS addition and then for 22 h in the presence of LPS (10 ng/ml). After the incubation, total mRNA was isolated and the samples were analyzed by RT-PCR. The results shown represent one of two separate experiments that yielded similar results.

FIGS. 6A and 6B show the results of LPS competition for HDL-binding sites in RAW cells. FIG. 6A: RAW cells are incubated with 1 μg/ml ¹²⁵I-HDL in the presence of various concentrations of unlabeled HDL () or LPS (◯). FIG. 6B: RAW cells were incubated with 1 μg/ml HDL and 50 μg/ml of various LPS analogs.

FIGS. 7A and 7B show the results of LPS-competition for apoA-I/apoA-II-binding sites in RAW cells. FIG. 7A: RAW cells are incubated with 1 μg/ml ¹²⁵I-HDL in the presence of various concentrations of unlabeled apoA-I () or LPS (◯). FIG. 7B: RAW cells are incubated with 1 μg/ml ¹²⁵I-HDL in the presence of various concentrations of unlabeled apoA-II () or LPS (◯).

FIGS. 8A and 8B show the effect of CLA-1 overexpression on HDL-binding and HDL-cholesterol ester uptake in HeLa cells. FIG. 8A: Specific HDL-binding in mock transfected and CLA-1 stably transfected HeLa cells. FIG. 8A: HDL-Cholesteryl oleoyl uptake from HDL in mock transfected and CLA-1 stably transfected HeLa cells.

FIGS. 9A, 9B and 9C show the effect of CLA-1 overexpression of LPS-binding in HeLa cells. FIG. 9A: Dose-dependent specific ¹²⁵I-LPS (B4:0111) binding in mock transfected and CLA-1 stably transfected HeLa cells. The competition of LPS analogues with 1 μg/ml ¹²⁵I-LPS (B4:0111) in CLA-1 stably transfected (FIG. 9B) and mock transfected (FIG. 9C) HeLa cells. Figure legend for FIGS. 9B and 9C: A (Control); B (B4:0111); C(Re595); D (DPLA); E (MPLA); F (HDL); G (ApoA-I) and H (ApoA-II).

FIGS. 10A and 10B show the effect of CLA-1 overexpression on specific LPS-binding and internalization in HeLa cells. HeLa cells are incubated with 1 μg/ml ¹²⁵I-LPS (B4:0111) in the presence or absence of 100× excess of unlabeled LPS. FIG. 10A: Trypsin-sensitive specific ¹²⁵I-LPS-uptake in mock transfected (◯) and CLA-1 stably transfected () HeLa cells. FIG. 10B: Cell associated, trypsin-resistant specific ¹²⁵I-LPS (B4:0111) uptake in mock transfected (◯) and CLA-1 stably transfected () HeLa cells.

FIGS. 11A, 11B, 11C, and 11D show selective [³H]LPS uptake and metabolism of LPS-labeled HDL in HeLa cells. To measure selective LPS uptake, cells are cultured in 12-well cluster plates and incubated at 37° C. in 0.5 ml of medium containing 10 μg of ¹²⁵I-HDL, LPS-¹²⁵I-HDL complex (FIG. 11A), [¹⁴C]CE- or ³H-LPS-labeled HDL (FIG. 11B). After 1 h of incubation, the cells are washed, lysed in NaOH, and the radioactivity in the cellular lysate is counted. To determine the amount of apolipoprotein degradation, the cells are pulsed with 10 μg of ¹²⁵I-HDL (FIG. 11C) or LPS-¹²⁵I-HDL complex (FIG. 11D) and chased into fresh media for 2 h. The amount of internalized, secreted, and degraded 125I-apolipoproteins was determined as described under “Materials and Methods” in Example 2.

FIGS. 12A, 12B and 12C show that amphipathic double helix containing peptides compete for LPS-binding sites in CLA-1 overexpressing and mock transfected HeLa cells. LPS-binding is measured in CLA-1 overexpressing and mock transfected HeLa cells after incubation with 1 μg/ml ¹²⁵I-LPS in the presence of various concentrations of unlabeled LPS (FIG. 12A), L-37PA (FIG. 12B) or D-37PA (FIG. 12C).

FIG. 13 concerns amphipathic peptides containing a double helix block Bodipy LPS uptake in HeLa cells. Bodipy-LPS uptake is measured by a fluorescent spectrophotometer in the presence of various concentrations of L-37PA, D-37PA, L2D-37PA and 18PA in HeLa cells.

FIGS. 14A and 14B concern amphipathic peptides containing a double helix block cytokine production in THP-1 cells. THP-1 cells are incubated with 10 ng/ml 0111:B4 LPS in the presence or absence of various concentrations of L-37PA, D-37PA, L2D-37PA or 18PA for 24 hs. After the media is harvested, IL-8 (FIG. 14A) and IL-6 (FIG. 14B) is measured by ELISA. Legend: L37PA (□); D37PA (◯); L2D37PA (Δ); and L18PA (∇).

FIGS. 15A, 15B, 15C and 15D concern amphipathic peptides containing a double helix block cytokine production induced by lipoteichoic acid and bacterial heat shock protein 60 (Gro-EL) in THP-1 cells. THP-1 cells are incubated with no additions, 10 ng/ml LPS, 1 μg/ml LTA or 50 ng/ml untreated and heat denatured (20 min, 100° C.) E. coli Gro-EL in the presence or absence of 10 μg/ml L-37PA, D-37PA, L1D-37PA, L2D-37PA or L3D-37PA for 24 h in DMEM containing 1% FCS. The amounts of IL-8 (FIGS. 15A and 15C) as well as IL-6 (FIGS. 15B and 15D) are measured by ELISA in conditioned media after a 24-h incubation.

FIGS. 16A and 16B show FACS analyses of Alexa 568-SAA and Alexa 568-HDL uptake in Hela cells. HeLa cells are incubated with increasing concentrations of Alexa 488-SAA or Alexa 488-HDL for 2-hours. After incubation, cells are washed with PBS, released from the culture plate with an EDTA containing solution, fixed with 4% paraformaldehyde and analyzed by FACS. Data represent Alexa 488-SAA (FIG. 16A) and Alexa 488-HDL (FIG. 16B) uptake. Data represents the mean (SD) of three independent experiments.

FIGS. 17A and 17B show competition of CLA-1 ligands for SAA-uptake in HeLa cells CLA-1 overexpressing (FIG. 17A) and mock-transfected cells (FIG. 17B) are incubated with 1 μg/ml Alexa 488-SAA in the presence or absence of increasing concentrations of competitors. Cells are treated as described in the Methods section and analyzed by FACS. Data represents one of three represented experiments.

### SUMMARY OF THE INVENTION

This invention relates to methods and compositions for the treatment of sepsis, inflammation or infection. In particular, the invention concerns the use of molecules that target SR-BI/CLA-1 to treat sepsis, bacterial and viral infections, and inflammatory diseases. SR-BI/CLA-1 ligands contributing to the pathogenesis of disease include LPS, LTA, viral envelope proteins, beta-amyloid, serum Amyloid A and/or heat shock proteins.

In detail, the invention concerns a method for the treatment of sepsis, inflammation or infection comprising providing to a recipient (including humans, cattle, sheep, pigs, dogs, cats, etc.) a physiologically effective amount of a pharmaceutical composition comprising a molecule that targets SR-BI/CLA-1. The invention particularly concerns the embodiment of such method wherein the pharmaceutical composition binds to SR-BI/CLA-1 with a Kd lower than 10⁻⁷ M and competes against pathogenic molecules, or affects the function or expression level of SR-BI/CLA-1. The pharmaceutical composition may function as an SR-BI/CLA-1 antagonist and/or as an agent which disrupt plasma membrane microorganization preventing normal SR-BI/CLA-1 function.

The invention particularly concerns the embodiments of such methods wherein the method provides a treatment for sepsis arising from endotoxemia that results from an acute phase reaction to the presence of bacteria (particularly gram negative bacteria) and their products in the bloodstream of a mammal. The invention additionally concerns the embodiments of such methods wherein the method provides a treatment for inflammation that is caused by a reaction of the specific defense system or the non-specific defense system. The reaction may be also induced by other pathological molecules which specially bind to SR-BI/CLA-1/CLA-1 such as serum Amyloid A, beta-amyloid and other agents. The invention also concerns the embodiments of such methods wherein the method provides a treatment for infection, especially infection caused by bacteria (especially Enteropathogenic Escherichia coli; Enterohamorrhagic Escherichia coli; Chlamydia etc.) or viruses (especially, Human Immunodeficiency Virus (HIV); Human Hepatitis C Virus (HCV); Ebola virus; Marburg virus, etc.).

The invention concerns the embodiments of all such methods wherein the molecule is a peptide or is a peptide composition having a peptide portion, and especially wherein the peptide or peptide composition effects LPS-uptake or LPS-stimulated cytokine production and/or targets SR-BI/CLA-1 by binding with a Kd lower than 10⁻⁷ M. The invention concerns the embodiments of all such methods wherein the peptide (or the peptide component of a peptide composition) is composed solely of L- or of D-amino acid residues. The invention concerns the embodiments of all such methods wherein the peptide binds to SR-BI/CLA-1 with Kd lower than 10⁻⁷ M.

The invention further concerns the embodiments of all such methods wherein the molecule of the pharmaceutical composition is selected from the group consisting of a cholesterol absorption inhibitor, a viral fusion inhibitor, a negatively charged lipid that binds to CLA-1 with a Kd lower than 10⁻⁷ M; an anti-SR-BI/CLA-1 antibody, of fragment thereof that binds SR-BI/CLA-1, and a chemical substance that binds to SR-BI/CLA-1 with a Kd lower than 10⁻⁷ M.

The invention additionally provides a pharmaceutical composition for the treatment of sepsis, inflammation or infection comprising
(A) a physiologically effective amount of a molecule that targets SR-BI/CLA-1; and
(B) an auxiliary agent, excipient, or uptake facilitating agent.

The invention includes the embodiments of such pharmaceutical compositions wherein the molecule that targets SR-BI/CLA-1 does so by binding to SR-BI/CLA-1. The invention particularly concerns the embodiments of such pharmaceutical compositions wherein the physiologically effective amount of the pharmaceutical composition is effective for providing a treatment for sepsis arising from endotoxemia that results from an acute phase reaction to the presence of bacteria (particularly gram negative bacteria) and their products in the bloodstream of a mammal. The invention additionally concerns the embodiments of such pharmaceutical composition wherein the physiologically effective amount of the pharmaceutical composition is effective for providing a treatment for inflammation that is caused by a reaction of the specific defense system or the non-specific defense system. The invention also concerns the embodiments of such methods wherein the physiologically effective amount of the pharmaceutical composition is effective for providing a treatment for infection, especially infection caused by bacteria (especially Enteropathogenic Escherichia coli; Enterohamorrhagic Escherichia coli; Chlamydia etc.) or viruses (especially, Human Immunodeficiency Virus (HIV); Human Hepatitis C Virus (HCV); Ebola virus; Marburg virus, etc.).

The invention concerns the embodiments of all such pharmaceutical compositions wherein the molecule of said pharmaceutical composition is a peptide or is a peptide composition having a peptide portion, and especially wherein such peptide or peptide composition effects LPS-uptake or LPS-stimulated cytokine production and/or targets SR-BI by binding with Kd less than 10⁻⁷ M. The invention concerns the embodiments of all such pharmaceutical compositions wherein the molecule is a peptide or peptide composition, and wherein such peptide (or the peptide component of such peptide composition) is composed solely of L- or of D-amino acid residues.

The invention further concerns the embodiments of all such pharmaceutical compositions wherein the molecule of the pharmaceutical composition is selected from the group consisting of a cholesterol absorption inhibitor, a viral fusion inhibitor, a negatively charged lipid that binds to CLA-1 with a Kd lower than 10⁻⁷ M; an anti-SR-BI/CLA-1 antibody, of fragment thereof that binds SR-BI, and a chemical substance that binds to SR-BI/CLA-1 with a Kd lower than 10⁻⁷ M.

### DESCRIPTION OF THE PREFERRED EMBODIMENTS OF THE INVENTION

This invention relates to methods and compositions for the treatment of sepsis, inflammation or infection. In particular, the invention concerns the use of molecules that target SR-BI/CLA-1 to treat sepsis, bacterial and viral infections, and inflammatory diseases. SR-BI/CLA-1 ligands contributing to the pathogenesis of disease include LPS, LTA, viral envelope proteins, beta-amyloid, serum Amyloid A and/or heat shock proteins.

As used herein, the term “treatment” is intended to refer to the administration of a “pharmacologically acceptable” amount of a physiologically significant agent for either a “prophylactic” or “therapeutic” purpose. An agent is physiologically significant if its presence results in a detectable change in the physiology of a recipient patient. The compositions of the present invention are said to be administered in a “therapeutically effective amount” if the amount administered is sufficient to provide a therapy for an actual infection. When provided for a therapeutic purpose, the compound is preferably provided at (or shortly after) the onset of a symptom of actual sepsis or inflammation. The therapeutic administration of the compound serves to attenuate an actual occurrence of sepsis or inflammation. The compositions of the present invention are said to be administered in a “prophylactically effective amount” if the amount administered is sufficient to provide a therapy for a potential infection. When provided for a prophylactic purpose, the compound is preferably provided in advance of any symptom of sepsis or inflammation. The prophylactic administration of the compound serves to prevent or attenuate subsequent sepsis or inflammation.

The term “sepsis” is intended to refer to the endotoxemia, the acute phase reaction to the presence of bacteria (particularly gram negative bacteria) and their products in the bloodstream of a mammal (including humans, cattle, sheep, pigs, dogs, cats, etc.).

The term “inflammation” as used herein, is meant to include both the reactions of the specific defense system, and the reactions of the non-specific defense system. As used herein, the term “specific defense system” is intended to refer to that component of the immune system that reacts to the presence of specific antigens. Inflammation is said to result from a response of the specific defense system if the inflammation is caused by, mediated by, or associated with a reaction of the specific defense system. Examples of inflammation resulting from a response of the specific defense system include the response to antigens such as rubella virus, autoimmune diseases such as lupus erythematosus, rheumatoid arthritis, Reynaud's syndrome, multiple sclerosis etc., delayed type hypersensitivity response mediated by T-cells, etc. Chronic inflammatory diseases and the rejection of transplanted tissue and organs are further examples of inflammatory reactions of the specific defense system.

The term “infection” is intended to refer to microbial infection generally, and in particular to encompass infection caused by bacteria (especially Enteropathogenic Escherichia coli; Enterohamorrhagic Escherichia coli; Chlamydia etc.) or viruses (especially, Human Immunodeficiency Virus (HIV); Human Hepatitis C Virus (HCV); Ebola virus; Marburg virus, etc.).

As used herein, a reaction of the “non-specific defense system” is intended to refer to a reaction mediated by leukocytes incapable of immunological memory. Such cells include granulocytes and macrophages. As used herein, inflammation is said to result from a response of the non-specific defense system, if the inflammation is caused by, mediated by, or associated with a reaction of the non-specific defense system. Examples of inflammation which result, at least in part, from a reaction of the non-specific defense system include inflammation associated with conditions such as: adult respiratory distress syndrome (ARDS) or multiple organ injury syndromes secondary to septicemia or trauma; reperfusion injury of myocardial or other tissues; acute glomerulonephritis; reactive arthritis; dermatoses with acute inflammatory components; acute purulent meningitis or other central nervous system inflammatory disorders; thermal injury; hemodialysis; leukophoresis; ulcerative colitis; Crohn's disease; necrotizing enterocolitis; granulocyte transfusion associated syndromes; and cytokine-induced toxicity.

As used herein, a molecule is said to “target” SR-BI/CLA-1 (or to be an “SR-BI/CLA-1 targeting molecule”) if it is capable of binding to SR-BI/CLA-1 or affecting its activity. If binding is the mechanism, it should be sufficient to displace pathogenic molecules from binding to SR-BI/CLA-1CLA-1 or to interfere with the binding of such pathogenic molecules to SR-BI/CLA-1/CLA-1. Most preferably, molecules target SR-BI/CLA-1 by binding to SR-BI/CLA-1 with a Kd lower than 10⁻⁷ M. Such binding may be mediated by any of a variety of mechanisms. Human scavenger receptor class B type I, referred to as CLA-1, is a high density lipoprotein (HDL) receptor whose primary function is HDL binding and selective HDL cholesterol ester uptake. As used herein, a molecule is said to have a “motif targeting SR-BI/CLA-1” if it contains both hydrophobic and hydrophilic regions and binds to human scavenger receptor class B type I (CLA-1) under physiological conditions with Kd lower than 10⁻⁷ M. The major CLA-1 recognition motif is an amphipathic helical sequence, which is a common feature of exchangeable apolipoproteins as well as several proinflammatory proteins including heat shock proteins and the hypervariable region 1 of hepatitis C virus.

It has been found that human scavenger receptor class B type I, CLA-1, mediates LPS-binding and internalization (Vishnyakova, T. G. et al. (2003) “BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780). The present invention extends the finding that the major recognition motif in SR-BI ligands is the anionic amphipathic α-helix, to find that molecules that target SR-BI/CLA-1 include amphipathic α-helical containing peptides, and that such targeting has an effect on LPS-uptake and LPS stimulated cytokine production.

The ability of molecules that target SR-BI/CLA-1 to affect the capacity of SR-BI/CLA-1 to bind pathogenic materials is illustrated below using model peptides that possess amphipathic helices. L-37PA peptide contains two class A amphipathic helices, and efficiently competes against iodinated LPS in both mock transfected and CLA-1 overexpressing HeLa cells. Alexa-L-37PA and monomeric Bodipy-LPS co-localizes at the cell surface and intracellular perinuclear compartment. Both ligands are predominantly transported to the Golgi complex, co-localizing with BSA-ceramide, a Golgi marker. A 100-fold excess of L-37PA nearly eliminated Bodipy-LPS cellular uptake. L-37PA as well as the D-amino acid D-37PA peptide described herein are similarly effective in blocking LPS, gram-positive bacterial wall component lipoteichoic acid (LTA) and bacterial heat shock protein Gro-EL-stimulated cytokine secretion in THP-1 cells. When utilizing the same culture media used for the cytokine stimulation study, neither L-37PA nor D-37PA affected LPS's endotoxin activity as determined by the Limulus amebocyte lysate (LAL) assay. This unaffected endotoxin activity indicates that amphipathic helical peptides can block LPS uptake and cytokine stimulation independently of LPS-neutralization. These results demonstrate that the amphipathic helical motif of exchangeable apolipoproteins may represent a general host defense mechanism against inflammatory reactions and indicate that agents targeting CLA-1 represent a new class of therapeutics for infections and inflammation.

Serum Amyloid A (SAA) is an acute phase reactant and proinflammatory molecule which is also characterized by the presence of an amphipathic helical motif. Flow cytometry experiments demonstrated more than a 5-fold increase of Alexa-488 SAA uptake in CLA-1 stably transfected HeLa cells when compared with mock transfected HeLa cells. SAA uptake was dose-dependent and plateaued at a concentration of 2.5-5 μg/ml. ApoA-I, the major HDL apolipoprotein, unlabeled SAA and the amphipathic helical peptide L-37PA competed for CLA-1 binding with Alexa 488-SAA. Alexa-488 SAA was rapidly internalized in CLA-1 overexpressing cells and transported predominantly to the transferrin-recycling compartment and to a lesser extent to either the lysosomal compartment or the Golgi complex. In CLA-1 overexpressing cells, lipoprotein free SAA degraded into smaller peptides with molecular masses between 6-8 kDa, which were rapidly resecreted into the culture media. SAA-association with HDL decreased SAA uptake and diminished SAA-degradation and resecretion by CLA-1. These data indicate that CLA-1 functions as an important receptor.

The interaction of high-density lipoproteins (HDL) or lipid poor apolipoproteins with specific surface receptors has been reported to activate cholesterol translocation to the cell surface (Yokoyama, S. (1998) “APOLIPOPROTEIN-MEDIATED CELLULAR CHOLESTEROL EFFLUX,” Biochim. Biophys. Acta 1392:1-15) and its diffusion onto appropriate particles against a cholesterol concentration gradient (Rothblat, G. H. (1999) “CELL CHOLESTEROL EFFLUX: INTEGRATION OF OLD AND NEW OBSERVATIONS PROVIDES NEW INSIGHTS,” J. Lipid Res. 40:781-796). Cholesterol efflux allows peripheral cells to eliminate cholesterol excess, which is transported to the liver for further secretion into the bile or conversion into bile acids (Mendez, A. J. (1997) “CHOLESTEROL EFFLUX MEDIATED BY APOLIPOPROTEINS IS AN ACTIVE CELLULAR PROCESS DISTINCT FROM EFFLUX MEDIATED BY PASSIVE DIFFUSION,” J. Lipid Res. 38:1807-1821; Oram, J. F. et al. (1996) “APOLIPOPROTEIN-MEDIATED REMOVAL OF CELLULAR CHOLESTEROL AND PHOSPHOLIPIDS,” J. Lipid Res. 37:2473-2491). The intracellular cholesterol pool, which is readily available for both esterification and transportation to the plasma membrane, may play a central role in macrophage transformation into foam cells. Consequently, mechanisms mediating cholesterol efflux are critical for maintaining cholesterol homeostasis in the macrophage (Glass, C. K. et al. (2001) “ATHEROSCLEROSIS: THE ROAD AHEAD,” Cell 104:503-516).

Earlier studies have demonstrated that LPS, as a potent NF-KB activator, is able to alter lipid metabolism in macrophages (Spinelle-Jaegle, S. et al. (2001) “INFLAMMATORY CYTOKINE PRODUCTION IN INTERFERON-Γ-PRIMED MICE, CHALLENGED WITH LIPOPOLYSACCHARIDE INHIBITION BY SK&F 86002 INTERLEUKIN-1B-CONVERTING ENZYME INHIBITOR,” Eur. Cytokine Netw 2:280). A several fold increase of triglyceride content and the cholesterol esterification rate has been reported to transform macrophages into foam-like cells (Glass, C. K. et al. (2001) “ATHEROSCLEROSIS: THE ROAD AHEAD,” Cell 104:503-516). However, the question of whether this observation involved expression of multiple HDL receptors has not been addressed. In the present study, the effect of LPS on the expression of the two primary HDL receptors, scavenger receptor BI (SR-BI/CLA-1) and ATP binding cassette A1 (ABCA1), in the mouse monocyte-macrophage RAW cell line is investigated.

The present invention extends such studies to identify the involvement of NF-KB activation in the LPS-induced decrease of SR-BI/CLA-1 and ABCA1 transporter expression (Baranova I. et al. (2002) “LIPOPOLYSACCHARIDE DOWN REGULATES BOTH SCAVENGER RECEPTOR BI AND ATP BINDING CASSETTE TRANSPORTER A1 IN RAW CELLS,” Infect. Immun. 2002 June; 70 (6):2995-3003; Vishnyakova, T. et al. (2003) “BINDING AND INTERNALIZATION OF LIPOPOLYSACCHARIDE BY CLA-1, A HUMAN HOMOLOGE OF RODENT SCAVENGER RECEPTOR B1.” (J Biol Chem. 20; 278 (25):22771-80; Epub 2003 Mar. 21); Remaley A. T. et al. (2003) “SYNTHETIC AMPHIPATHIC HELICAL PEPTIDES PROMOTE LIPID EFFLUX FROM CELLS BY AN ABCA1-DEPENDENT AND AN ABCA1-INDEPENDENT PATHWAY,” J Lipid Res. 44 (4):828-36; Epub 2003 Jan. 16); Baranova, I. et al. (2002) “LIPOPOLYSACCHARIDE DOWN REGULATES BOTH SCAVENGER RECEPTOR BI AND ATP BINDING CASSETTE TRANSPORTER A1 IN RAW CELLS,” Infect Immun. 70 (6):2995-3003).

Proinflammatory bacterial cell wall components including lipopolysaccharide (LPS), lipoteichoic acid (L T A) and peptidoglycan (PGN) have been found to be major factors determining the development, progression and outcome for a number of infectious diseases. Chaperonin 60 (cpn60), another bacterial component, and its human ortholog heat shock protein 60 (hsp60), also play an important role in inflammatory diseases such as arthritis and lupus erythematosus. Recently, the human scavenger receptor class B type I (SR-BI/CLA-1) was found to function as a receptor for LPS, bacterial cpn60 and human hsp60. SR-BI/CLA-1 is a receptor for high-density lipoproteins (HDL) as well as apolipoproteins AI and AII. Amphipathic helices in apolipoproteins are identified as the structural determinants that confer binding specificity. Peptides with an amphipathic helical motif, block cellular uptake of the LPS and proinflammatory responses induced by LPS, LTA, bacterial cpn60 and human hsp60 in vitro. Cellular uptake of viral envelope proteins is mediated by SR-BI/CLA-1 and can be blocked by amphipathic peptides. These observations indicate that agents with an amphipathic motif targeting SR-BI/CLA-1 can be used to treat sepsis, bacterial and viral infections, and inflammatory diseases in which LPS, LTA, viral envelope proteins, and/or heat shock proteins contribute to pathogenesis. Utilizing the principle of SR-BI/CLA-1 targeting, this recognition permits one to employ the principles of the present invention to create a number of novel compounds effective against a variety of infectious and inflammatory diseases. These effective compounds can be identified by evaluating their SR-BI/CLA-1 binding activity in vitro.

The amelioration of inflammatory responses induced by bacterial cell components and human proinflammatory factors by blocking scavenger Receptor Class B, type-I creates a new class of drugs. Pathological conditions induced by bacterial infection including hemorrhagic shock, inflammatory bowel diseases, sepsis, etc. Since viruses utilize SR-BI/CLA-1 for entry into cells, amphipathic compounds including helical peptides can be used as a treatment for viral infections.

Scavenger receptor B type (SR-BI/CLA-1) is a well-characterized HDL receptor that is highly expressed in the liver and steroidogenic tissues, including the adrenal, which is often affected during endotoxemia (Munford, R. S. et al. (1981) “SITES OF TISSUE BINDING AND UPTAKE IN VIVO OF BACTERIAL LIPOPOLYSACCHARIDE-HIGH DENSITY LIPOPROTEIN COMPLEXES: STUDIES IN THE RAT AND SQUIRREL MONKEY,” J. Clin. Invest. 68:1503-1513). Its human orthologue, CD36 and LIMPII Analogous-1 (CLA-1), has also been shown as a human receptor for high density lipoprotein and apoptotic thymocytes (Murao, K. et al. (1997) “CHARACTERIZATION OF CLA-1, A HUMAN HOMOLOGUE OF RODENT SCAVENGER RECEPTOR BI, AS A RECEPTOR FOR HIGH DENSITY LIPOPROTEIN AND APOPTOTIC THYMOCYTES,” J. Biol. Chem. 272:17551-17557). Despite the fact that CLA-1 has not been studied as extensively as rodent SR-BI, the physiological role of CLA-1 is generally assumed to be similar to that of rodent SR-BI/CLA-1. SR-BI/CLA-1's primary function has been previously demonstrated to be a selective uptake of HDL free cholesterol and cholesteryl ester without the concomitant uptake of HDL apolipoproteins, which serve as ligands for SR-BI/CLA-1 (Xu, S. et al. (1997) “APOLIPOPROTEINS OF HDL CAN DIRECTLY MEDIATE BINDING TO THE SCAVENGER RECEPTOR SR-BI, AN HDL RECEPTOR THAT MEDIATES SELECTIVE LIPID UPTAKE,” J. Lipid Res. 38:1289-1298; Thuahnai, S. T. et al. (2001) “SCAVENGER RECEPTOR CLASS B, TYPE I-MEDIATED UPTAKE OF VARIOUS LIPIDS INTO CELLS. INFLUENCE OF THE NATURE OF THE DONOR PARTICLE INTERACTION WITH THE RECEPTOR,” J. Biol. Chem. 276:43801-43808). The class A amphipathic α-helices of exchangeable apolipoproteins serve as the primary recognition motif for the interaction of HDL with SR-BI/CLA-1 (Williams, D. L. et al. (2000) “ROLES OF SCAVENGER RECEPTOR BI AND APO A-I IN SELECTIVE UPTAKE OF HDL CHOLESTEROL BY ADRENAL CELLS,” Endocr. Res. 26:639-651; Schulthess, G. et al. (2000) “INTESTINAL STEROL ABSORPTION MEDIATED BY SCAVENGER RECEPTORS IS COMPETITIVELY INHIBITED BY AMPHIPATHIC PEPTIDES AND PROTEINS,” Biochemistry 39:12623-12631). However, lipid composition (especially the presence of negatively charged phospholipids) impacts HDL-binding with SR-BI/CLA-1. Moreover, phospholipid vesicles containing no apolipoproteins, only negatively charged aminophospholipids, such as phosphotidyl serines and, phospholipids containing a negative charge such as phosphotidyl ethanolamines, as well as the phospholipid probe Dil are also effective ligands for SR-BI/CLA-1 (Urban, S. et al. (2000) “SCAVENGER RECEPTOR BI TRANSFERS MAJOR LIPOPROTEIN-ASSOCIATED PHOSPHOLIPIDS INTO THE CELLS,” J. Biol. Chem. 275:33409-33415; Thuahnai, S. T. et al. (2001) “SCAVENGER RECEPTOR CLASS B, TYPE I-MEDIATED UPTAKE OF VARIOUS LIPIDS INTO CELLS. INFLUENCE OF THE NATURE OF THE DONOR PARTICLE INTERACTION WITH THE RECEPTOR,” J. Biol. Chem. 276:43801-43808). Lipid A, the most conserved portion of endotoxin, is a phosphorylated glucosamine-based phospholipid, which resembles the physico-chemical properties of phospholipids containing a negative charge, and may function as an independent ligand for SR-BI/CLA-1 in adrenal epithelial cells, macrophages and hepatocytes, the cells that highly express SR-BI/CLA-1. Additionally SR-BI/CLA-1 can be involved with the selective uptake and excretion of HDL-associated LPS in the liver, an important mechanism of LPS clearance (Read, T. E. et al. (1993) “THE PROTECTIVE EFFECT OF SERUM LIPOPROTEINS AGAINST BACTERIAL LIPOPOLYSACCHARIDE,” Eur. Heart J. 14 Suppl K, 125-129). One aspect of the present invention concerns the role of CLA-1 in LPS metabolism and demonstrate that CLA-1 mediates the binding, endocytosis and the cellular accumulation of both monomerized, lipoprotein free LPS as well as LPS associated with HDL.

Compositions of the Present Invention

As indicated above, molecules that have an amphipathic motif are capable of targeting SR-BI/CLA-1. In one embodiment of the present invention, molecules that target SR-BI/CLA-1 may be administered to a recipient prior to the commencement of sepsis, inflammation or infection, or subsequent to the onset of such conditions. In accordance with the preferred embodiments of the invention, such a molecule could be a peptide. The invention contemplates that the molecule may comprise a single peptide that targets SR-BI/CLA-1, a peptide construct having a peptide portion that targets SR-BI/CLA-1, or a composition comprising such a peptide or peptide construct but that contains more than one molecule that targets SR-BI/CLA-1. The molecules of such compositions that target SR-BI/CLA-1 molecules may be the same or different, and may be co-administered or sequentially administered.

The peptide molecules of the invention may be prepared using virtually any art-known technique for the preparation of peptides. For example, the peptides may be prepared using conventional step-wise solution or solid phase peptide syntheses, or recombinant DNA techniques. Peptides may be prepared using conventional step-wise solution or solid phase synthesis (see, e.g., Merrifield, R B. (1969) “SOLID-PHASE PEPTIDE SYNTHESIS,” Adv. Enzymol. Relat Areas Mol. Biol. 32:221-296; Fairwell, T. et al. (1987) “HUMAN PLASMA APOLIPOPROTEIN C-II: TOTAL SOLID-PHASE SYNTHESIS AND CHEMICAL AND BIOLOGICAL CHARACTERIZATION,” Proc. Natl. Acad. Sci. U.S. A 84:4796-4800; Kent, S. B. H. “CHEMICAL SYNTHESIS OF PEPTIDES AND PROTEINS,” (1988) Ann. Rev. Biochem. 57, 957-984, CHEMICAL APPROACHES TO THE SYNTHESIS OF PEPTIDES AND PROTEINS, Williams et al., Eds., 1997, CRC Press, Boca Raton Fla., and references cited therein; SOLID PHASE PEPTIDE SYNTHESIS: A PRACTICAL APPROACH, Atherton & Sheppard, Eds., 1989, IRL Press, Oxford, England, and references cited therein).

Alternatively, the peptides of the invention may be prepared by way of segment condensation, as described, for example, in Schnölzer, M. et al., “CONSTRUCTING PROTEINS BY DOVETAILING UNPROTECTED SYNTHETIC PEPTIDES: BACKBONE-ENGINEERED HIV PROTEASE,” Science. 1992 Apr. 10; 256 (5054):221-5; Schnölzer, M., “IN SITU NEUTRALIZATION IN BOC-CHEMISTRY SOLID PHASE PEPTIDE SYNTHESIS. RAPID, HIGH YIELD ASSEMBLY OF DIFFICULT SEQUENCES,” Int J Pept Protein Res. 1992 September-October; 40 (3-4): 180-193; Rose et al., “STEPWISE SOLID-PHASE SYNTHESIS OF POLYAMIDES AS LINKERS,” J. Am. Chem. Soc. 1999 Aug. 4, 121:7034-7038. Methods for preparing and using peptide inhibitors that may be adapted to the methods of the present invention are disclosed in U.S. Pat. Nos. 6,004,925 and 6,429,289.

Where molecules that target SR-BI/CLA-1 are to be administered as a pharmaceutical composition, such composition can be formulated according to known methods for preparing pharmaceutical compositions, whereby the substance to be delivered is combined with a pharmaceutically acceptable carrier vehicle. Suitable vehicles and their preparation are described, for example, in Remington's Pharmaceutical Sciences, 19^{th} Edition, A. R. Gennaro, Ed., Mack Publishing Co., Easton, Pa. (1995).

The amount of an active agent (i.e., the molecule that targets SR-BI/CLA-1) in such a composition depends upon factors including the age and weight of the subject, the delivery method and route, the type of treatment desired, and the type of peptide or peptide construct or other molecule being administered. In general, a composition of the present invention that includes peptide or peptide constructs will contain from about 1 ng to about 30 mg of such peptide or peptide construct, more preferably, from about 100 ng to about 10 mg of such peptide or peptide construct. Certain preferred compositions of the present invention may include about 1 ng of such peptide or peptide construct, about 5 ng of such peptide or peptide construct, about 10 ng of such peptide or peptide construct, about 50 ng of such peptide or peptide construct, about 100 ng of such peptide or peptide construct, about 500 ng of such peptide or peptide construct, about 1 μg of such peptide or peptide construct, about 5 μg of such peptide or peptide construct, about 10 μg of such peptide or peptide construct, about 50 μg of such peptide or peptide construct, about 100 μg of such peptide or peptide construct, about 150 μg of such peptide or peptide construct, about 200 μg of such peptide or peptide construct, about 250 μg of such peptide or peptide construct, about 300 μg of such peptide or peptide construct, about 350 μg of such peptide or peptide construct, about 400 μg of such peptide or peptide construct, about 450 μg of such peptide or peptide construct, about 500 μg of a polynucleotide, about 550 μg of such peptide or peptide construct, about 600 μg of such peptide or peptide construct, about 650 μg of such peptide or peptide construct, about 700 μg of such peptide or peptide construct, about 750 μg of such peptide or peptide construct, about 800 μg of such peptide or peptide construct, about 850 μg of a peptide, about 900 μg of such peptide or peptide construct, about 950 μg of such peptide or peptide construct, about 1 μg of such peptide or peptide construct, about 5 mg of such peptide or peptide construct, about 10 mg of such peptide or peptide construct, about 15 mg of such peptide or peptide construct, about 20 mg of such peptide or peptide construct, about 25 mg of such peptide or peptide construct, or about 30 mg of such peptide or peptide construct.

Such molecules may be formulated into any of various compositions and may be used in any of the methods disclosed herein. For aqueous compositions used in vivo, use of sterile pyrogen-free water is preferred. Such formulations will contain an effective amount of such peptide or peptide construct together with a suitable salt and/or auxiliary agent as disclosed herein, in order to prepare pharmaceutically acceptable compositions suitable for optimal administration to a vertebrate. Insoluble peptide or peptide constructs may be solubilized in a weak acid or weak base, and then diluted to the desired volume, for example, with an aqueous solution of the present invention. The pH of the solution may be adjusted as appropriate. In addition, a pharmaceutically acceptable additive can be used to provide an appropriate osmolarity.

As used herein a “salt” is a substance produced from the reaction between acids and bases which comprises a metal (cation) and a nonmetal (anion). Salt crystals may be “hydrated” i.e., contain one or more water molecules. Such hydrated salts, when dissolved in an aqueous solution at a certain molar concentration, are equivalent to the corresponding anhydrous salt dissolved in an aqueous solution at the same molar concentration. For the present invention, salts which are readily soluble in an aqueous solution are preferred.

The terms “saline” or “normal saline” as used herein refer to an aqueous solution of about 145 mM to about 155 mM sodium chloride, preferably about 154 mM sodium chloride. The terms “phosphate buffered saline” or PBS” refer to an aqueous solution of about 145 mM to about 155 mM sodium chloride, preferably about 154 sodium chloride, and about 10 mM sodium phosphate, at a pH ranging from about 6.0 to 8.0, preferably at a pH ranging from about 6.5 to about 7.5, most preferably at pH 7.2.

Such compositions of the present invention may include one or more uptake facilitating materials that facilitate delivery of peptides or peptide constructs to the interior of a cell, and/or to a desired location within a cell. Examples of the uptake facilitating materials include, but are not limited to lipids, preferably cationic lipids; inorganic materials such as calcium phosphate, and metal (e.g., gold or tungsten) particles (e.g., “powder” type delivery solutions); peptides, including cationic peptides, targeting peptides for selective delivery to certain cells or intracellular organelles such as the nucleus or nucleolus, and amphipathic peptides, i.e. helix forming or pore forming peptides; basic proteins, such as histories; asialoproteins; viral proteins (e.g., Sendai virus coat protein); pore-forming proteins; and polymers, including dendrimers, star-polymers, “homogenous” poly-amino acids (e.g., poly-lysine, poly-arginine), “heterogenous” poly-amino acids (e.g., mixtures of lysine & glycine), co-polymers, polyvinylpyrrolidinone (PVP), and polyethylene glycol (PEG). Furthermore, those auxiliary agents of the present invention which facilitate and enhance the entry of a peptide or peptide construct into vertebrate cells in vivo, may also be considered “uptake facilitating materials.”

Certain embodiments of the present invention may include lipids as a uptake facilitating material, including cationic lipids (e.g., DMRIE, DOSPA, DC-Chol, GAP-DLRIE), basic lipids (e.g., steryl amine), neutral lipids (e.g., cholesterol), anionic lipids (e.g., phosphatidyl serine), and zwitterionic lipids (e.g., DOPE, DOPC).

Examples of cationic lipids are 5-carboxyspermylglycine dioctadecylamide (DOGS) and dipalmitoyl-phophatidylethanolamine-5-carboxy-spermylamide (DPPES). Cationic cholesterol derivatives are also useful, including {3β-[N—N′,N′-dimethylamino)ethane]-carbomoyl}-cholesterol (DC-Chol). Dimethyldioctdecyl-ammonium bromide (DDAB), N-(3-aminopropyl)-N,N-(bis-(2-tetradecyloxyethyl))-N-methyl-ammonium bromide (PADEMO), N-(3-aminopropyl)-N,N-(bis-(2-dodecyloxyethyl))-N-methyl-1-ammonium bromide (PADELO), N,N,N-tris-(2-dodecyloxy)ethyl-N-(3-amino)propyl-ammonium bromide (PATELO), and N₁-(3-aminopropyl)((2-dodecyloxy)ethyl)-N₂-(2-dodecyloxy)ethyl-1-piperazinaminium bromide (GALOE-BP) can also be employed in the present invention.

Non-diether cationic lipids, such as DL-1,2-dioleoyl-3-dimethylaminopropyl-p-hydroxyethylammonium (DORI diester), 1-O-oleyl-2-oleoyl-3-dimethylaminopropyl-β-hydroxyethylammonium (DORI ester/ether), and their salts promote in vivo gene delivery. Preferred cationic lipids comprise groups attached via a heteroatom attached to the quaternary ammonium moiety in the head group. A glycyl spacer can connect the linker to the hydroxyl group.

Cationic lipids for use in certain embodiments of the present invention include DMRIE ((±)-N-(2-hydroxyethyl)-N,N-dimethyl-2-, 3-bis(tetradecyloxy)-1-propanaminium bromide), and GAP-DMORIE ((+)-N-(3-aminopropyl)-N,N-dimethyl-2,3-bis(syn-9-tetradeceneyloxy)-1-propanaminium bromide), as well as (±)-N,N-dimethyl-N-[2-(sperminecarboxamido)ethyl]-2,3-bis(dioleyloxy)-1-propanaminium pentahydrochloride (DOSPA), (±)-N-(2-aminoethyl)-N,N-dimethyl-2,3-bis(tetradecyloxy)-1-propanaminium bromide (β-aminoethyl-DMRIE or βAE-DMRIE) (Wheeler, et al., Biochim. Biophys. Acta 1280:1-11 (1996)), and (±)-N-(3-aminopropyl)-N,N-dimethyl-2,3-bis(dodecyloxy)-1-propanaminium bromide (GAP-DLRIE) (Wheeler, et al., Proc. Natl. Acad. Sci. USA 93:11454-11459 (1996)), which have been developed from DMRIE. Other examples of DMRIE-derived cationic lipids that are useful for the present invention are (±)-N-(3-aminopropyl)-N,N-dimethyl-2,3-(bis-decyloxy)-1-propanaminium bromide (GAP-DDRIE), (±)-N-(3-aminopropyl)-N,N-dimethyl-2,3-(bis-tetradecyloxy)-1-propanaminium bromide (GAP-DMRIE), (±)-N-((N″-methyl)-N′-ureyl)propyl-N,N-dimethyl-2,3-bis(tetradecyloxy)-1-propanaminium bromide (GMU-DMRIE), (±)-N-(2-hydroxyethyl)-N,N-dimethyl-2,3-bis(dodecyloxy)-1-propanaminium bromide (DLRIE), and (±)-N-(2-hydroxyethyl)-N,N-dimethyl-2,3-bis-([Z]-9-octadecenyloxy)propyl-1-propanaminium bromide (HP-DORIE).

A cationic lipid that may be used in concert with the compositions of the present invention is a “cytofectin.” As used herein, a “cytofectin” refers to a subset of cationic lipids which incorporate certain structural features including, but not limited to, a quaternary ammonium group and/or a hydrophobic region (usually with two or more alkyl chains), but which do not require amine protonation to develop a positive charge. Examples of cytofectins may be found, for example, in U.S. Pat. No. 5,861,397. Cytofectins that may be used in the present invention, include DMRIE ((±)-N-(2-hydroxyethyl)-N,N-dimethyl-2,3-bis(tetradecyloxy)-1-propanaminium bromide), GAP-DMORIE ((±)-N-(3-aminopropyl)-N,N-dimethyl-2,-3-bis(syn-9-tetradeceneyloxy)-1-propanaminium bromide), and GAP-DLRIE ((±)-N-(3-aminopropyl)-N,N-dimethyl-2,3-(bis-dodecyloxy)-1-propanaminium bromide).

The cationic lipid may be mixed with one or more co-lipids. The term “co-lipid” refers to any hydrophobic material which may be combined with the cationic lipid component and includes amphipathic lipids, such as phospholipids, and neutral lipids, such as cholesterol. Cationic lipids and co-lipids may be mixed or combined in a number of ways to produce a variety of non-covalently bonded macroscopic structures, including, for example, liposomes, multilamellar vesicles, unilamellar vesicles, micelles, and simple films. A preferred class of co-lipids are the zwitterionic phospholipids, which include the phosphatidylethanolamines and the phosphatidylcholines. Most preferably, the co-lipids are phosphatidylethanolamines, such as, for example, DOPE, DMPE and DPyPE. DOPE and DPyPE are particularly preferred. For immunization, the most preferred co-lipid is DPyPE, which comprises two phytanoyl substituents incorporated into the diacylphosphatidylethanolamine skeleton. The cationic lipid:co-lipid molar ratio may range from about 9:1 to about 1:9, or from about 4:1 to about 1:4, or from about 2:1 to about 1:2, or about 1:1. In order to maximize homogeneity, such cationic lipid and co-lipid components may be dissolved in a solvent such as chloroform, followed by evaporation of the cationic lipid/co-lipid solution under vacuum to dryness as a film on the inner surface of a glass vessel (e.g., a Rotovap round-bottomed flask). Upon suspension in an aqueous solvent, the amphipathic lipid component molecules self-assemble into homogenous lipid vesicles.

In some embodiments, such peptide or peptide construct(s) are combined with lipids by mixing, for example, a peptide-containing solution and a solution of cationic lipid:co-lipid liposomes. Preferably, the concentration of each of the constituent solutions is adjusted prior to mixing such that the desired final molecule that targets SR-BI/CLA-1/cationic lipid:co-lipid ratio and the desired final concentration of the molecule that targets SR-BI/CLA-1 will be obtained upon mixing the two solutions. For example, if the desired final solution is to be 2.5 mM sodium phosphate, the various components of the composition, e.g., plasmid DNA, cationic lipid:co-lipid liposomes, and any other desired auxiliary agents, transfection facilitating materials, or additives are each prepared in 2.5 mM sodium phosphate and then simply mixed to afford the desired complex. Alternatively, if the desired final solution is to be, e.g., 2.5 mM sodium phosphate, certain components of the composition, e.g., the auxiliary agent and/or cationic lipid:co-lipid liposomes, is prepared in a volume of water which is less than that of the final volume of the composition, and certain other components of the composition, e.g., the plasmid DNA, is prepared in a solution of sodium phosphate at a higher concentration than 2.5 mM, in a volume such that when the components in water are added to the components in the sodium phosphate solution, the final composition is in an aqueous solution of 2.5 mM sodium phosphate. The cationic lipid:co-lipid liposomes are preferably prepared by hydrating a thin film of the mixed lipid materials in an appropriate volume of aqueous solvent by vortex mixing at ambient temperatures for about 1 minute. The thin films are prepared by admixing chloroform solutions of the individual components to afford a desired molar solute ratio followed by aliquoting the desired volume of the solutions into a suitable container. The solvent is removed by evaporation, first with a stream of dry, inert gas (e.g. argon) followed by high vacuum treatment.

An uptake facilitating material can be used alone or in combination with one or more other uptake facilitating materials. Two or more uptake facilitating materials can be combined by chemical bonding (e.g, covalent and ionic such as in lipidated polylysine, PEGylated polylysine) (Toncheva, V., et al. (1998) “NOVEL VECTORS FOR GENE DELIVERY FORMED BY SELF-ASSEMBLY OF DNA WITH POLY(L-LYSINE) GRAFTED WITH HYDROPHILIC POLYMERS,” Biochim. Biophys. Acta 1380 (3):354-368), mechanical mixing (e.g., free moving materials in liquid or solid phase such as “polylysine+cationic lipids”) (Gao, X. et al. (1996) “POTENTIATION OF CATIONIC LIPOSOME-MEDIATED GENE DELIVERY BY POLYCATIONS,” Biochemistry 35:1027-1036); Trubetskoy, V. S., et al. (1992) “CATIONIC LIPOSOMES ENHANCE TARGETED DELIVERY AND EXPRESSION OF EXOGENOUS DNA MEDIATED BY N-TERMINAL MODIFIED POLY(L-LYSINE)-ANTIBODY CONJUGATE IN MOUSE LUNG ENDOTHELIAL CELLS,” Biochem. Biophys. Acta 1131:311-313), and aggregation (e.g., co-precipitation, gel forming such as in cationic lipids+poly-lactide co-galactide, and polylysine+gelatin).

Other hydrophobic and amphiphilic additives, such as, for example, sterols, fatty acids, gangliosides, glycolipids, lipopeptides, liposaccharides, neobees, niosomes, prostaglandins and sphingolipids, may also be included in the compositions of the present invention. In such compositions, these additives may be included in an amount between about 0.1 mol % and about 99.9 mol % (relative to total lipid). Preferably, these additives comprise about 1-50 mol % and, most preferably, about 2-25 mol %. Preferred additives include lipopeptides, liposaccharides and steroids.

In embodiments of the present invention in which the molecules that target SR-BI/CLA-1 are non-peptide compounds, such compounds can be formulated according to known methods for preparing such pharmaceutical compositions, whereby the substance to be delivered is combined with a pharmaceutically acceptable carrier vehicle.

Examples of non-peptide molecules that target SR-BI/CLA-1 and that may be employed in accordance with the methods of the present invention include:
Ezetimibe (Merck/Schering-Plough Pharmaceutical, SCH354009, etc.) (Simard, C. et al. (2003) “THE PHARMACOKINETICS OF EZETIMIBE,” Can J Clin Pharmacol. 10 Suppl A:13A-20A; Huff, M. W. et al. (2003) “DIETARY CHOLESTEROL, CHOLESTEROL ABSORPTION, POSTPRANDIAL LIPEMIA AND ATHEROSCLEROSIS,” Can J Clin Pharmacol. 10 Suppl A:26A-32A; 4: Lavoie, M. A. et al. (2003) “EZETIMIBE AND CHOLESTEROL ABSORPTION,” Can J Clin Pharmacol. 10 Suppl A:7A-12A; Davidson, M. H. (2003) “NEWER PHARMACEUTICAL AGENTS TO TREAT LIPID DISORDERS,” Curr Cardiol Rep. 5 (6):463-469 “EZETIMIBE FOR LOWERING BLOOD CHOLESTEROL,” Issues Emerg Health Technol. 49:1-4; Stroup, J. S. et al. (2003) “THE ANTILIPIDEMIC EFFECTS OF EZETIMIBE IN PATIENTS WITH DIABETES,” Diabetes Care. 26 (10):2958-9; Brown, W. V. (2003) “CHOLESTEROL ABSORPTION INHIBITORS: DEFINING NEW OPTIONS IN LIPID MANAGEMENT,” Clin Cardiol. 26 (6):259-64; Bruckert, E. et al. (2003) “PERSPECTIVES IN CHOLESTEROL-LOWERING THERAPY: THE ROLE OF EZETIMIBE, A NEW SELECTIVE INHIBIT OR OF INTESTINAL CHOLESTEROL ABSORPTION,” Circulation. 107 (25):3124-3128) and similar cholesterol lowering agents;
Enfuvirtide (Roche, Palo Alto, Calif.) (Ball, R. A. et al. (2003) “Injection site reactions with the HIV-1 fusion inhibitor enfuvirtide,” J Am Acad Dermatol. 49 (5):826-31; Cervia, J. S. et al. (2003) “Enfuvirtide (T-20): a novel human immunodeficiency virus type 1 fusion inhibitor,” Clin Infect Dis. 2003 Oct. 15; 37 (8): 1102-6. Epub 2003 Sep. 10; Koopmans, P. P. (2003) “Enfuvirtide, the first representative of a new class of drugs for the treatment of HIV infection: HIV fusion inhibitors,” Ned Tijdschr Geneeskd. 2003 Sep. 6; 147 (36):1726-9; Duffalo, M. L. et al. (2003) “Enfuvirtide: a novel agent for the treatment of HIV-1 infection,” Ann Pharmacother. 2003 October; 37 (10):1448-56; Veiga, S. et al. (2003) “Putative role of membranes in the HIV fusion inhibitor enfuvirtide mode of action at the molecular level, Biochem J. [Epub]; Meanwell, N. A. et al. (2003) “Inhibitors of the entry of HIV into host cells,” Curr Opin Drug Discov Devel. 6 (4):451-61; Ferranti, S. et al. (2003) “Enfuvirtide for prophylaxis against HIV Infection,” N Engl J Med. 2003 Aug. 21; 349 (8):815; (Anonymous) (2003) “Enfuvirtide (Fuzeon) for HIV Infection,” Med Lett Drugs Ther. 45 (1159):49-50) and similar viral fusion inhibitors;
Negatively charged lipids that bind to CLA-1 with Kd lower than 10⁻⁷ M;
Anti-SR-BI/CLA-1 antibody and fragments thereof that bind SR-BI/CLA-1 or CLA-1; and
Any chemical substance that binds to SR-BI/CLA-1 with a Kd lower than 10⁻⁷ M.

Suitable vehicles and their preparation are described, for example, in Remington's Pharmaceutical Sciences, 19^{th} Edition, A. R. Gennaro, Ed., Mack Publishing Co., Easton, Pa. (1995). The amount of such compounds included in such a composition depends on factors including the age and weight of the subject, the delivery method and route, the type of treatment desired, and the type of peptide or peptide construct or molecule being administered. In general, a composition of the present invention that includes such inhibitors will contain from about 1 ng to about 30 mg, and more preferably, from about 100 ng to about 10 mg of such inhibitor. Certain preferred compositions of the present invention may include about 1 ng of such inhibitor, about 5 ng of such inhibitor, about 10 ng of such inhibitor, about 50 ng of such inhibitor, about 100 ng of such inhibitor, about 500 ng of such inhibitor, about 1 μg of such inhibitor, about 5 μg of such inhibitor, about 10 μg of such inhibitor, about 50 μg of such inhibitor, about 100 μg of such inhibitor, about 150 μg of such inhibitor, about 200 μg of such inhibitor, about 250 μg of such inhibitor, about 300 μg of such inhibitor, about 350 μg of such inhibitor, about 400 μg of such inhibitor, about 450 μg of such inhibitor, about 500 μg of a polynucleotide, about 550 μg of such inhibitor, about 600 μg of such inhibitor, about 650 μg of such inhibitor, about 700 μg of such inhibitor, about 750 μg of such inhibitor, about 800 μg of such inhibitor, about 850 μg of a polynucleotide, about 900 μg of such inhibitor, about 950 μg of such inhibitor, about 1 mg of such inhibitor, about 5 μg of such inhibitor, about 10 mg of such inhibitor, about 15 mg of such inhibitor, about 20 mg of such inhibitor, about 25 mg of such inhibitor, or about 30 mg of such inhibitor.

Such compositions may be formulated into any of the various compositions and may be used in any of the methods disclosed herein. For aqueous compositions used in vivo, use of sterile pyrogen-free water is preferred. Such formulations will contain an effective amount of such inhibitor together with a suitable salt and/or auxiliary agent as disclosed herein, in order to prepare pharmaceutically acceptable compositions suitable for optimal administration to a vertebrate. Insoluble inhibitors may be solubilized in a weak acid or weak base, and then diluted to the desired volume, for example, with an aqueous solution of the present invention. The pH of the solution may be adjusted as appropriate. In addition, a pharmaceutically acceptable additive can be used to provide an appropriate osmolarity. Alternatively, lipids and lipid vehicles (as discussed above) may be used to facilitate the inhibitor administration. Other hydrophobic and amphiphilic additives, such as, for example, sterols, fatty acids, gangliosides, glycolipids, lipopeptides, liposaccharides, neobees, niosomes, prostaglandins and sphingolipids, may also be included in such compositions of the present invention. In such compositions, these additives may be included in an amount between about 0.1 mol % and about 99.9 mol % (relative to total lipid). Preferably, these additives comprise about 1-50 mol % and, most preferably, about 2-25 mol %. Preferred additives include lipopeptides, liposaccharides and steroids.

Pharmaceutical Compositions

The pharmaceutical composition of the present invention may be in the form of an emulsion, gel, solution, suspension, etc. In addition, the pharmaceutical composition can also contain pharmaceutically acceptable additives including, for example, diluents, binders, stabilizers, and preservatives. Administration of pharmaceutically acceptable salts of the peptides described herein is preferred. Such salts can be prepared from pharmaceutically acceptable non-toxic bases including organic bases and inorganic bases. Salts derived from inorganic bases include sodium, potassium, lithium, ammonium, calcium, magnesium, and the like. Salts derived from pharmaceutically acceptable organic non-toxic bases include salts of primary, secondary, and tertiary amines, basic amino acids, and the like. Preferred salts include but are not limited to sodium phosphate, sodium acetate, sodium bicarbonate, sodium sulfate, sodium pyruvate, potassium phosphate, potassium acetate, potassium bicarbonate, potassium sulfate, potassium pyruvate, disodium DL-α-glycerol-phosphate, and disodium glucose-6-phosphate. “Phosphate” salts of sodium or potassium can be either the monobasic form, e.g., NaHPO₄, or the dibasic form, e.g., Na₂HPO₄, but a mixture of the two, resulting in a desired pH, is most preferred. The most preferred salts are sodium phosphate or potassium phosphate. As used herein, the terms “sodium phosphate” or “potassium phosphate,” refer to a mixture of the dibasic and monobasic forms of each salt to present at a given pH.

Additional embodiments of the present invention are drawn to pharmaceutical compositions comprising one or more molecules that targets SR-BI/CLA-1 and an auxiliary agent. The present invention is further drawn to methods to use such compositions, methods of making such compositions, and pharmaceutical kits. As used herein, an “auxiliary agent” is a substance included in a composition for its ability to enhance, relative to a composition which is identical except for the inclusion of the auxiliary agent, the effectiveness of the SR-BI/CLA-1 targeting molecule. Auxiliary agents of the present invention include nonionic, anionic, cationic, or zwitterionic surfactant or detergents, with nonionic, anionic, cationic, or zwitterionic surfactant or detergents, with nonionic surfactant or detergents being preferred, chelators, protease inhibitors, agents that aggregate or condense nucleic acids, emulsifying or solubilizing agents, wetting agents, gel-forming agents, and buffers.

Suitable auxiliary agents include non-ionic detergents and surfactant such as poloxaners. Poloxamers are a series of non-ionic surfactant that are block copolymers of ethylene oxide and propylene oxide. The poly(oxyethylene) segment is hydrophillic and the poly(oxypropylene) segment is hydrophobic. The physical forms are liquids, pastes or solids. The molecular weight ranges from 1000 to greater than 16000. The basic structure of a poloxaner is HO—[CH₂CH₂O]ₓ—[CH₂CHO(CH₃)]_{y}—[CH₂CH₂O]ₓ—H, where x and y represent repeating units of ethylene oxide and propylene oxide respectively. Thus, the propylene oxide (PO) segment is sandwiched between two ethylene oxide (EO) segments, (EO-PO-EO). The number of x's and y's distinguishes individual poloxamers. If the ethylene oxide segment is sandwiched between two propylene oxide segments, (PO-EO-PO), then the resulting structure is a reverse poloxaner. The basic structure of a reverse poloxamer is HO—[CH(CH₃)CH₂O)]ₓ—[CH₂CH₂O]_{y}—[CH₂C—HO(CH₃)]ₓ—H.

Poloxmers that may be used in concert with the methods and compositions of the present invention include, but are not limited to commercially available poloxamers such as Pluronic® L121 (avg. MW: 4400), Pluronic® L101 (avg. MW: 3800), Pluronic® L81 (avg. MW: 2750), Pluronic® L61 (avg. MW: 2000), Pluronic® L31 (avg. MW: 1100), Pluronic® L122 (avg. MW: 5000), Pluronic® L92 (avg. MW: 3650), Pluronic® L72 (avg. MW: 2750), Pluronic® L62 (avg. MW: 2500), Pluronic® L42 (avg. MW: 1630), Pluronic® L63 (avg. MW: 2650), Pluronic® L43 (avg. MW: 1850), Pluronic® L64 (avg. MW: 2900), Pluronic® L44 (avg. MW: 2200), Pluronic® L35 (avg. MW: 1900), Pluronic® P123 (avg. MW: 5750), Pluronic® P103 (avg. MW: 4950), Pluronic® P104 (avg. MW: 5900), Pluronic® P84 (avg. MW: 4200), Pluronic® P105 (avg. MW: 6500), Pluronic® P85 (avg. MW: 4600), Pluronic® P75 (avg. MW: 4150), Pluronic® P65 (avg. MW: 3400), Pluronic® F127 (avg. MW: 12600), Pluronic® F98 (avg. MW: 13000), Pluronic® F87 (avg. MW: 7700), Pluronic® F77 (avg. MW: 6600), Pluronic® F 108 (avg. MW: 14600), Pluronic® F98 (avg. MW: 13000), Pluronic® F88 (avg. MW: 11400), Pluronic® F68 (avg. MW: 8400), and Pluronic® F38 (avg. MW: 4700).

Reverse poloxamers of the present invention include, but are not limited to Pluronic® R31R1 (avg. MW: 3250), Pluronic® R 25R1 (avg. MW: 2700), Pluronic® R17R1 (avg. MW: 1900), Pluronic® R31R2 (avg. MW: 3300), Pluronic® R25R2 (avg. MW: 3100), Pluronic® R17R2 (avg. MW: 2150), Pluronic® R12R3 (avg. MW: 1800), Pluronic® R31R4 (avg. MW: 4150), Pluronic® R25R4 (avg. MW: 3600), Pluronic® R22R4 (avg. MW: 3350), Pluronic® R17R4 (avg. MW: 3650), Pluronic® R25R5 (avg. MW: 4320), Pluronic® R10R5 (avg. MW: 1950), Pluronic® R25R8 (avg. MW: 8850), Pluronic® R17R8 (avg. MW: 7000), Pluronic® R10R8 (avg. MW: 4550).

Other commercially available poloxamers include compounds that are block copolymer of polyethylene and polypropylene glycol such as Synperonic® L121, Synperonic® L122, Synperonic® P104, Synperonic® P105, Synperonic® P123, Synperonic® P85, and Synperonic® P94; and compounds that are nonylphenyl polyethylene glycol such as Synperonic® NP10, Synperonic® NP30, and Synperonic® NP5.

Suitable auxiliary agents include non-ionic detergents and surfactants such as Pluronic® F68, Pluronic® F77, Pluronic® F108, Pluronic® F127, Pluronic® P65, Pluronic® P85, Pluronic® P103, Pluronic® P104, Pluronic® P105, Pluronic® P123, Pluronic® L31, Pluronic® L43, Pluronic® L44, Pluronic® L61, Pluronic® L62, Pluronic® L64, Pluronic® L81, Pluronic® L92, Pluronic® L101, Pluronic® L121, Pluronic® R17R4, Pluronic® R25R4, Pluronic® R25R2, IGEPAL CA 630®, NONIDET NP-40, Nonidet® P40, Tween-20®, Tween-80®, Triton X-100®, Triton X-114®, Thesit®; the anionic detergent sodium dodecyl sulfate (SDS); the sugar stachyose; the condensing agent DMSO; and the chelator/DNAse inhibitor EDTA. Even more preferred are the auxiliary agents Nonidet® P40, Triton X-100®, Pluronic® F68, Pluronic® F77, Pluronic® F108, Pluronic® P65, Pluronic® P103, Pluronic® L31, Pluronic® L44, Pluronic® L61, Pluronic® L64, Pluronic® L92, Pluronic® R17R4, Pluronic® R25R4 and Pluronic® R25R2. Most preferred auxiliary agent is Pluronic® R25R2.

Suitable concentrations of auxiliary agents of the present invention are disclosed in U.S. Patent Application Publication No. 20020019358 and PCT Publication WOO 80897A3. For example, in certain embodiments, pharmaceutical compositions of the present invention comprise about 5 ng to about 30 mg of a suitable peptide or a peptide construct, and/or a non-peptide molecule that targets SR-BI/CLA-1, and about 0.001% (w/v) to about 2.0% (w/v) of Pluronic® R 25R4, preferably about 0.002% (w/v) to about 1.0% (w/v) of Pluronic® R 25R4, more preferably about 0.01% (w/v) to about 0.01% (w/v) of Pluronic® R 25R4, with about 0.01% (w/v) of Pluronic® R 25R4 being the most preferred; about 0.001% (w/v) to about 2.0% (w/v) of Pluronic® R 25R2, preferably about 0.001% (w/v) to about 1.0% (w/v) of Pluronic® R 25R2, more preferably about 0.001% (w/v) to about 0.1% (w/v) of Pluronic® R 25R2, with about 0.01% (w/v) of Pluronic® R 25R2 being the most preferred.

Administration of the Pharmaceutical Compositions of the Present Invention

The pharmaceutical compositions of the present invention may be administered by any suitable means, for example, inhalation, or interdermally, intracavity (e.g., oral, vaginal, rectal, nasal, peritoneal, ventricular, or intestinal), intradermally, intramuscularly, intranasally, intraocularly, intraperitoneally, intrarectally, intratracheally, intravenously, orally, subcutaneously, transdermally, or transmucosally (i.e., across a mucous membrane) in a dose effective for the production of neutralizing antibody and resulting in protection from infection or disease. The present pharmaceutical compositions can generally be administered in the form of a spray for intranasal administration, or by nose drops, inhalants, swabs on tonsils, or a capsule, liquid, suspension or elixirs for oral administration. The pharmaceutical compositions may be in the form of single dose preparations or in multi-dose flasks. Reference is made to Remington's Pharmaceutical Sciences, Mack Publishing Co., Easton, Pa., Osol (ed.) (1980).

Administration can be into one or more tissues including but not limited to muscle, skin, brain, lung, liver, spleen, bone marrow, thymus, heart, e.g., myocardium, endocardium, and pericardium; lymph nodes, blood, bone, cartilage, pancreas, kidney, gall bladder, stomach, intestine, testis, ovary, uterus, rectum, nervous system, eye, gland, or connective tissue. Furthermore, in the methods of the present invention, the pharmaceutical compositions may be administered to any internal cavity of a mammal, including, but not limited to, the lungs, the mouth, the nasal cavity, the stomach, the peritoneal cavity, the intestine, any heart chamber, veins, arteries, capillaries, lymphatic cavities, the uterine cavity, the vaginal cavity, the rectal cavity, joint cavities, ventricles in brain, spinal canal in spinal cord, and the ocular cavities. Any mode of administration can be used so long as the mode results prophylactic or therapeutic efficacy. Methods to detect such a response include serological methods, e.g., western blotting, staining tissue sections by immunohistochemical methods, and measuring the activity of the peptide. Pharmaceutical DNA compositions and methods of their manufacture and delivery that may be used in accordance with the present invention are disclosed in U.S. Pat. Nos. 5,589,466; 5,620,896; 5,641,665; 5,703,055; 5,707,812; 5,846,946; 5,861,397; 5,891,718; 6,022,874; 6,147,055; 6,214,804; 6,228,844; 6,399,588; 6,413,942; 6,451,769, European Patent Documents EP1165140A2; EP1006796A1 and EP0929536A1; and PCT Patent Publications WO00/57917; WO00/73263; WO01/09303; WO03/028632; WO94/29469; WO95/29703; and WO98/14439.

Administration may be by needle injection, catheter infusion, biolistic injectors, particle accelerators (e.g., “gene guns” or pneumatic “needleless” injectors) Med-E-Jet (Vahising, H., et al. (1994) “IMMUNIZATION WITH PLASMID DNA USING A PNEUMATIC GUN,” J. Immunol. Methods 171:11-22), Pigjet (Schrijver, R. S. et al. (1997) “IMMUNIZATION OF CATTLE WITH A BHV1 VECTOR VACCINE OR A DNA VACCINE BOTH CODING FOR THE G PROTEIN OF BRSV,” Vaccine 15:1908-1916), Biojector (Davis, H. L. et al. (1994) “DIRECT GENE TRANSFER IN SKELETAL MUSCLE: PLASMID DNA-BASED IMMUNIZATION AGAINST THE HEPATITIS B VIRUS SURFACE ANTIGEN,” Vaccine 12:1503-1509; Gramzinski, R., et al. (1998) “IMMUNE RESPONSE To A HEPATITIS B DNA VACCINE IN AOTUS MONKEYS: A COMPARISON OF VACCINE FORMULATION, ROUTE, AND METHOD OF ADMINISTRATION,” Mol Med 4:109-118), AdvantaJet (Lindmayer, I., et al. (1986) “DEVELOPMENT OF NEW JET INJECTOR FOR INSULIN THERAPY,” Diabetes Care 9:294-297), Medi-jector (Martins, J. K. et al. (1979) “MEDIJECTOR-A NEW METHOD OF CORTICOSTEROID-ANESTHETIC DELIVERY,” J. Occup. Med. 21:821-824), gelfoam sponge depots, other commercially available depot materials (e.g., hydrogels), osmotic pumps (e.g., Alza minipumps), oral or suppositorial solid (tablet or pill) pharmaceutical formulations, topical skin creams, and decanting, use of peptide-coated suture (analogous to the polynucleotide-coated suture disclosed by Qin, J. Y. et al. (1999) (“GENE SUTURE—A NOVEL METHOD FOR INTRAMUSCULAR GENE TRANSFER AND ITS APPLICATION IN HYPERTENSION THERAPY,” Life Sciences 65:2193-2203)) or topical applications during surgery.

Preferably, the pharmaceutical composition is delivered to the interstitial space of a tissue. “Interstitial space” comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels.

The compositions of the present invention can be lyophilized to produce pharmaceutical compositions in a dried form for ease in transportation and storage. The pharmaceutical compositions of the present invention may be stored in a sealed vial, ampule or the like. In the case where the pharmaceutical composition is in a dried form, the composition is dissolved or suspended (e.g., in sterilized distilled water) before administration. An inert carrier such as saline or phosphate buffered saline or any such carrier in which the pharmaceutical compositions has suitable solubility, may be used.

Further, the pharmaceutical compositions may be prepared in the form of a mixed composition that contains one or more additional constituents so long as such additional constituents do not interfere with the effectiveness of the SR-BI/CLA-1 targeting molecule and the side effects and adverse reactions are not increased additively or synergistically. The pharmaceutical compositions of the present invention can be associated with chemical moieties which may improve the composition's solubility, absorption, biological half life, etc. The moieties may alternatively decrease the toxicity of the pharmaceutical compositions, eliminate or attenuate any undesirable side effect of the pharmaceutical compositions, etc. Moieties capable of mediating such effects are disclosed in Remington's Pharmaceutical Sciences (1995). Procedures for coupling such moieties to a molecule are well known in the art.

Determining an effective amount of a composition depends upon a number of factors including, for example, the chemical structure and biological activity of the substance, the age and weight of the subject, the precise condition requiring treatment and its severity, and the route of administration. Based on the above factors, determining the precise amount, number of doses, and timing of doses are within the ordinary skill in the art and will be readily determined by the attending physician or veterinarian.

In one embodiment, the pharmaceutical compositions of the present invention are administered free from association with liposomal formulations, charged lipids, or transfection-facilitating viral particles. In another embodiment, the compositions of the present invention are administered free from association with any delivery vehicle now known in the art that can facilitate entry into cells.

As used herein, “ex vivo” cells are cells into which the pharmaceutical compositions is introduced, for example, by transfection, lipofection, electroporation, bombardment, or microinjection. The cells containing the pharmaceutical compositions are then administered in vivo into mammalian tissue (see, for example, see Belldegrun, A., et al. (1993) “HUMAN RENAL CARCINOMA LINE TRANSFECTED WITH INTERLEUKIN-2 AND/OR INTERFERON ALPHA GENE(S): IMPLICATIONS FOR LIVE CANCER VACCINES,” J. Natl. Cancer Inst. 85: 207-216; Ferrantini, M. et al. (1993) “ALPHA 1-INTERFERON GENE TRANSFER INTO METASTATIC FRIEND LEUKEMIA CELLS ABROGATED TUMORIGENICITY IN IMMUNOCOMPETENT MICE: ANTITUMOR THERAPY BY MEANS OF INTERFERON-PRODUCING CELLS,” Cancer Research 53:1107-1112; Ferrantini, M. et al. (1994) “IFN-ALPHA 1 GENE EXPRESSION INTO A METASTATIC MURINE ADENOCARCINOMA (TS/A) RESULTS IN CD8⁺ T CELL-MEDIATED TUMOR REJECTION AND DEVELOPMENT OF ANTITUMOR IMMUNITY. COMPARATIVE STUDIES WITH IFN-GAMMA-PRODUCING TS/A CELLS,” J. Immunology 153:4604-4615; Kaido, T. et al. (1995) “IFN-ALPHA 1 GENE TRANSFECTION COMPLETELY ABOLISHES THE TUMORIGENICITY OF MURINE B16 MELANOMA CELLS IN ALLOGENEIC DBA/2 MICE AND DECREASES THEIR TUMORIGENICITY IN SYNGENEIC C57BL/6 MICE,” Int. J. Cancer 60: 221-229; Ogura, H. et al. (1990) “IMPLANTATION OF GENETICALLY MANIPULATED FIBROBLASTS INTO MICE AS ANTITUMOR ALPHA-INTERFERON THERAPY,” Cancer Research 50:5102-5106; Santodonato, L. et al. (1996) “CURE OF MICE WITH ESTABLISHED METASTATIC FRIEND LEUKEMIA CELL TUMORS BY A COMBINED THERAPY WITH TUMOR CELLS EXPRESSING BOTH INTERFERON-ALPHA 1 AND HERPES SIMPLEX THYMIDINE KINASE FOLLOWED BY GANCICLOVIR,” Human Gene Therapy 7: 1-10; Santodonato, L., et al. (1997) “LOCAL AND SYSTEMIC ANTITUMOR RESPONSE AFTER COMBINED THERAPY OF MOUSE METASTATIC TUMORS WITH TUMOR CELLS EXPRESSING IFN-ALPHA AND HSVTK: PERSPECTIVES FOR THE GENERATION OF CANCER VACCNES,” Gene Therapy 4:1246-1255; and Zhang, J. F. et al. (1996) “GENE THERAPY WITH AN ADENO-ASSOCIATED VIRUS CARRYING AN INTERFERON GENE RESULTS IN TUMOR GROWTH SUPPRESSION AND REGRESSION,” Cancer Gene Therapy 3:31-38.

In the “local delivery” embodiment of the present invention, a pharmaceutical composition is administered in vivo, such that the SR-BI/CLA-1 targeting molecule is incorporated into the local cells at the site of administration. The pharmaceutical compositions can be administered either within ex vivo cells or free of ex vivo cells or ex vivo cellular material. Preferably, the peptide construct is administered free of ex vivo cells or ex vivo cellular material.

The pharmaceutical compositions can be solubilized in a buffer prior to administration. Suitable buffers include, for example, phosphate buffered saline (PBS), normal saline, Tris buffer, and sodium phosphate vehicle (100-150 mM preferred). Insoluble peptides can be solubilized in a weak acid or base, and then diluted to the desired volume with a neutral buffer such as PBS. The pH of the buffer is suitably adjusted, and moreover, a pharmaceutically acceptable additive can be used in the buffer to provide an appropriate osmolarity within the lipid vesicle. Preferred salt solutions and auxiliary agents are disclosed herein.

A systemic delivery embodiment is particularly preferred for treating non-localized disease conditions. A local delivery embodiment can be particularly useful for treating disease conditions that might be responsive to high local concentration. When advantageous, systemic and local delivery can be combined. U.S. Pat. Nos. 5,589,466, 5,693,622, 5,580,859, 5,703,055, and PCT publication WO94/29469 provide methods for delivering compositions comprising naked DNA, or DNA cationic lipid complexes to mammals.

Compositions used in of the present invention can be formulated according to known methods. Suitable preparation methods are described, for example, in Remington's Pharmaceutical Sciences, 19^{th} Edition, A. R. Gennaro, ed., Mack Publishing Co., Easton, Pa. (1995), incorporated herein by reference in its entireties. Although the composition is preferably administered as an aqueous solution, it can be formulated as an emulsion, gel, solution, suspension, lyophilized form, or any other form known in the art. According to the present invention, if the composition is formulated other than as an aqueous solution, it will require resuspension in an aqueous solution prior to administration. In addition, the composition may contain pharmaceutically acceptable additives including, for example, diluents, binders, stabilizers, and preservatives.

The present invention also provides kits for use in treating sepsis and/or inflammation comprising an administration means and a container means containing a pharmaceutical composition of the present invention. Preferably, the container in which the composition is packaged prior to use will comprise a hermetically sealed container enclosing an amount of the lyophilized formulation or a solution containing the formulation suitable for a pharmaceutically effective dose thereof, or multiples of an effective dose. The composition is packaged in a sterile container, and the hermetically sealed container is designed to preserve sterility of the pharmaceutical formulation until use. Optionally, the container can be associated with administration means and/or instruction for use.

Having now generally described the invention, the same will be more readily understood through reference to the following examples, which are provided by way of illustration, and are not intended to be limiting of the present invention, unless specified.

### Example 1

### Lipopolysaccharide Down Regulates Both Scavenger Receptor B1 And ATP Binding Cassette Transporter A1 In RAW Cells

### Materials and Methods

Cell culture and treatment. RAW 264.7 mouse monocyte-macrophages (ATCC TIE 71) are grown in 12-well plates in Dulbecco modified Eagle medium (DMEM) supplemented with 10% fetal bovine serum, penicillin (100 U/ml), and streptomycin (100 μg/ml) in a humidified atmosphere containing 5% CO₂ and 95% air at 37° C. The experiments are carried out on the confluent monolayers in serum-free DMEM. Cells are treated with the following LPS preparations at 10 ng/ml for 24 h: full-length LPS from Escherichia coli serotype 0111:B4 (Sigma Chemical Co., St. Louis, Mo.) or Re595 mutant LPS, diphosphoryllipid A (DPLA), or monophosphoryllipid A (MPLA) (from Salmonella enterica serovar Minnesota; Sigma Chemical Co.). The serine protease inhibitor tosylphenyl chloromethyl ketone (TPCK) or tosyllysyl chloromethyl ketone (TLCK) (Sigma Chemical Co.) was added to the cells at 20 mM 2 h before the LPS treatment and is present in the experimental medium simultaneously with LPS for the next 22 h.

Western immunoblot analysis. At the end of the incubation the cells are harvested, washed with phosphate-buffered saline (PBS) (pH 7.4) containing 5 mM EDTA and 1 mM phenylmethylsulfonyl fluoride, and incubated in the same buffer containing 2% Triton X-100 for 15 min at 4° C. Following lysis, cell debris was removed by the centrifugation (12,000×g, 4° C., 10 min). The supernatants are delipidated by addition of a mixture of methanol and chloroform (4:1) and consequent centrifugation at 12,000×g for 10 min at 4° C. The pellets are dissolved in the sample buffer and heated to 82° C. (15 min). The aliquots of samples are then applied to 4 to 20% precast gels (Invitrogen, Carlsbad, Calif.) and separated by sodium dodecyl sulfate-polyacrylamide gel electrophoresis. Proteins are electrophoretically transferred to nitrocellulose, and the membranes are incubated with TBS (200 mM Tris-HCl, 150 mM NaCl, 5% nonfat dry milk) blocking solution for 1 h at room temperature. Membranes are incubated with rabbit polyclonal anti-SR-BI/CLA-1 antibodies (diluted 1:1,000) (Novus, Littleton, Colo.) and mouse monoclonal anti-[3-actin antibodies (1:10,000) (Sigma Chemical Co.) overnight, washed three times with TBS containing 0.1% Tween 20, and then incubated with goat anti-rabbit or anti-mouse antiserum (1:10,000) conjugated to alkaline phosphatase for 1 h at room temperature. Quantitative comparison of the bands is performed by densitometry.

RNA isolation and cDNA preparation. The total RNA of cultured cells is isolated and purified using Trizol reagent (Gibco BRL, Grand Island, N.Y.) according to the manufacturer's protocol. The concentration and quality of RNA were determined by UV absorbance at 260 and 280 nm. To prepare cDNA, total RNA (3 μg) is added to a mixture containing the following: 6 μl of 5× first-strand buffer (75 mM KCl, 50 mM Tris-HCl [pH 8.3], 3 mM MgCl₂), 1.5 μl of deoxynucleoside triphosphates (10 mM [each] dATP, dCTP, dTTP, and dGTP), 1.5 μl of 0.0156 U (0.4 μg) of random hexamers, and 0.2 U of Moloney murine leukemia virus reverse transcriptase (all from Gibco BRL, Gaithersburg, Md.); 0.004 U of RNasin (Promega, Madison, Wis.); and RNase-free water to a final volume of 30 μl per 3 μg of cDNA. Samples are incubated at 37° C. for 60 min. Preliminary experiments are undertaken to achieve optimal conditions for amplifying mRNA for each of the gene products.

Reverse transcription-PCR (RT-PCR). A mixture of 37.2 μl of RNase-free water, 5 μl of 10× reaction buffer, and 0.5 μl (5 U) of AmpliTaq Gold DNA polymerase (Applied Biosystems, Roche Molecular Systems, Inc., Branchburg, N.J.), sense and antisense primers (1 μl each), and 0.3 μl (3 μCi) of [α-³²P]dCTP (Amersham Pharmacia Biotech, San Diego, Calif.) is vortexed, and 46 μl is aliquoted into each tube, containing 4 μl of cDNA, and overlaid with 50 μl of mineral oil (Sigma Chemical Co.). cDNA is amplified in a Perkin-Elmer (Norwalk, Conn.) System 2400 DNA thermal cycler, with denaturation for 1 min at 94° C., annealing for 1 min at 50° C., and extension for 2 min at 72° C. for GADPH (glyceraldehyde-3-phosphate dehydrogenase) (19 cycles), ABCA1 (28 cycles), SR-BI/CLA-1, and interleukin-1 [3 (IL-1β (27 cycles). The primers used in these analyses are as follows: GADPH, (SEQ ID NO:1) 5′-GTCTTCACCACCATGGAGAAG-3′ and (SEQ ID NO:2) 5′-GCTTCACCACCTTCTTGATGTCATC-3′; SR-BI/CLA-1, (SEQ ID NO:3) 5′-CCA CCCAACGAA GGC TTC TGC-3′ and (SEQ ID NO:4) 5′-CTG AAT GGC CTC CTT ATC C-3′; ABCA1, (SEQ ID NO:5) 5′-CAA CTA CAA AGC CCT CTT TG-3′ and (SEQ ID NO:6) 5′-CTT GGC TGT TCT CCA TGA AG-3′; and IL-1β, (SEQ ID NO:7) 5′-CTG AAA GCT CTC CAC CTC-3′ and (SEQ ID NO:8) 5′-GTG CTG ATG TAC CAG TTG-3′.

For the densitometry analysis, the intensities of the bands are measured with the Gel-Pro Analyzer 3.0 computer program and normalized with GADPH intensity.

125I-HDL binding assay. 125I-HDL binding experiments are performed as described by Bocharov, A. V. et al. (2001) (“CHARACTERIZATION OF A 95 KDA HIGH AFFINITY HUMAN HIGH DENSITY LIPOPROTEIN-BINDING PROTEIN,” Biochemistry 140:4407-4416). RAW cells cultured in 12-well plates are incubated with 1 μg of LPS per ml for 24 h. After three subsequent washes with PBS, the cells were chilled on ice and binding of 125I-HDL (5 μg/ml) is determined in the absence (total binding) or presence (nonspecific binding) of a 50-fold excess of unlabeled ligand. Radioactivity was counted in an LKB-Wallac Ultragamma counter. The protein contents of samples are determined after hydrolysis in 0.1 N NaOH followed by neutralization with 0.1 N HCl by the method of Bradford, M. M. (1976) (“A RAPID AND SENSITIVE METHOD FOR THE QUANTITATION OF MICROGRAM QUANTITIES OF PROTEIN UTILIZING THE PRINCIPLE OF PROTEIN-DYE BINDING,” Anal. Biochem. 72:248-254).

Cholesterol efflux studies. The cholesterol efflux assay is performed essentially according to the protocol described by Marcil, M. et al. (1999) (“CELLULAR CHOLESTEROL TRANSPORT AND EFFLUX FROM FIBROBLASTS ARE ABNORMAL IN SUBJECTS WITH FAMIILIAL HDL DEFICIENCY,” Arterioscier. Thromb. Vasco Biol. 19:159-169). At 70 to 80% confluence, cholesterol-loaded cells grown on 24-well plates are incubated with DMEM containing 1 μCi of [1,2-³H]cholesterol (50 Ci/mmol; Amersham Pharmacia Biotech, Piscataway, N.J.) per ml for 48 h. Cells are washed three times with PBS containing 1 mg of bovine serum albumin per ml (PBS-BSA), and cellular cholesterol pools are allowed to equilibrate for another 24 h in DMEM containing 1 mg of BSA per ml (DMEM-BSA) and increasing doses (0, 10, 30, 100, 300, and 1,000 ng/ml) of LPS. After intensive washing of cells with PBSBSA, efflux studies (24 h) are carried out using 100 μg of HDL (Calbiochem, San Diego, Calif.) per ml prepared in DMEM-BSA as the cholesterol acceptor. After the efflux period, medium is collected and centrifuged (10,000×g for 5 min), and radioactivity is counted by liquid scintillation counting. The residual radioactivity in the cell fraction is determined after an overnight extraction with hexane-isopropanol (3:2). The percent efflux is calculated by dividing the radioactive counts in the efflux medium by the sum of the radioactive counts in the medium and the cell fraction. DMEM-BSA is used as the blank, the radioactive counts in which are subtracted from the counts obtained in the presence of a cholesterol acceptor.

Statistical analysis. All results are reproduced in at least two independent experiments. The results are presented as the means of triplicate determinations :+: standard deviations. Comparisons between groups of data are performed by a Student's t test. P values of less than 0.05 are considered statistically significant.

### Results of Analysis

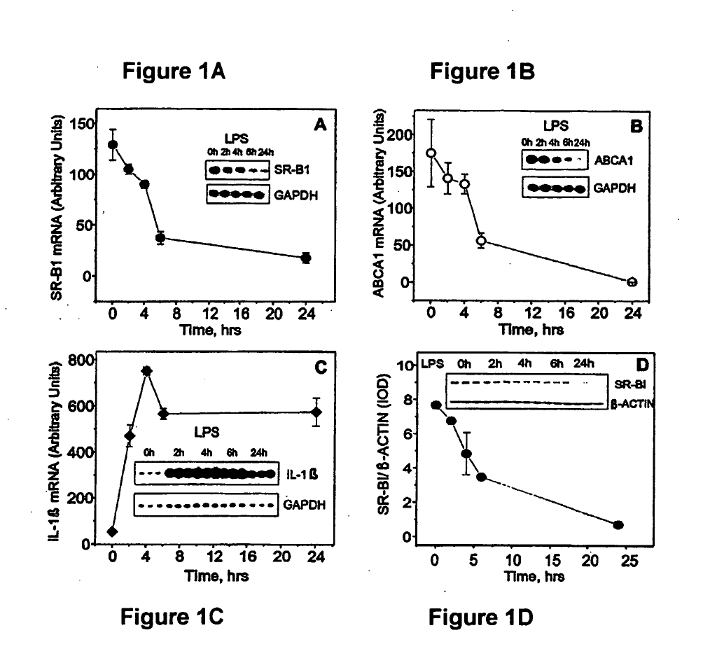

Time courses of ABCA1, SR-BI/CLA-1, and IL-1β mRNA biosynthesis and SR-BI/CLA-1 protein expression in response to LPS exposure. To study the kinetics of LPS effects upon the mRNA levels of SR-BI/CLA-1, ABCA1, and IL-1β (IL-1β is used as a well-established LPS-up-regulated cytokine), RAW cells are exposed to LPS (1 μg/ml) for increasing periods of time (FIGS. 1A, 1B, 1C, and 1D). The decrease in both SR-BI/CLA-1 and ABCA1 gene expression is detectable as early as 4 h after exposure to LPS and reaches its maximum after 24 h. The LPS-induced increase of IL-1β gene expression has more rapid kinetics and reaches its maximum after 4 h of LPS exposure, remaining still significantly elevated versus the control level at 24 h. SR-BI/CLA-1 protein production is decreased by 50% after 6 h (FIG. 1D). This demonstrates that in addition to the widely known ability of LPS to induce the expression of proinflammatory cytokine genes, LPS is a powerful inhibitor of SR-BI/CLA-1 and ABCA1 expression in murine monocytes.

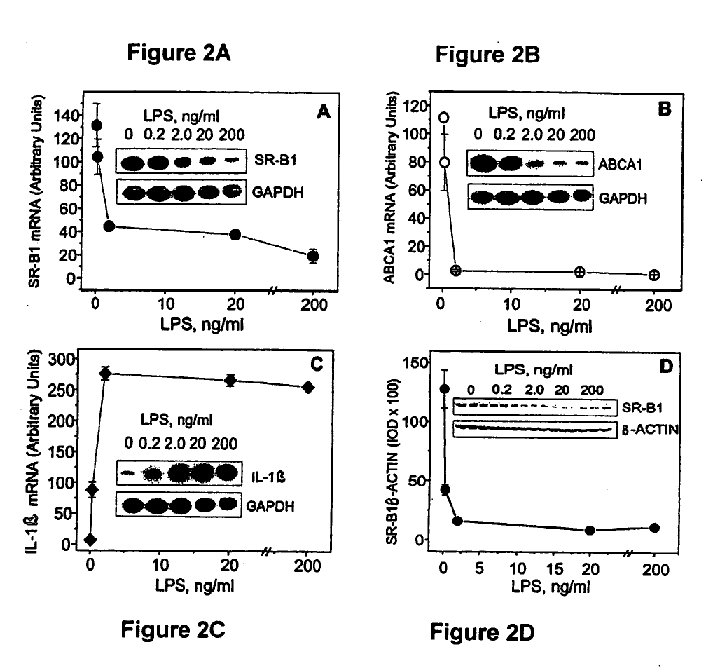

Dose-dependent response of LPS-sensitive genes to LPS. To study the dose dependence of the LPS effect upon the SR-BI/CLA-1, ABCA1, and IL-1β mRNAs as well as on SR-BI/CLA-1 protein expression, RAW cells are exposed to the increasing concentrations of LPS (0.2 to 200 ng/ml) for 24 h. As shown in FIGS. 2A, 2B and 2C, LPS induced a dose-dependent increase of IL-1β mRNA or decrease of ABCA1 and SR-BI/CLA-1 mRNA with very similar patterns. SR-BI/CLA-1 gene expression is significantly reduced, but less so than ABCA1 expression. SR-BI/CLA-1 protein expression demonstrates a pattern of LPS inhibition (FIG. 2D) similar to that for the mRNA.

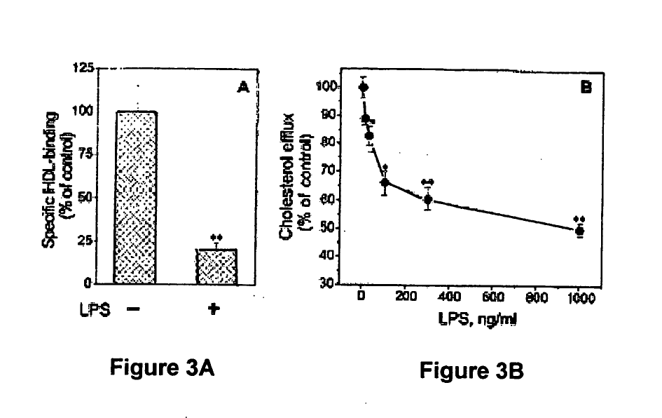

LPS-mediated suppression of ¹²⁵I-HDL binding and cholesterol efflux to HDL. In order to determine if there is any correlation between LPS-mediated down regulation of both HDL binding protein mRNA and their physiological function, ¹²⁵I-HDL binding assay are conducted after the incubation of cells with LPS (1 μg/ml) for 24 h. As a result of pretreatment with LPS, a significant decrease of the specific ¹²⁵IHDL binding (FIG. 3A), to 20% of the control level is observed. Next cholesterol efflux experiments are performed, using HDL (100 μg/ml) as the cholesterol acceptor. Following preincubation with increasing LPS concentrations (0 to 1,000 ng/ml), one can detect a dose-dependent inhibitory effect upon the HDL mediated [3H]cholesterol efflux in cultured RAW cells (FIG. 3B). The highest dose of the LPS (1 μg/ml) results in an approximately 50% decrease of [3H]cholesterol efflux to HDL compared to that in untreated cells.

Comparison of the abilities of different LPS preparations to modulate the expression of LPS-responsive genes. Although the lipid A component is proposed as being the active portion for LPS bioactivities, a variety of lipid A partial structures and analogues are reported to have different properties. The complete form of LPS from E. coli (serotype 0111:B4), LPS of the Re595 mutant of Salmonella enterica serovar Minnesota (lacking O antigen and outer core polysaccharide), Re595 DPLA (lacking O antigen and outer and inner core polysaccharides), and Re595 MPLA (lacking O antigen, outer and inner polysaccharides, and one phosphoryl group) are evaluated for their ability to affect IL-1f3, ABCA1, and SR-BI/CLA-1 mRNA expression.

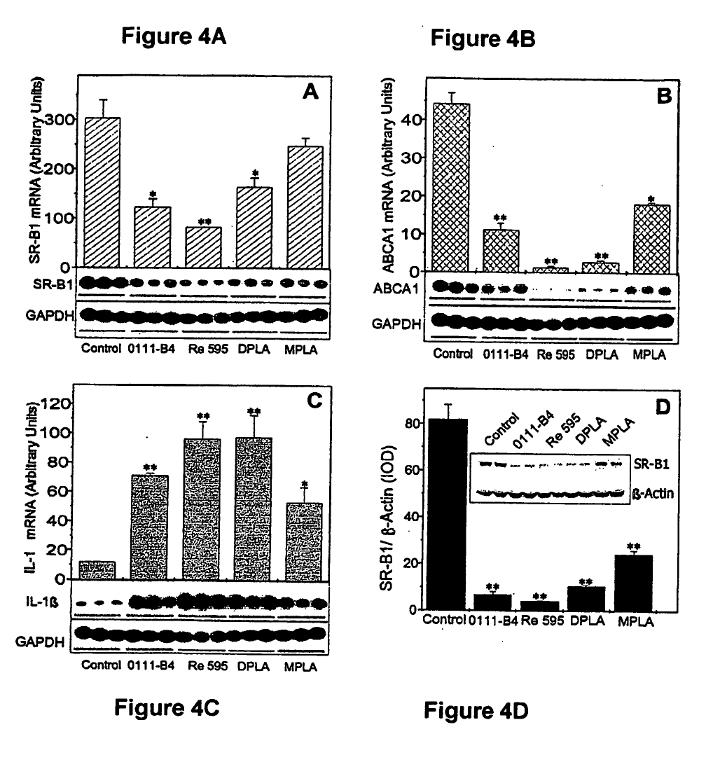

As shown in FIGS. 4A, 4B, 4C and 4D, MPLA exhibits decreased activity compared to the other LPS derivatives, which markedly (7- to 10-fold) elevate the IL-1β mRNA level and significantly inhibits ABCA1 (by 75 to 80%) and SR-BI/CLA-1 (50%) mRNA biosynthesis at a concentration of 10 ng/ml after 24 h. Under the same conditions, SR-BI/CLA-1 protein expression demonstrates very similar relative responses to the LPS analogues (FIG. 4D). However, when a concentration of 1 μg/ml was used, no significant differences are observed between the LPS derivatives in their ability to suppress SR-BI/CLA-1 production. These results indicate that for the suppressive effect of the LPS preparations on SR-BI/CLA-1 and ABCA1 expression as well as on SR-BI/CLA-1 protein expression, the relative activities are LPS (Re595)>LPS (0111:B4)>DPLA>MPLA. For IL-1β mRNA up regulation, a statistically significant difference can be observed only for MPLA, which demonstrates approximately one-half the potency for IL-1β gene induction of Re595 LPS or DPLA.

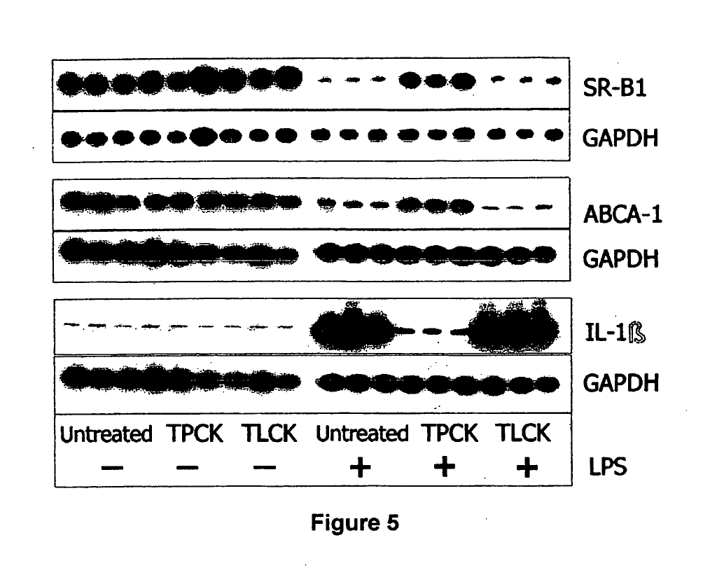

Effect of NF-KB inhibitors on LPS-modulated gene expression. NF-KB is a major transcription factor that up regulates proinflammatory cytokine expression (Baenerle, P. A. (1997) “NF-KAPPA B AS A FREQUENT TARGET FOR IMMUNOSUPPRESSIVE AND ANTI-INFLAMMATORY MOLECULES,” Adv. Immunol. 65:111-137; Marcil, M. et al. (1999) “CELLULAR CHOLESTEROL TRANSPORT AND EFFLUX FROM FIBROBLASTS ARE ABNORMAL IN SUBJECTS WITH FAMILIAL HDL DEFICIENCY,” Arterioscler. Thromb. Vasco Biol. 19:159-169). To examine whether the suppressive effect of LPS on SR-BI/CLA-1 and ABCA1 expression is mediated through of NF-KB, RAW cells are incubated with TPCK (chymotrypsin-like serine protease inhibitor, a potent inhibitor of NF-KB activation) or TLCK (a structural analog of the TPCK that lacks NF-KB inhibitor activity) in the presence of LPS (10 ng/ml) for 24 h. TPCK treatment effectively prevented the LPS-induced SR-BI/CLA-1 and ABCA1 mRNA level decrease as well as the increase of IL-1f3 (FIG. 5). At the same time, the negative control, TLCK, is not able to reverse these LPS-mediated effects. These data demonstrate that inhibition of NF-KB is sufficient to block LPS induced suppression of both the ABCA1 and SR-BI/CLA-1 genes, indicating the involvement of NF-KB activation as the mechanism in this process.

### Conclusions

In atherosclerosis, both clinical and biochemical evidence strongly suggests that lesion development can be accelerated by local actions of inflammatory cytokines on endothelial cells (Hansson, G. K. (1997) “CELL-MEDIATED IMMUNITY IN ATHEROSCLEROSIS,” Curr. Opin. Lipidol. 8:301-311; Mantovani, A. et al. (1997) “CYTOKINEACTIVATION OF ENDOTHELIAL CELLS: NEW MOLECULES FOR AN OLD PARADIGM,” Thromb. Haemost. 78:406-414). Several reports link atherosclerosis with infection and inflammation. For example, a higher incidence of coronary artery heart disease occurs in patients with Chlamydia pneumoniae and cytomegalovirus infections, and these microorganisms have been detected in atherosclerosis plaques (Chiu, B. et al. (1997) “CHLAMYDIA PNEUMONIAE, CYTOMEGALOVIRUS, AND HERPES SIMPLEX VIRUS IN ATHEROSCLEROSIS OF CAROTID ARTERY,” Circulation 96:2144-2148; Mendall, M. A. (1995) “CHLAMYDIA PNEUMONIAE: RISK FACTORS FOR SEROPOSITIVITY AND ASSOCIATION WITH CORONARY HEART DISEASE,” J. Infect. 30:121-128). In addition, LPS can induce oxidative modification of low-density lipoproteins (Cathcart, M. K. et al. (1989) “SUPEROXIDE ANION PARTICIPATION IN HUMAN MONOCYTE-MEDIATED OXIDATION OF LOW-DENSITY LIPOPROTEIN AND CONVERSION OF LOW-DENSITY LIPOPROTEIN To A CYTOTOXIN,” J. Immunol. 142:1963-1969), a major mediator in atherogenesis (Lopes-Virella, M. F. (1993) “INTERACTIONS BETWEEN BACTERIAL LIPOPOLYSACCHARIDES AND SERUM LIPOPROTEINS AND THEIR POSSIBLE ROLE IN CORONARY HEART DISEASE,” Eur. Heart J. 14:118-124). Recent data obtained by Kalayoglu and Byrne (Kalayoglu, M. V. et al. (1998) “A CHLAMYDIA PNEUMONIAE COMPONENT THAT INDUCES MACROPHAGE FOAM CELL FORMATION IS CHLAMYDIAL LIPOPOLYSACCHARIDE,” Infect. Immun. 66:5067-5072) indicate that chlamydial LPS induces macrophage foam cell formation and suggest that infected macrophages chronically exposed to chlamydial LPS may accumulate excess cholesterol, contributing to atheroma development.

No previous data suggest a modulating role of low-dose LPS exposure on the expression of two key HDL binding proteins, ABCA1 and SR-BI/CLA-1. The present study demonstrates that LPS is able to negatively regulate the SR-BI/CLA-1 and ABCA1 mRNA levels in RAW cells. The expression of the SR-BI/CLA-1 protein was similarly suppressed. In the above-reported study the LPS effect upon ABCA1 protein expression was not investigated by the immunoblotting assay, as noable to detect ABCA1 in the samples under standard conditions. The choice of murine macrophages for the above-reported study was determined by the fact that the macrophage-like RAW 264.7 cell line is a well-characterized model system in terms of LPS-induced macrophage activation resulting in the proinflammatory cell response (Gao, J. J. et al. (2001) “BACTERIAL DNA AND LIPOPOLYSACCHARIDE INDUCE SYNERGISTIC PRODUCTION OF TNF-ALPHA THROUGH A POST-TRANSCRIPTIONAL MECHANISM,” J. Immunol. 166:6855-6860; Scott, M. G. et al. (2000) “AN ALPHA-HELICAL CATIONIC ANTIMICROBIAL PEPTIDE SELECTIVELY MODULATES MACROPHAGE RESPONSES To LIPOPOLYSACCHARIDE AND DIRECTLY ALTERS MACROPHAGE GENE EXPRESSION,” J. Immunol. 165:3358-3365). In addition, this cell line is a model widely used in numerous macrophage regulatory studies of cholesterol efflux (Schwartz, K. et al. (2000) “ABC1 GENE EXPRESSION AND APOA-I-MEDIATED CHOLESTEROL EFFLUX ARE REGULATED BY LXR,” Biochem. Biophys. Res. Commun. 274:794-802; Suzuki, S. et al. (2000) “ENHANCEMENT OF THE CAMP-INDUCED APOLIPOPROTEIN-MEDIATED CELLULAR LIPID RELEASE BY CALMODULIN INHIBITORS W7 AND W5 FROM RAW 264 MOUSE MACROPHAGE CELL LINE CELLS,” J. Cardiovasc. Pharmacol. 36:609-616). At the same time, this cell line (like other models) has potential limitations, as the regulatory pathways of gene expression may reflect differences of species or transformation. The above-reported findings concerning the inhibitory effect of LPS on SR-BI/CLA-1 gene expression as well as on protein expression are consistent with the data of Buechler et al. (Buechler, C. et al. (1999) “LIPOPOLYSACCHARIDE INHIBITS THE EXPRESSION OF THE SCAVENGER RECEPTOR CLA-L IN HUMAN MONOCYTES AND MACROPHAGES,” Biochem. Biophys. Res. Commun. 262:251-254), who first provided evidence that the expression of CLA-1, the human homologue of SR-BI/CLA-1, is suppressed by high doses of LPS in human monocytes and macrophages. In that study, LPS markedly inhibited CLA-1 mRNA and protein expression at a concentration of 1 μg/ml. In the above-reported experiments, LPS is an inhibitor of SR-BI/CLA-1 and ABCA1 mRNA expression as well as SR-BI/CLA-1 protein expression at 0.2 ng/ml, a concentration that can be pathophysiologically relevant.

SR-BI/CLA-1 and its human homologue CLA-1, both membrane proteins, are highly expressed in the liver, adrenal gland, and ovary (Kalayoglu, M. V. et al. (1998) “A CHLAMYDIA PNEUMONIAE COMPONENT THAT INDUCES MACROPHAGE FOAM CELL FORMATION IS CHLAMYDIAL LIPOPOLYSACCHARIDE,” Infect. Immun. 66:5067-5072) as well as in atherosclerotic lesions of ApoE-deficient mice (Iiyama, K. et al. (1999) “PATTERNS OF VASCULAR CELL ADHESION MOLECULE-1 AND INTERCELLULAR ADHESION MOLECULE-1 EXPRESSION IN RABBIT AND MOUSE ATHEROSCLEROTIC LESIONS AND AT SITES PREDISPOSED To LESION FORMATION,” Circ. Res. 85:199-207). SR-BI/CLA-1 binds HDL with high affinity and mediates uptake of esterified and free cholesterol from HDL in liver and steroidogenic tissues (Acton, S. L. et al. (1996) “IDENTIFICATION OF SCAVENGER RECEPTOR SR-BI AS A HIGH DENSITY LIPOPROTEIN RECEPTOR,” Science 271:518-520; Murao, K. et al. (1997) “CHARACTERIZATION OF CLA-1, A HUMAN HOMOLOGUE OF RODENT SCAVENGER RECEPTOR BI, AS A RECEPTOR FOR HIGH DENSITY LIPOPROTEIN AND APOPTOTIC THYMOCYTES,” J. Biol. Chem. 272:17551-17557). In addition to its role in cholesterol delivery to peripheral tissues, recent experimental data strongly suggest a possible physiological role of SR-BI/CLA-1 in the cellular efflux of both cholesterol from intracellular pools and plasma membrane sterols to HDL. According to recently reported data, SR-BI/CLA-1-mediated bidirectional cholesterol flux is not a result of the tethering of the donor or acceptor particle to the cell surface receptor but is rather due to the lipid organization changes within the plasma membrane lipid bilayer (de la Llera-Moya, M. et al. (1999) “SCAVENGER RECEPTOR BI (SR-BI) MEDIATES FREE CHOLESTEROL FLUX INDEPENDENTLY OF HDL TETHERING TO THE CELL SURFACE,” J. Lipid Res. 40:575-580; Rothblat, G. H. (1999) “CELL CHOLESTEROL EFFLUX: INTEGRATION OF OLD AND NEW OBSERVATIONS PROVIDES NEW INSIGHTS,” J. Lipid Res. 40:781-796). Thus, assuming the ability of SR-BI/CLA-1 to induce changes in the lipid domain organization within the plasma membrane, the mechanism of its participation in the exchange of cholesterol between the cell plasma membranes and phospholipid-containing acceptors can be regarded as gradient diffusion. The direction of the cholesterol movement is considered to be determined by its differential concentration gradient across the membrane as well as by the type of lipoprotein particle and cell type (de la Llera-Moya, M. et al. (1999) “SCAVENGER RECEPTOR BI (SR-BI) MEDIATES FREE CHOLESTEROL FLUX INDEPENDENTLY OF HDL TETHERING To THE CELL SURFACE,” J. Lipid Res. 40:575-580; Rothblat, G. H. (1999) “CELL CHOLESTEROL EFFLUX: INTEGRATION OF OLD AND NEW OBSERVATIONS PROVIDES NEW INSIGHTS,” J. Lipid Res. 40:781-796). Consistent with this concept, it seems reasonable to suggest that in macrophages, SR-BI/CLA-1 is more likely to be involved in the promotion of cholesterol efflux rather than in sterol transfer into the cells. Therefore, functional SR-BI/CLA-1, which is apparently responsible for the passive constituent of HDL-mediated cholesterol efflux, should be of particular importance, since macrophages are the predecessors of foam cells. From this point of view, the present findings, revealing a suppressive effect of LPS on SR-BI/CLA-1 expression in mouse macrophages indicate that the diffusive component of cholesterol efflux, which is likely mediated by SR-BI/CLA-1, might be markedly impaired upon the exposure of cells to endotoxin.

In addition to the LPS inhibitory effect on SR-BI/CLA-1 mRNA and protein expression, the results demonstrate LPS's ability to down regulate ABCA1 expression. This transporter encodes a membrane protein that plays a critical role in ApoA-I-dependent cholesterol and phospholipid efflux from cells (Oram, J. F. (2000) “TANGIER DISEASE AND ABCA1,” Biochim. Biophys. Acta 1529:321-330). ABCA1 has been found to have key functions in regulating HDL plasma concentrations and the balance of cholesterol within the cell (Wade, D. P. et al. (2001) “REGULATION OF THE CHOLESTEROL EFFLUX GENE, ABCA1,” Lancet 357:161-163). The crucial step towards understanding the physiological role of this protein was the identification of ABCA1 as the defective gene in Tangier disease, a rare disorder characterized by very low plasma HDL and the inability of cells to efflux intracellular cholesterol to lipid-poor ApoA-I, the primary HDL precursor (Oram, J. F. (2000) “TANGIER DISEASE AND ABCA1,” Biochim. Biophys. Acta 1529:321-330). Thus, mutations within the ABCA1 gene result in the impairment of the first stage in reverse cholesterol transport, cholesterol transfer from intracellular compartments to the plasma membrane. Ineffective cholesterol efflux permits formation of foam cells, the progenitors of arterial lesions. Immunocytochemical studies have demonstrated that ABCA1 is presumably localized on the plasma membrane (Lawn, R. M. et al. (1999) “THE TANGIER DISEASE GENE PRODUCT ABC 1 CONTROLS THE CELLULAR APOLIPOPROTEIN-MEDIATED LIPID REMOVAL PATHWAY,” J. Clin. Investig. 104:R25-R31; Wang, N. et al. (2000) “SPECIFIC BINDING OF APOA-I, ENHANCED CHOLESTEROL EFFLUX, AND ALTERED PLASMA MEMBRANE MORPHOLOGY IN CELLS EXPRESSING ABC1,” J. Biol. Chem. 275:33053-33058), and according to more recent observations, it is also present in the cytosol and Golgi compartment of unstimulated fibroblasts (Schmitz, G. et al. (2001) “ABC TRANSPORTERS AND CHOLESTEROL METABOLISM,” Front. Biosci. 6:D505-D514) as well as on intracellular vesicles of stably and transiently transfected HeLa cells (Neufeld, E. B. et al. (2001) “CELLULAR LOCALIZATION AND TRAFFICKING OF THE HUMAN ABCA1 TRANSPORTER,” J. Biol. Chem. 276:27584-27590). These last observations provide new insights into its important role in intracellular cholesterol trafficking.

With the known important role of ABCA1 as the mediator of cholesterol efflux, our data demonstrating that extremely low LPS concentrations cause almost complete suppression of ABCA1 expression suggest another intriguing possibility: the combined inhibitory effects of LPS on the expression of the SR-BI/CLA-1 and ABCA1 genes may severely impair both components of the efflux process. This includes gradient diffusion facilitated by SR-BI/CLA-1, as well as the ABCA1-mediated component, including intracellular trafficking of lipids with their subsequent delivery onto the outer lipid bilayer leaflet of the plasma membrane.

Additional experimental evidence of LPS's possible role as a potent proatherogenic stimulus is provided by the data demonstrating its ability to down regulate specific HDL binding as well as HDL-mediated cholesterol efflux. According to the results obtained in our study, LPS dramatically inhibited (up to 20% of control level) specific HDL binding and moderately decreased (50% inhibition of control level) cholesterol efflux to HDL in cultured RAW cells. In the above-reported study, the effective dose of LPS able to elicit 50% inhibition of the HDL-mediated cholesterol efflux turned out to be essentially higher than the dose required for 50% decreases of SR-BI/CLA-1 and ABCA1 mRNA expression. Different experimental conditions could possibly be the cause of the observed differences. Unlike RT-PCR and Western blotting analyses, the cholesterol efflux estimation was performed 24 h after the withdrawal of LPS from cultured cells. As a result, there is a reasonable expectation that within the subsequent 24 h (the duration of the cholesterol efflux experiment) following LPS removal from cells, its suppressive effect upon HDL receptor gene expression can be partially reversed.

Lipopolysaccharides are composed of the O antigen and the core part (Kalayoglu, M. V. et al. (1998) “A CHLAMYDIA PNEUMONIAE COMPONENT THAT INDUCES MACROPHAGE FOAM CELL FORMATION IS CHLAMYDIAL LIPOPOLYSACCHARIDE,” Infect. Immun. 66:5067-5072). The latter includes lipid A, the biologically active portion (Ziihringer, D. et al. (1999) “CHEMICAL STRUCTURE OF LIPID A: RECENT ADVANCES IN STRUCTURAL ANALYSIS OF BIOLOGICALLY ACTIVE MOLECULES,” p. 93-115. In H. Brade, D. C. Morrison, S. Opal, and S. Vogel, (ed.), Endotoxin In Health And Disease. Marcel Dekker, Inc., New York, N.Y.), which also anchors LPS in the outer bacterial wall (Galanos, C. et al. (1985) “SYNTHETIC AND NATURAL ESCHERICHIA COLI FREE LIPID A EXPRESS IDENTICAL ENDOTOXIC ACTIVITIES,” Em. J. Biochem. 148:1-5). It is generally assumed that the lipid A component is responsible for the endotoxic properties of LPS (Galanos, C. et al. (1985) “SYNTHETIC AND NATURAL ESCHERICHIA COLI FREE LIPID A EXPRESS IDENTICAL ENDOTOXIC ACTIVITIES,” Em. J. Biochem. 148:1-5, Takayama, K. et al. (1984) “INFLUENCE OF FINE STRUCTURE OF LIPID A ON LIMULUS AMEBOCYTE LYSATE CLOTTING AND TOXIC ACTIVITIES,” Infect. Immun. 2:350-355). Some experimental evidence indicates that the LPS polysaccharide chain and phosphoryl groups may contribute to the some of the LPS effects on macrophages. In our study we compared various modified LPS preparations in terms of their ability to induce suppression of the ABCA1 and SR-BI/CLA-1 genes, as well as to stimulate IL-1β mRNA expression. It is found that the Re595 mutant LPS, which has been used in all of the above-reported experiments, as well as Re595 DPLA displayed the same activity as the complete form of LPS from E. coli. Re595 MPLA appeared to be a less potent modulator of SR-BI, ABCA1, and IL-1β gene expression. With a higher LPS concentration, the various LPS preparations demonstrated equal inhibitory effects upon the SR-BI/CLA-1 protein expression. These results indicate that the phosphorylated lipid A portion of LPS is required for maximal LPS effects on SR-BI/CLA-1 and ABCA1 gene expression. Apparently, LPS modifications that do not affect the negative charge of its glycolipid interface (lipid A) do not markedly alter these endotoxin activities.

While investigating the LPS-mediated effects on SR-BI/CLA-1 and ABCA1 gene expression in RAW cells, parallel studies of IL-1β mRNA changes have been conducted. The results demonstrate a dose and time dependence of IL-1β mRNA expression similar to that for the negatively regulated LPS-responsive genes. These data suggest the involvement of a similar, if not the same, signaling cascade for the LPS effects on ABCA1, SR-BI/CLA-1, and IL-1β gene expression. Numerous studies have demonstrated that the LPS activation of monocytes and macrophages is associated with NF-KB activation (Guha, M. et al. (2001) “LPS INDUCTION OF GENE EXPRESSION IN HUMAN MONOCYTES,” Cell. Signal. 13:85-94; Manna, S. K. (2000) “WORTMANNIN INHIBITS ACTIVATION OF NUCLEAR TRANSCRIPTION FACTORS NF-KAPPAB AND ACTIVATED PROTEIN-1 INDUCED BY LIPOPOLYSACCHARIDE AND PHORBOL ESTER,” FEBS Lett. 473:1-126; Muller, J. M. et al. (1993) “NUCLEAR FACTOR KAPPA B, A MEDIATOR OF LIPOPOLYSACCHARIDE EFFECTS,” Immunobiology 187:233-256). It has been demonstrated that transcription factor NF-KB is critical for the expression of multiple genes involved in inflammatory responses (Baenerle, P. A. (1997) “NF-KAPPA B AS A FREQUENT TARGET FOR IMMUNOSUPPRESSIVE AND ANTI-INFLAMMATORY MOLECULES,” Adv. Immunol. 65:111-137; Cogswell, J. P. et al. (1994) “NF-KAPPA B REGULATES IL-L TRANSCRIPTION THROUGH A CONSENSUS NF-KAPPA B BINDING SITE AND A NONCONSENSUS CRE-LIKE SITE,” J. Immunol. 153:712-723; Guha, M. et al. (2001) “LPS INDUCTION OF GENE EXPRESSION IN HUMAN MONOCYTES,” Cell. Signal. 13:85-94). The data presented herein evaluating the inhibitory effect of LPS on ABCA1 and SR-BI/CLA-1 gene expression by utilizing the NF-KB inhibitor TPCK clearly demonstrate the involvement of NF-KB activation with the observed LPS effect. NF-KB may interact directly with the promoter binding sequences blocking SR-BI/CLA-1 and ABCA1 gene transcription, an effect never reported for NF-KB. Alternatively, NF-KB may interact indirectly, through an unknown intermediate messenger(s) that is able to down regulate gene transcription. According to the recent data of Panousis and Zuckerman (Panousis, C. G. et al. (2000) “INTERFERON-GAMMA INDUCES DOWNREGULATION OF TANGIER DISEASE GENE (ATP-BINDING-CASSETTE TRANSPORTER 1) IN MACROPHAGE-DERIVED FOAM CELLS,” Arterioscler. Thromb. Vasc. Biol. 20: 1565-1571), the proinflammatory lymphokine gamma interferon is capable to induce down regulation of ABCA1 gene expression in macrophage-derived foam cells. Another recent report provides experimental evidence that gamma interferon is able to modulate intracellular signaling responses of the LPS-initiated NF-KB pathway. It has been demonstrated that priming macrophages with gamma interferon strongly enhanced IKB-a degradation in response to LPS, resulting in a significant increase in NF-KB DNA binding activity (Held, T. K. et al. (1999) “GAMMA INTERFERON AUGMENTS MACROPHAGE ACTIVATION BY LIPOPOLYSACCHARIDE BY TWO DISTINCT MECHANISMS, AT THE SIGNAL TRANSDUCTION LEVEL AND VIA AN AUTOCRINE MECHANISM INVOLVING TUMOR NECROSIS FACTOR ALPHA AND INTERLEUKIN-1,” Infect. Immun. 67:206-212). Alternatively, the pathway of the observed LPS suppressive effect on HDL receptor gene expression could possibly involve an apoptotic mechanism, since LPS itself as well as LPS-induced cytokines, including TNF-a, IL-1β, and gamma interferon, are known to be potent apoptotic factors (15, 57). In the experiments reported herein, no morphological changes typical for apoptosis or any apparent evidence of cytotoxicity are observed.

In conclusion, the invention provides new insights into the possible role of LPS. Previous studies have shown LPS to be proatherogenic, able to induce chronic inflammation and subsequent foam cell formation, which is the hallmark of early lesions in atherosclerosis (Ross, R. (1993) “THE PATHOGENESIS OF ATHEROSCLEROSIS: A PERSPECTIVE FOR THE 1990s,” Nature 362:801-809). The data demonstrating that LPS down regulated the gene expression of two key HDL binding proteins involved in cholesterol efflux provide a potential mechanism for LPS contributing to atherogenesis: the serious impairment of pathways that are primarily responsible for HDL production and normal excretion of cholesterol. The results clearly suggest that the mechanism of the LPS inhibitory effect on both genes is NF-KB dependent but may involve an unknown intermediate factor(s), which remains to be identified. Considering the critical roles of SR-BI/CLA-1 and the ABCA1 transporter in the regulation of HDL metabolism and cholesterol homeostasis, further investigation of the mechanisms underlying the suppressive effect of LPS is important for a better understanding of HDL metabolism and atherogenesis.

### Example 2

### Binding and Internalization of Lipopolysaccharide by CLA-1, A Human Orthologue of Rodent Scavenger Receptor B1

### Materials and Methods

Lipopolysaccharides, E. Coli B4:0111, Salmonella minnesota Re 595, Diphosphoryllipid A (DPLA) and Monophosphoryl lipid A (MPLA) are purchased from Sigma. Lipopolysaccharides from E. coli K12 strain LCD25 (unlabeled and 3H-metabolically labeled) were purchased from List Biological Laboratories. Rabbit anti-SR-BI/CLA-1 antibody cross-reacting with the human homologue CLA-1, is from Novus Biological.

Raw Cells. Mouse monocyte-macrophages, RAW 264.7 (ATCC [American Type Culture Collection] TIB 71), are grown in 12-well plates in Dulbecco modified Eagle medium (DMEM), supplemented with 10% fetal bovine serum (FBS), penicillin (100 U/ml), and streptomycin (100 μg/ml) in a humidified atmosphere containing 5% CO2 and 95% air at 37° C.

CLA-1 Overexpression HeLa cells. HeLa (Tet-off) cells (Clontech, Pal Alto, Calif.) are grown in DMEM (Invitrogen), supplemented with 10% fetal calf serum, 2 mM glutamine, 100 IU/ml of penicillin, 100 μg/ml of streptomycin, and 100 μg/ml of G418. Cells were transfected with FuGENE-6 (Roche Diagnostics), using the expression plasmid pTRE2 (Clontech, Pal Alto, Calif.), encoding a CLA-1 protein (pTRE2-CLA-1). Cells were co-transfected with pTRE2-CLA-1 and pTK-Hyg (Clontech, Pal Alto, Calif.), using a 1:20 ratio, and selected with 400 μg/ml of hygromycin. Hygromycin resistant cells were screened for the expression of the CLA-1 protein by utilizing rabbit anti-SR-BI/CLA-1 (Novus Biological, Inc) by Western blotting.

HDL, Apolipoprotein Isolation And Labeling. Human HDL₂₊₃ (1.072<d<1.216) is isolated from the plasma of healthy donors by two repetitive centrifugations by the method of Redgrave, T. G. et al. (1975) (“SEPARATION OF PLASMA LIPOPROTEINS BY DENSITY-GRADIENT ULTRACENTRIFUGATION,” Anal. Biochem. 65:4249). The HDL₂₊₃ is passed through an agarose-heparin column (HiTrap, Amersham Pharmacia Biotech), and an apoE-free free HDL fraction is collected. Apolipoproteins are purified from human plasma (Remaley, A. T. (2001) “APOLIPOPROTEIN SPECIFICITY FOR LIPID EFFLUX BY THE HUMAN ABCA1 TRANSPORTER,” Biochem. BiophysRes. Commun. 280:818-823), and are over 99% pure, as determined by SDS-PAGE and amino-terminal sequence analysis. Labeling of HDL, apoA-I and apoA-Ii with 125I-Na is performed by the N-bromosuccinimide method according to Sinn, H. J. et al. (1988) (“RADIOIODINATION OF PROTEINS AND LIPOPROTEINS USING N-BROMOSUCCINIMIDE AS OXIDIZING AGENT,” Anal. Biochem. 170, 186-192). The specific radioactivities range from 1000 to 3000 cpm/ng of protein with more than 98% of the radioactivity being protein associated. Human HDL labeled with [³H] cholesteryl oleoyl ether (CE), a nonhydrolyzable cholesteryl ester analogue was prepared by a modification of the procedure of Miyazaki, A. et al. (1994) (“ACETYLATED LOW DENSITY LIPOPROTEIN REDUCES ITS LIGAND ACTIVITY FOR THE SCAVENGER RECEPTOR AFTER INTERACTION WITH RECONSTITUTED HIGH DENSITY LIPOPROTEIN,” J. Biol. Chem. 269:5264-5269). FPLC analysis demonstrate more than 95% of [³H]CE associated with the fraction corresponding to native HDL. The specific radioactivity for HDL-[³H]CE was 12-20 dpm/ng of HDL protein.

Western Blot Analysis. Western blot analysis is performed, as described by Bocharov, A. V. et al. (2001) (“CHARACTERIZATION OF A 95 KDA HIGH AFFINITY HUMAN HIGH DENSITY LIPOPROTEIN-BINDING PROTEIN,” Biochemistry 40:4407-4416). Cell proteins are extracted with 2% Triton X-100 in TBS, pH 7.4. The extracts are precipitated by adding methanol to a final concentration of 90%. Precipitated proteins are dissolved in 2×SDS-PAGE sample buffer and applied on a 7.5% SDS-PAGE under reducing conditions. Anti-SR-BI/CLA-1 antibody at a dilution of 1:1000 is used as the first antibody, and a sheep anti-rabbit IgG antibody conjugated with alkaline phosphatase (Sigma) is used as the second antibody. For protein normalization, mouse anti-human β-actin antibody at a dilution of 1:2500 is used as the first antibody, and a sheep anti-mouse IgG antibody conjugated with alkaline phosphatase (Sigma) is used as the second antibody.

HDL-Binding And Cholesteryl Oleoyl Uptake Assays. Saturation binding experiments are performed at 4° C. using ¹²⁵I-HDL concentrations between 1.25 and 40 μg/ml. The cells are incubated with ice-cold Hanks Balanced Salt Solution (HBSS) containing 20 mg/ml of BSA (HBSS/BSA) and labeled ligand in the presence or absence of 20-fold excess unlabeled HDL. After a 2 hr-incubation on ice, specific binding is determined as reported by Bocharov, A. V. et al. (2001) (“CHARACTERIZATION OF A 95 KDA HIGH AFFINITY HUMAN HIGH DENSITY LIPOPROTEIN-BINDING PROTEIN,” Biochemistry 40:4407-4416).

HDL-[³H]CE uptake experiments are performed in a serum free DMEM containing 0.2% BSA. Cell monolayers are incubated with various concentrations of HDL-[3H]CE in the presence (nonspecific uptake) or absence (total uptake) of 25-fold excess of the unlabeled HDL for 20 hr. Specific uptake is determined as the difference between total and nonspecific uptake.

LPS-Binding Assay. The lipopolysaccharide 0111:B4 (Sigma) is iodinated as reported by Ulevitch, R J. (1978) (“THE PREPARATION AND CHARACTERIZATION OF A RADIO IODINATED BACTERIAL LIPOPOLYSACCHARIDE,” Immunochemistry 15:157-164). Saturation binding experiments are performed at 4° C. using ¹²⁵I-LPS concentrations between 1.25 and 40 μg/ml. All incubations are performed in Hank's balanced salt solution (HBSS) containing 20 mg/ml BSA which monomerized and formed complex with LPS. Non-specific binding is determined in the presence of 50-fold excess unlabeled LPS. After a 2-hr-incubation on ice, the cells are rinsed with ice-cold HBSS and utilized for radioactivity measurements as reported by Bocharov, A. V. et al. (2001) (“CHARACTERIZATION OF A 95 KDA HIGH AFFINITY HUMAN HIGH DENSITY LIPOPROTEIN-BINDING PROTEIN,” Biochemistry 40:4407-4416). Specific binding is determined as the difference between total and non-specific binding, and normalized by protein content.

Competition Binding Experiments. RAW cells are cultured for 24 hr in serum free DMEM before experiment. After chilling on ice, cells are incubated in the presence of 5 μg/ml of ¹²⁵I-HDL, 1 μg/ml ¹²⁵I-apoA-I, 1 μg/ml ¹²⁵I-apoA-II, and increasing concentrations of cold ligands (HDL, apoA-I, apoA-II and E. coli 0111:B4 LPS) for 1 hour in HBSS/BSA. Cell radioactivity is measured as described in the “LPS-binding assay” section.

LPS-Uptake And Internalization Assays. For measurement of LPS-uptake and internalization, cells are incubated in a CO₂ incubator for different time periods in DMEM, 20 mg/ml BSA containing 1 μg/ml of ¹²⁵I-LPS in the presence or absence of 200× excess of unlabeled ligand. At specified time points, cells were chilled on ice and rinsed for three times with ice-cold PBS followed by a 20-min treatment with 0.05% trypsin, 5 mM EDTA, 150 mM NaCl solution on ice. Trypsin-released radioactivity was determined as surface-bound ligand. Cell-associated radioactivity counted after hydrolysis in 1 N NaOH is considered as internalized LPS. Specific binding and internalization are determined as the difference between total and non-specific binding/internalization (the amount of radioactivity measured in the presence of 200× fold excess unlabelled ligand).

Preparation of BODIPY-LPS and Alexa 568 HDL, Lipid-Free ApoA-1 And ApoA-II. HDL, apoA-I and apoA-II are conjugated with Alexa-568/488, SE (Molecular Probes, protein labeling kit) following the kit instructions. The Alexa ligands are analyzed by 10-20% Tricine-SDS peptide gel electrophoresis. Gels are scanned using a Fluorocsan (model A, Hitachi). Alexa-labeled preparations of HDL and apolipoproteins are found in appropriate positions with molecular masses of 28 and 18 kDa for apoA-1 and apoA-II respectively. Re-LPS was labeled using the BODIPY*FL, SE labeling kit from Molecular Probes, Inc. (Eugene, Oreg.) following the manufacturer's suggested procedure and modifications reported by Levels, J. H. et al. (2001) (“DISTRIBUTION AND KINETICS OF LIPOPROTEIN-BOUND ENDOTOXIN,” Infect. Immun. 69, 2821-2828).

Uptake of BODIPY-LPS and Alexa 568 HDL; ApoA-I/LPS Co-Localization Experiments. HeLa cells cultured on collagen-coated glass micro-slides are incubated with 5 μg/ml of Alexa 568 HDL, 1-0.5 μg/ml of Alexa 568 apoA-I, 1-0.5 μg/ml of Alexa 568 apoA-II or 0.5 μg/ml Bodipy-LPS for 1-2 hour in a CO₂ incubator in DMEM containing 20 mg/ml BSA. For quenching experiments, BODIPY-LPS (1 μg/ml) is incubated with cells for 30 min followed by being washed with ice-cold PBS and fixed with 4% paraformaldehyde. Imaging is performed in PBS containing 0.4 mg/ml of Trypan Blue as the quenching agent. The effect of apoA-I on LPS uptake is studied by incubating HeLa cells with 0.5 μg/ml Bodipy-LPS in the presence of 100 μg/ml of lipid poor apoA-1 for 1-2 hours in a CO₂ incubator. For co-localization, Bodipy-LPS and Alexa-568-apoA-I are used at the same concentration of 0.5 μg/ml. A Nikon video-imaging system, consisting of a phase contrast inverted microscope equipped with set of objectives and filters for immunofluorescence and connected to a digital camera and image processor, is used for recording Alexa-568-HDL and Bodipy-LPS uptake. For co-localization experiments, fluorescence is viewed with a Zeiss 510 laser scanning confocal microscope, using a krypton-argon-Omnichrome laser with excitation wavelengths of 488 and 568 nm for Bodipy-LPS and Alexa-568, respectively.

Preparation of Bodipy-LPS/Alexa488-Apolipoprotein Labeled HDL Complexes. Alexa488-apolipoprotein labeled HDL (5 mg) are mixed with Bodipy-LPS (5 μg) in a final volume of 1 ml followed by the addition of 2 ml delipidated human plasma and incubated for 24 hours at 37° C. Bodipy-LPS/Alexa488-apolipoprotein labeled HDL complexes are re-isolated by a centrifugation in a NaBr gradient (1.072<d<1.216). After extensive dialysis against Ca²⁺, Mg²⁺ free PBS, the complexes are filtered (0.22 μm) and stored in a refrigerator up to 2 weeks. The purity of the complexes as determined by fluorescent scanning of native PAGE and agarose gel electrophoresis is close to 100%.

Uptake of Bodipy-LPS/Alexa488-Protein Labeled HDL Complexes. The surface binding of the LPS/HDL complex is studied by incubating of 10 μg/ml of doubly labeled HDL (Bodipy-LPS and Alexa 488-HDL) for 2 h with CLA-1 overexpressing or mock transfected HeLa cells at 4° C. and examined by confocal microscopy. Internalization of the complex is analyzed after three washings with ice-cold Ca²⁺, Mg²⁺ free PBS followed by incubation at 37° C. for 4-hour period in fresh serum free culture medium. A separate sample of HDL (10 μg/ml) is incubated with HeLa cells at 37° C. for the 1- and 4-hour periods.

Preparation of 3H-LPS/HDL and LPSp25I-HDL Complexes. HDL (5 mg) were mixed with ³H-metabolically labeled LPS (150 μg, LCD25) in a final volume of 1 ml, followed by the addition of 2 ml of delipidated human plasma and incubation for 24 h at 37° C. 30 μg of non-labeled LPS (LCD25) is incubated with 1 mg of ¹²⁵I-HDL in a final volume of 200 μl, followed by the addition of 0.4 ml of delipidated human plasma and incubation for 24 h at 37° C. Both ³H-LPS/HDL and LPS/¹²⁵I-HDL complexes are re-isolated and analyzed as described above for BodipyLPS' Alexa HDL complexes. The specific radioactivity for HDL-3H-LPS was 12-14 dpmlng of HDL protein.

Selective LPS Uptake. The selective LPS uptake is examined by incubating 10 μg/ml LPS-labeled HDL ³H-metabolically labeled LPS) for 2 h with CLA-1 overexpressing or mock transfected HeLa cells at 37° C. in the presence or absence of a 100-fold excess of cold HDL. In a parallel experiment, the cells were incubated with 10 μg/ml LPS/¹²⁵I HDL in the presence of 100-fold excess of unlabeled HDL. Specific HDL uptake was determined as previously reported (Ulevitch, R J. (1978) “THE PREPARATION AND CHARACTERIZATION OF A RADIOIODINATED BACTERIAL LIPOPOLYSACCHARIDE,” Immunochemistry 15:157-164). ¹²⁵I-Apolipoprotein and ³H-LPS-lipoprotein uptake is expressed in terms of apparent particle uptake. Based on the specific activity of the labeled lipoprotein preparations, the amount of lipoprotein that would be necessary to deliver the observed amount of tracer was calculated.

Degradation of HDL. Degradation of HDL was determined, using the following previously reported pulse-chase scheme (Silver, D. L. et al. (2001) “HIGH DENSITY LIPOPROTEIN (HDL) PARTICLE UPTAKE MEDIATED BY SCAVENGER RECEPTOR CLASS B TYPE 1 RESULTS IN SELECTIVE SORTING OF HDL CHOLESTEROL FROM PROTEIN AND POLARIZED CHOLESTEROL SECRETION,” J. Biol. Chem. 276:25287-25293). Briefly, cultured CLA-1 overexpressing and mock transfected HeLa cells were pulsed for 1 h at 37° C. with radiolabeled ¹²⁵I-HDL/LPS complexes. Cells were then cooled on ice and washed 3 times with binding buffer. Following the washes, cells were returned to 37° C. and chased for 2 h in binding buffer in the absence of radiolabeled lipoproteins. At the completion of the chase period, cell media was collected and trichloroacetic acid-precipitable counts were determined as a measurement of degradation (Silver, D. L. et al. (2001) “HIGH DENSITY LIPOPROTEIN (HDL) PARTICLE UPTAKE MEDIATED BY SCAVENGER RECEPTOR CLASS B TYPE 1 RESULTS IN SELECTIVE SORTING OF HDL CHOLESTEROL FROM PROTEIN AND POLARIZED CHOLESTEROL SECRETION,” J. Biol. Chem. 276:25287-25293). Cells were also lysed, and radioactivity and protein concentration was measured.

Sites Of LPS Delivery. For studying the sites of LPS delivery, cells are incubated with 1 μg/ml Bodipy-LPS at 37° C. for 2 hours, then washed and chased at 37° C. for 30 minutes in the presence of Bodipy-transferrin or Bodipy-ceramide BSA complex. In separate experiments, instead of BSA-monomerized Bodipy-LPS, the cells are incubated with 10 μg/ml of HDL bound Bodipy-LPS to determine the sites of LPS transport when associated with HDL. In separate experiments, instead of BSA-monomerized Bodipy-LPS, the cells were incubated with 10 μg/ml HDL-bound Bodipy-LPS to determine the sites of LPS transport when associated with HDL.

### Results

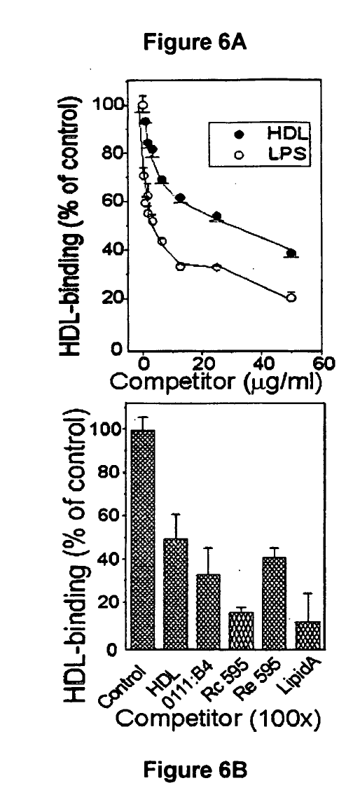

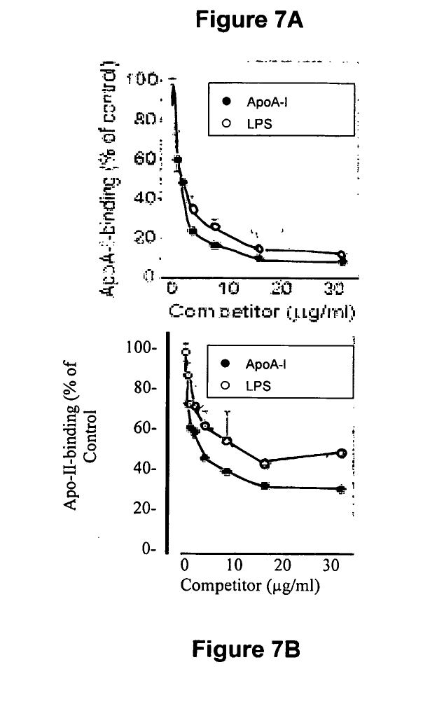

Competition of LPS with SR-BI/CLA-1 Ligands. The competition of LPS with HDL, which is known to bind to SR-BI/CLA-1, may be analyzed in RAW cells, which have a high level of SR-BI/CLA-1 expression (Baranova, I. et al. (2002) “LIPOPOLYSACCHARIDE DOWN REGULATES BOTH SCAVENGER RECEPTOR B1 AND ATP BINDING CASSETTE TRANSPORTER A1 IN RAW CELLS,” Infect. Immun. 70:2995-3003). As seen in FIGS. 6A and 6B, LPS (E. Coli B4:0111) competed with iodinated HDL in a dose-dependent manner (FIG. 6A). Various forms of LPS are also evaluated, including 0111:B4, Re595, Rc595 and DPLA, all of which similarly decreased ¹²⁵I-HDL-binding (FIG. 6B) in RAW cells. Because exchangeable HDL apolipoproteins are considered as the primary ligands for HDL-binding, a competition of LPS with isolated lipid poor apolipoproteins was studied. LPS efficiently competes with ¹²⁵I-apoA-1 and ¹²⁵I-apoA-II (FIGS. 7A and 7B). Nearly similar competition curves are observed when unlabeled apolipoproteins are used as competitors. Because the experiments are conducted on ice, which prevents the formation of a complex between LPS and lipoproteins (Netea, M. G. et al. (1998) “BACTERIAL LIPOPOLYSACCHARIDE BINDS AND STIMULATES CYTOKINE-PRODUCING CELLS BEFORE NEUTRALIZATION BY ENDOGENOUS LIPOPROTEINS CAN OCCUR,” Cytokine 10:766-772), the HDL, apolipoproteins and LPS interacted with HDL-receptor as independent ligands.

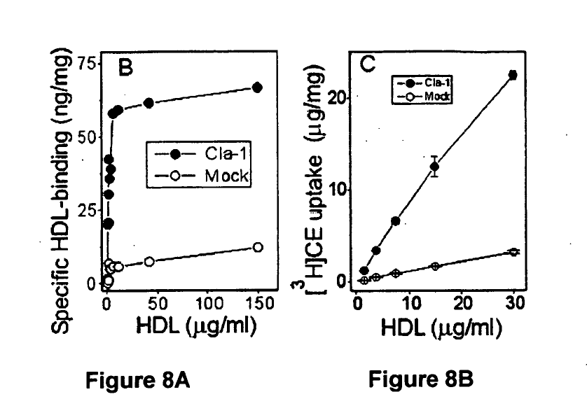

CLA-1 Expression, HDL-Binding And Cholesterol Ester Uptake In Stably Transfected Hela Cells. In light of the present findings that LPS is a potent competitor for SR-BI/CLA-1 related ligands, the ability of SR-BI/CLA-1 to function in LPS uptake through LPS-binding and internalization is evaluated. Accordingly, human HeLa cells are stably transfected with a vector containing the human SR-BI/CLA-1 receptor, CLA-1. Western blot analyses of stably transfected HeLa cell extracts, using an anti-rodent SR-BI/CLA-1 antibody that cross reacts with CLA-1, reveal a single band with an estimated molecular weight of 83 kDa. An approximately 10-times higher CLA-1 level is observed in CLA-1 overexpressing cells when compared to mock-transfected HeLa cells. A two-day incubation of CLA-1 overexpressing cells with 1 μg/ml of tetracycline diminished the CLA-1 level close to that seen with mock-transfected cells. As seen in FIGS. 8A and 8B, overexpressing cells have increased levels of ¹²⁵I-HDL binding and HDL-CE uptake by more than 10-fold, observations demonstrating the HDL-receptor activity of CLA-1. Scatchard analysis reveals a 10-fold increase of capacity for the H DL-binding site after transfection with a Kd=1-2 μg/ml.

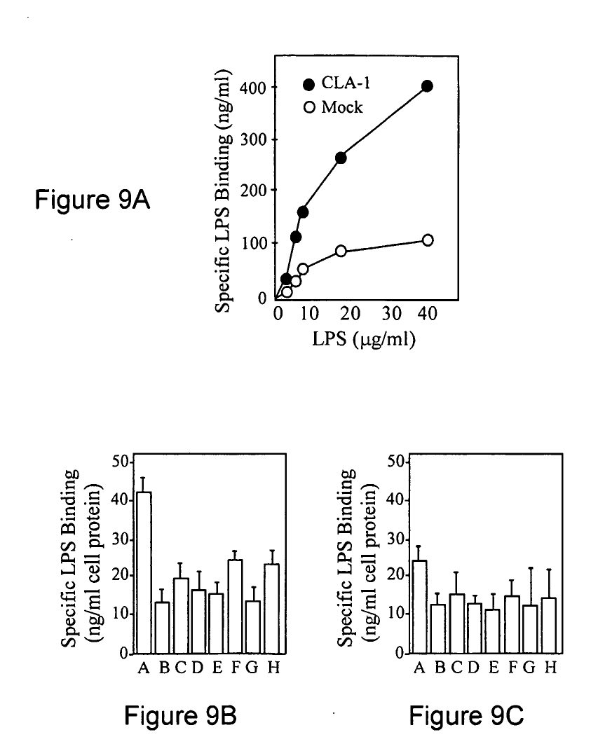

Specific binding of LPS to CLA-1. To examine the possible role of CLA-1 in LPS-binding, ligand-binding analyses are conducted, using iodinated LPS (0111:84). The CLA-1-overexpressing cells demonstrate a 4-5 fold-increase of specific LPS-binding (FIG. 9A). Scatchard analyses demonstrated a high affinity binding site with Kd=16 μg/ml and capacity of 150 ng/mg cell protein in the CLA-1 overexpressing cells. Despite the demonstration of saturatable specific LPS-binding in mock-transfected cells, Scatchard analyses resulted in an indeterminable Kd because of low and variable amount of specific binding.

The ability of O-antigen containing LPS (0111:84) and O-antigen-lacking LPS: Re S9S, DPLA, and MPLA to compete against 125I-LPS (0111:84) was analyzed in CLA-1 overexpressing and mock-transfected cells. As seen at FIG. 9B and FIG. 9C, all unlabelled ligands compete against iodinated LPS in both cell lines. The increased LPS-binding observed in CLA-1 overexpressing cells is effectively competed by the presence of 100× excess unlabeled ligand to a similar level observed in mock-transfected cells. The exchangeable HDL apolipoproteins also effectively compete against LPS-binding. Because essentially all lipid-A variants competed for LPS binding, this indicates that the acetylated di-galactoseamine interface is required for LPS binding rather than O-antigen.

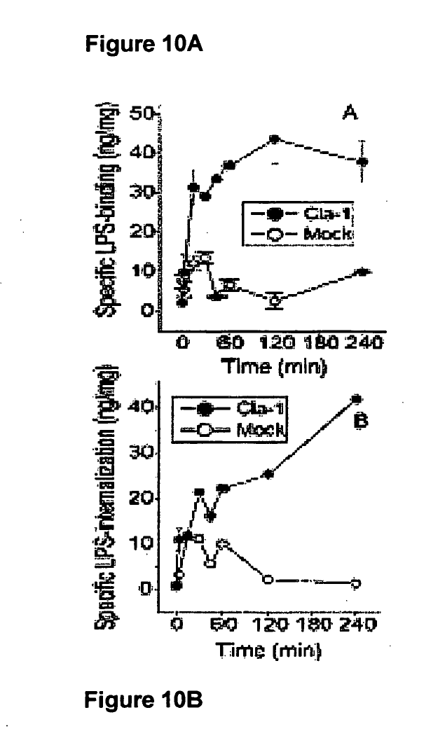

Uptake and internalization of iodinated LPS. As seen in FIG. 10A, CLA-1 overexpressing HeLa cells demonstrate a substantial time-dependent increase in LPS uptake when analyzed at 37° C. Specific binding and internalization of LPS were also increased when analyzed using trypsin treatment to discriminate between surface-bound (FIG. 10A) and internalized LPS (FIG. 10B). Similar results were observed upon FACS-scan analyses of Bodipy-labeled LPS uptake.

Uptake Of Alexa-HDL And Bodipy-LPS In CLA-1 Overexpressing HeLa Cells. CLA-1 overexpressing HeLa cells demonstrate intensive membrane and intracellular staining upon the incubation with Alexa568-HDL. Rare, very faint staining can be observed in some experiments when incubating with mock-transfected cells. CLA-1 overexpression in HeLa cells increases Bodipy-LPS uptake when compared with a mocktransfected control. CLA-1 overexpression induces rapid Bodipy-LPS internalization and delivery into peri-nuclear cellular compartments, as determined in Trypan blue quenching experiments comparing mock transfected and overexpressing cells.

To determine if both apoA-I and LPS are delivered to intracellular compartments via the same pathway, CLA-1 overexpressing HeLa cells are incubated with Bodipy-LPS in the presence of 200× excess of unlabeled lipid poor apoA-I. The presence of high apoA-1 excess dramatically reduces Bodipy-LPS uptake and affects its distribution through intracellular compartments when compared to the absence of apoA-I. Smaller stained vesicles were eventually seen in the cytoplasm with significantly reduced staining in the peri-nuclear area. When incubating CLA-1 overexpressing HeLa cells with equal concentrations of 0.5 μg/ml Alexa568-apoA-I and Bodipy-LPS, a strong area of co-localization (yellow) is demonstrated on the cell surface as well as intracellularly. A similar co-localization of apoA-I and LPS is observed in the RAW cell model, indicating that both the LPS and apoA-1 peri-nuclear transportation is not an artifact of high CLA-1 expression or the result of the use of a particular cell model. Other CLA-1 ligands, such as HDL and apoA-II could be also extensively co-localized with LPS in CLA-1 overexpressing cells.

Sites of Delivery of BSA-monomerized LPS. Co-localization experiments demonstrate that the majority of BSA-monomerized Bodipy-LPS enters the Golgi complex after rapid endocytosis. Intensive co-localization of Bodipy-LPS with ceramide indicates that the Golgi complex rather than endocytic recycling compartment is the primary site of LPS transport by CLA-1. However, a weaker yellow signal could be also detected when Bodipy-LPS loaded cells are chased at 37° C. for 30 min in the presence of Bodipy-transferrin suggesting that some LPS is transported to the endocytic recycling compartment by CLA-1.

Uptake of HDL-Associated LPS in CLA-1 Overexpressing Cells. It has been demonstrated that LPS, an amphipathic molecule which forms micelles in aqueous buffers, is rapidly monomerized by, and forms complexes, with plasma proteins in the plasma. In addition to serum albumin, another important plasma LPS binding protein is HDL, the major ligand for CLA-1. Because HDL has been also demonstrated to neutralize LPS in both in vitro and in vivo experiments, Bodipy-LPS binding and internalization are studied while in a complex with Alexa488 apolipoprotein labeled HDL in CLA-1 overexpressing HeLa cells. This approach allows studying both holoparticle transport and intracellular sorting by directly observing the fluorescent signal from HDL Alexa488-apolipoproteins and Bodipy-LPS. The LPS/HDL complex is found to bind to the plasma membrane after a 2-hour incubation at 4° C. as a holoparticle, since Bodipy-LPS (red) and Alexa 488-HDL (green) merge at the cell surface as a bright yellow staining. No substantial HDL/LPS binding is detected when incubated with mock-transfected cells. After washing unbound ligand and incubating the cells at 37° C. for 4 hrs, very little co-localization can be detected on the cell surface. The mostly green surface staining indicates the presence of HDL associated with the plasma membrane. Intracellularly, holoparticle internalization (yellow) is detected as co-trafficking of labeled components within HDL/LPS as well as a sorting of HDL and LPS to different intracellular compartments (red and green spotting). To determine whether the same sorting process occurs in the continuous presence of an HDULPS complex at 37° C., cells are incubated for 1- and 4-hour periods. An HDL/LPS complex resides initially on the plasma membrane and is rapidly internalized in CLA-1 overexpressing HeLa cells (yellow). Little sorting can be seen after a 1-hour incubation. By 4-hours of HDL/LPS binding, there is surface and intracellular co-localization (yellow), as well as a sorting of HDL and LPS to different intracellular compartments. These data indicate that the metabolism of LPS associated with HDL closely resembles HDL endocytosis and selective apolipoprotein-cholesterol ester sorting by mouse SR-BI/CLA-1 that has been recently reported (Silver, D. L. et al. (2001) “HIGH DENSITY LIPOPROTEIN (HDL) PARTICLE UPTAKE MEDIATED BY SCAVENGER RECEPTOR CLASS B TYPE 1 RESULTS IN SELECTIVE SORTING OF HDL CHOLESTEROL FROM PROTEIN AND POLARIZED CHOLESTEROL SECRETION,” J. Biol. Chem. 276:25287-25293) detailing mechanisms of selective cholesterol ester uptake and sorting to the transferrin recycling compartment in polarized epithelial cells.

Sites Of LPS Delivery Upon HDL/LPS Uptake And Sorting. When Bodipy-LPS is introduced as a complex with HDL, the majority of the LPS/HDL complex is rapidly internalized as a holoparticle (red). Transferrin, a known recycling protein, is found to colocalize with Bodipy-LPS in the perinuclear compartment. The intensity of yellow patches indicates that the majority of HDL-associated Bodipy-LPS reaches the transferrin/endocytic recycling compartment. In contrast to the BSA monomerized LPS, HDL-bound Bodipy-LPS is colocalized with the Bodipy-ceramide BSA complex to a lesser extent, indicating that LPS is predominantly transported to the endocytic recycling compartment instead of to the Golgi network. No signal corresponding to Bodipy-LPS is observed in mock-transfected cells.

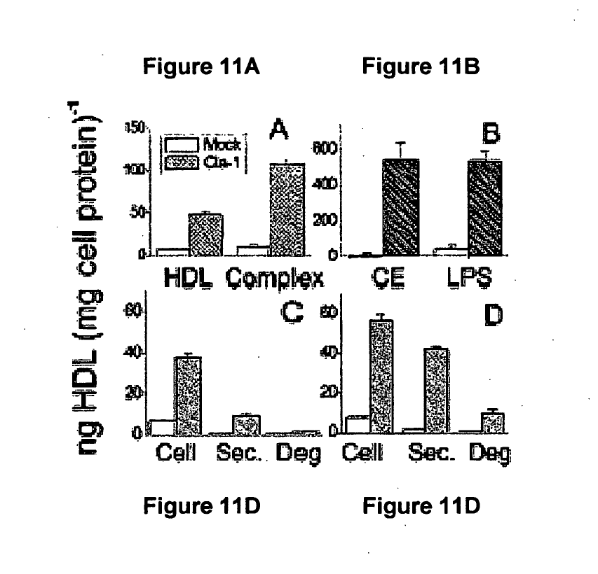

Selective LPS Uptake. To demonstrate that the expression of functional CLA-1 may mediate selective LPS uptake, the uptake of ³H-LPS-labeled HDL and ¹²⁵I HDL/LPS complexes is measured. CLA-1 overexpressing cells demonstrate a markedly increased uptake of both ¹²⁵I HDL and ¹²⁵I HDL/LPS complex when compared with control cells (FIGS. 11A, 11B, 11C, and 11D). Interestingly, HDL apolipoprotein uptake is increased by 2-fold when LPS was associated with HDL, indicating that the LPS motif responsible for CLA-1 binding remains exposed while LPS associated with HDL. In both cells, CLA-1 stably transfected HeLa and mock transfected HeLa cells, 3H-LPS/HDL uptake strongly exceeds apolipoprotein uptake (up to 4-5 fold). Selective LPS uptake was as increased as CE-cholesterol uptake, which also exceeded apolipoprotein uptake by 5-fold. In separate experiments, the effect of CLA-1 overexpression on HDL-retroendocytosis and apolipoprotein degradation is investigated. As can be seen in, 11B, 11C, and 11D, relatively small amounts of HDL apolipoprotein secreted from the cells (10.45 ng/mg/2 h) degraded (1.25 ng/mg/2 h) in CLA-1 overexpressing cells. The presence of LPS in the HDL particle increases HDL uptake by 2-fold during pulse HDL incubations (FIG. 11D). The amounts of chased (secreted) HDL apolipoprotein increased by 4-fold when LPS is present in the complex. The portion of degraded HDL apolipoprotein increases disproportionately, reaching 20% of total secreted HDL apolipoprotein radioactivity.

### Conclusions

SR-BI/CLA-1 is a high affinity HDL/LDL binding protein, which mediates the selective uptake of HDL cholesteryl ester into liver and steroidogenic tissues (Trigatti, B. L. et al. (2000) “CELLULAR AND PHYSIOLOGICAL ROLES OF SR-BI, A LIPOPROTEIN RECEPTOR WHICH MEDIATES SELECTIVE LIPID UPTAKE,” Biochim. Biophys. Acta 1529:276-286); Krieger, M. (2001) “SCAVENGER RECEPTOR CLASS B TYPE I IS A MULTILIGAND HDL RECEPTOR THAT INFLUENCES DIVERSE PHYSIOLOGIC SYSTEMS,” J. Clin. Invest 108:793-797, for review). Highly expressing SR-BI/CLA-1 organs include liver, adrenals, ovary and testis. In steroid-producing tissues, such as the adrenal, most of the cholesterol for steroid hormone production is delivered through SR-BI/CLA-1. In the liver, SRBI is involved with bile formation and appears to be the major mechanism of cholesterol excretion.

Accumulating evidence suggest that the function of SR-BI/CLA-1 is not solely linked to cholesterol ester trafficking, but rather involves a wide spectrum of activities. SR-BI/CLA-1 has been recently demonstrated to be involved in the uptake of apoptotic cells (Imachi, H. et al. (2000) “HUMAN SCAVENGER RECEPTOR B1 IS INVOLVED IN RECOGNITION OF APOPTOTIC THYMOCYTES BY THYMIC NURSE CELLS,” Lab Invest 80:263-270), triglyceride and phospholipid delivery to certain cell types (Urban, S. et al. (2000) “SCAVENGER RECEPTOR BI TRANSFERS MAJOR LIPOPROTEIN-ASSOCIATED PHOSPHOLIPIDS INTO THE CELLS,” J. Biol. Chem. 275:33409-33415), as well as the selective uptake of HDL associated vitamin E (Goti, D. et al. (2001) “SCAVENGER RECEPTOR CLASS B, TYPE I IS EXPRESSED IN PORCINE BRAIN CAPILLARY ENDOTHELIAL CELLS AND CONTRIBUTES TO SELECTIVE UPTAKE OF HDL-ASSOCIATED VITAMIN E,” J. Neurochem. 76:498-508). In the bloodstream, lipoprotein particles function as vesicles carrying hydrophobic core molecules within a phospholipid/cholesterol microenvironment, while surface apolipoproteins function as a ligand for SR-BI/CLA-1 binding. Both of the exchangeable HDL apolipoproteins apoA-1 and apoA-1i have been demonstrated as SR-BI/CLA-1 ligands when in a lipid-poor form (Xu, S. et al. (1997) “APOLIPOPROTEINS OF HDL CAN DIRECTLY MEDIATE BINDING TO THE SCAVENGER RECEPTOR SR-BI, AN HDL RECEPTOR THAT MEDIATES SELECTIVE LIPID UPTAKE,” J. Lipid Res. 38:1289-1298). In addition to apolipoproteins, aminophospholipids containing a negative charge, such as phosphotidyl serines and phosphotidyl ethanolamine, have been demonstrated to be efficient competitors of HDL-binding in SR-BI/CLA-1 overexpressing cells (Imachi, H. et al. (2000) “HUMAN SCAVENGER RECEPTOR B1 IS INVOLVED IN RECOGNITION OF APOPTOTIC THYMOCYTES BY THYMIC NURSE CELLS,” Lab Invest 80:263-270; Urban, S. et al. (2000) “SCAVENGER RECEPTOR BI TRANSFERS MAJOR LIPOPROTEIN-ASSOCIATED PHOSPHOLIPIDS INTO THE CELLS,” J. Biol. Chem. 275:33409-33415).

LPS from gram-negative bacteria are very diverse structures. However, the conserved diphosphorylated glucosamine-based phospholipid known as lipid A carries the endotoxic activity of these molecules (Galanos, C. et al. (1985) “SYNTHETIC AND NATURAL ESCHERICHIA COLI FREE LIPID A EXPRESS IDENTICAL ENDOTOXIC ACTIVITIES,” Eur. J. Biochem. 148:1-5). Being amphipathic and phosphorylated, lipid A and LPS are negatively charged phospholipids structurally resembling negatively charged glycero-phospholipids. In aqueous solutions, purified LPS forms stable high molecular weight homo-molecular aggregates. The basic structure of LPS (lipid A) suggests that LPS aggregates alone or as complexes with plasma proteins such as albumin or lipoproteins may be efficient ligands for SR-BI/CLA-1. Supportive evidence for this possibility comes from the observation that organs highly expressing CLA-1 are targeted by ¹²⁵I-LPS/HDL complexes, and the accumulation of LPS in the adrenal glands has been suggested to induce adrenal cortical insufficiency (Garcia, R. et al. (1990) “EFFECT OF ESCHERICHIA COLI ENDOTOXIN ON ASCORBIC ACID TRANSPORT IN ISOLATED ADRENOCORTICAL CELLS,” Proc. Soc. Exp. Biol. Med. 193:280-284; Garcia, R. et al. (11999) “CALCIUM AND REACTIVE OXYGEN SPECIES AS MESSENGERS IN ENDOTOXIN ACTION ON ADRENOCORTICAL CELLS,” Biochim Biophys Acta 1454: 1-10) or hemorrhage in septic patients (Mathison, J. C. et al. (1985) “UPTAKE AND SUBCELLULAR LOCALIZATION OF BACTERIAL LIPOPOLYSACCHARIDE IN THE ADRENAL GLAND,” Amer. J. Pathol. 120:79-86; Munford, R. S. et al. (1981) “SITES OF TISSUE BINDING AND UPTAKE IN VIVO OF BACTERIAL LIPOPOLYSACCHARIDE-HIGH DENSITY LIPOPROTEIN COMPLEXES: STUDIES IN THE RAT AND SQUIRREL MONKEY,” J. Clin. Invest. 68:1503-1513).

The present example clarifies the role of CLA-1 in LPS binding, uptake and intracellular transport when in lipoprotein-free form or in the association with HDL (purified HDLILPS complex). Lipoprotein-free LPS strongly competes with HDL and exchangeable lipid-poor HDL apolipoproteins for HDL-binding sites in RAW cells that highly express SR-BI/CLA-1 (Galanos, C. et al. (1985) “SYNTHETIC AND NATURAL ESCHERICHIA COLI FREE LIPID A EXPRESS IDENTICAL ENDOTOXIC ACTIVITIES,” Eur. J. Biochem. 148:1-5). Whereas LPS binding is measured on ice and in the absence of plasma factors facilitating LPS exchange, conditions highly unfavorable for HDL-LPS complex formation, the possibility of an effect of a small amount of HDL associated LPS on HDL-binding cannot be excluded. However, the competition of LPS against iodinated apolipoproteins, which has a much lower ability than HDL to interact with LPS, also supports the idea that HDL and LPS compete as independent ligands and share the same type of specific binding sites(s).

To evaluate human SR-BI/CLA-1 as a potential LPS-binding protein, a CLA-1 stably transfected HeLa cell line was created. Analyses of HDL-binding and selective cholesterol ester uptake were conducted to demonstrate functional CLA-1 activity in the cells. Both HDL-binding and cholesterol ester uptake were elevated by 10-fold in CLA-1 overexpressing HeLa cells when compared with a mock-transfected control. In addition, in an agreement with previous data on rodent SR-BI/CLA-1, its human orthologue, CLA-1, induced a 4-fold increase of the initial cholesterol efflux to HDL. CLA-1 overexpressing HeLa cells, demonstrated a 3-4 fold increase of the specific LPS binding with a Kd=16 μg/ml. It has been reported that LPS, an amphipathic molecule, exists in an aggregated form in aqueous buffers. Monomerization of LPS requires appropriate binding plasma proteins such as HDL or serum albumin (de Haas, C. J. et al. (2000) “ANALYSIS OF LIPOPOLYSACCHARIDE (LPS)-BINDING CHARACTERISTICS OF SERUM COMPONENTS USING GEL FILTRATION OF FITC-LABELED LPS,” J. Immunol. Methods 242:79-89) and a phospholipid transfer protein such as LBP. It has been reported that LPS association with lipoproteins at a low temperature or in the absence of serum factors such as LBP is inefficient (Yu, B. et al. (1997) “LIPOPOLYSACCHARIDE BINDING PROTEIN AND SOLUBLE CD14 CATALYZE EXCHANGE OF PHOSPHOLIPIDS,” J. Clin. Invest. 99:315-324). The present data demonstrates that the molecular mass of LPS when aggregated is approximately 1000 kDa as determined by fast protein liquid chromatography (FPLC) in the absence of BSA. Utilizing agarose gel electrophoresis it was surprisingly found that a 10-30-min incubation of Bodipy LPS or iodinated LPS in high BSA concentrations (1-20 mg/ml) monomerized LPS and resulted in forming BSA-LPS complexes even in the absence of serum phospholipid-transfer proteins. Assuming that LPS may form a monomolecular complex with BSA (Mw=75-80 kDa) or BSA dimers (Mw=150-160 kDa), the estimated Kd would be 1-2×10⁻⁷ M. This value is in the range of the Kd values previously determined for SR-BI/CLA-1 utilizing HDL or lipid poor exchangeable apolipoproteins (Xu, S. et al. (1997) “APOLIPOPROTEINS OF HDL CAN DIRECTLY MEDIATE BINDING TO THE SCAVENGER RECEPTOR SR-BI, AN HDL RECEPTOR THAT MEDIATES SELECTIVE LIPID UPTAKE,” J. Lipid Res. 38:1289-1298; Murao, K. et al. (1997) “CHARACTERIZATION OF CLA-1, A HUMAN HOMOLOGUE OF RODENT SCAVENGER RECEPTOR BI, AS A RECEPTOR FOR HIGH DENSITY LIPOPROTEIN AND APOPTOTIC THYMOCYTES,” J. Biol. Chem. 272:17551-17557). Because diverse LPS structures carry a highly conserved lipid A structure, the comparable potency among several forms of LPS, including DPLA, indicates that CLA-1 provides a binding site for the lipid A moiety of LPS rather than the highly variable polysaccharide O-antigen portion.

Earlier reports indicated that the expression of HDL-binding proteins, such as SR-BI/CLA-1 and ATP cassette transporters are under strict negative control by LPS-related activation of NF-kB in monocyte cell lines and the rodent liver (Buechler, C. et al. (1999) “LIPOPOLYSACCHARIDE INHIBITS THE EXPRESSION OF THE SCAVENGER RECEPTOR CLA-1 IN HUMAN MONOCYTES AND MACROPHAGES,” Biochem. Biophys. Res. Commun. 262:251-254; Khovidhunkit, W. et al. (2001) “REGULATION OF SCAVENGER RECEPTOR CLASS B TYPE I IN HAMSTER LIVER AND HEP3B CELLS BY ENDOTOXIN AND CYTOKINES,” J. Lipid Res. 42:1636-1644; Baranova, I. et al. (2002) “LIPOPOLYSACCHARIDE DOWN REGULATES BOTH SCAVENGER RECEPTOR B1 AND ATP BINDING CASSETTE TRANSPORTER A1 IN RAW CELLS,” Infect. Immun. 70:2995-3003). Because the decreased endogenous level of CLA-1 or ABCA1 expression might be a confounding factor for the differences in LPS uptake in HeLa cells, the levels of CLA-1, IL-6, IL-8 and IL-1 in HeLa cells after LPS-stimulation are measured. In agreement with previous reports on HeLa cell's unresponsiveness to LPS (Thieblemont, N. et al. (1999) “TRANSPORT OF BACTERIAL LIPOPOLYSACCHARIDE TO THE GOLGI APPARATUS,” J. Exp. Med. 190:523-534), the results indicate that there is no LPS effect when using a wide range of concentrations upon both IL-6 and IL-8 secretion as well as CLA-1 expression by ELISA and Western blotting, respectively.

It has been reported that CD14 associated LPS after initially binding in caveolae is transported to the Golgi complex (Khovidhunkit, W. et al. (2001) “REGULATION OF SCAVENGER RECEPTOR CLASS B TYPE I IN HAMSTER LIVER AND HEP3B CELLS BY ENDOTOXIN AND CYTOKINES,” J. Lipid Res. 42:1636-1644). It is significant that SR-BI/CLA-1 is predominantly associated with caveolae (Babitt, J. et al. (1997) “MURINE SR-BI, A HIGH DENSITY LIPOPROTEIN RECEPTOR THAT MEDIATES SELECTIVE LIPID UPTAKE, IS N-GLYCOSYLATED AND FATTY ACYLATED AND COLOCALIZES WITH PLASMA MEMBRANE CAVEOLAE,” J. Biol. Chem. 272:13242-13249), which have been demonstrated as the initial loci for membrane transfer of HDL cholesteryl esters (Graf, G. A. et al. (1999) “THE CLASS B, TYPE I SCAVENGER RECEPTOR PROMOTES THE SELECTIVE UPTAKE OF HIGH DENSITY LIPOPROTEIN CHOLESTEROL ETHERS INTO CAVEOLAE,” J. Biol. Chem. 274:12043-12048), sphingomyelin (SM), phosphatidylcholine, and phosphatidyl-ethanolamine (Urban, S. et al. (2000) “SCAVENGER RECEPTOR BI TRANSFERS MAJOR LIPOPROTEIN-ASSOCIATED PHOSPHOLIPIDS INTO THE CELLS,” J. Biol. Chem. 275:33409-33415). It has been also been reported that SR-BI/CLA-1 is directly involved with caveolae-like microvilliar channel formation in a heterologous SR-BI/CLA-1 expressing cell system (Reaven, E. et al. (2001) “EXPRESSION OF SCAVENGER RECEPTOR CLASS B TYPE 1 (SR-BI) PROMOTES MICROVILLAR CHANNEL FORMATION AND SELECTIVE CHOLESTERYL ESTER TRANSPORT IN A HETEROLOGOUS RECONSTITUTED SYSTEM,” Proc. Natl. Acad. Sci. U.S.A 98:1613-1618).

In an agreement with the data on specific ¹²⁵I-LPS binding (FIGS. 9A, 9B, and 9C), cellular ¹²⁵I-LPS-uptake followed by LPS internalization is dramatically increased in CLA-1 overexpressing HeLa cells when evaluated at 37° C. In contrast to mock-transfected HeLa cells, where the amount of internalized ligand decreased significantly by the third hour of incubation, cell associated ¹²⁵I-LPS remained steady after plateauing at 60-min. It is observed that CLA-1 overexpression in HeLa cells (LPS-non responsive cells) did not affect NF-KB responsive genes as estimated by RT-PCR and ELISA detection of IL-6, IL-8 and TNF-α in culture media. However, CLA-1 can also regulate caveolae structure as well as surface clustering of LPS/membrane CD14 and TLR4, which can affect LPS signaling.

Previous studies suggested that CLA-1 might be involved with LPS efflux, the process of dissociation of LPS from the cell surface to HDL particles (Kitchens, R. L. et al. (1999) “PLASMA LIPOPROTEINS PROMOTE THE RELEASE OF BACTERIAL LIPOPOLYSACCHARIDE FROM THE MONOCYTE CELL SURFACE,” J. Biol. Chem. 274:34116-34122). This process is similar to passive cholesterol efflux, which is increased in mouse SR-BI/CLA-1 overexpressing cells, and which might be patho-physiologically significant during endotoxemia (Kitchens, R. L. et al. (1999) “PLASMA LIPOPROTEINS PROMOTE THE RELEASE OF BACTERIAL LIPOPOLYSACCHARIDE FROM THE MONOCYTE CELL SURFACE,” J. Biol. Chem. 274:34116-34122). However, LPS-release from the cell surface could also be the result of direct competition between HDL and LPS for LPS/HDL-binding sites. It is observed that apoA-I, a CLA-1 ligand; at 100× excess, eliminated surface bound Bodipy-LPS in CLA-1 overexpressing cells. Moreover, when added at the same concentration, Alexa-apoA-1 and Bodipy-LPS co-localized on the cell surface, suggesting a common binding site on the plasma membrane. Both ligands were predominantly transported into a perinuclear compartment, which was determined to be the Golgi network by a co-localization with ceramide-BSA complex. The finding of surface bound LPS being intracellularly transported to the Golgi complex is consistent with previous reports that LPS is rapidly transported to a perinuclear compartment, which has been identified as the Golgi complex (Thieblemont, N. et al. (1999) “TRANSPORT OF BACTERIAL LIPOPOLYSACCHARIDE TO THE GOLGI APPARATUS,” J. Exp. Med. 190:523-534). Regarding the novel observation of the rapid endocytosis and intracellular transport of lipid poor apoA-1, no reference for this observation has been found in the literature. Importantly a human monocyte cell line, THP-1 that expresses a high level of SR-BI/CLA-1, also demonstrated a rapid Alexa-568 apoA-1 endocytosis. This indicates that apoA-1 perinuclear transportation is not an artifact of high CLA-1 expression or the result of the use of a particular cell model. It is therefore concluded that CLA-1 related apoA-1 binding and endocytosis can be one of the mechanisms involved with the accelerated apoA-1 clearance reported for Tangier disease (Schaefer, E. J. et al. (1981) “METABOLISM OF HIGH DENSITY LIPOPROTEIN SUBFRACTIONS AND CONSTITUENTS IN TANGIER DISEASE FOLLOWING THE INFUSION OF HIGH DENSITY LIPOPROTEINS,” J. Lipid Res. 22:217-228). The significant intracellular co-localization of LPS and apoA-1 also suggests the same intracellular transporting mechanism(s) for both ligands. These observations also suggest that the effects of HDL and apoA-I infusion during endotoxemia, which have been shown to have a beneficial effect (Levine, D. M. et al. (1993) “IN VIVO PROTECTION AGAINST ENDOTOXIN BY PLASMA HIGH DENSITY LIPOPROTEIN,” Proc. Natl. Acad. Sci. U.S.A 90:12040-12044), could partially result from the direct competition between SR-BI ligands and LPS cellular binding sites. If there is binding competition, other CLA-1 related synthetic agonists; such as the double a-helical amphipathic peptides resembling the exchangeable apolipoproteins AI and A-II could be evaluated as potential treatments of septic shock. Supporting this possibility is an earlier study which demonstrated that phospholipid vesicles reconstituted with a 18A single ahelical amphipathic peptide as well as structurally related a-helical cationic antimicrobial peptides prevented LPS-related mortality in a murine model (Levine, D. M. et al. (1993) “IN VIVO PROTECTION AGAINST ENDOTOXIN BY PLASMA HIGH DENSITY LIPOPROTEIN,” Proc. Natl. Acad. Sci. U.S.A 90:12040-12044).

It appears that lipid transport and LPS neutralization utilize similar mechanisms. Recently it has been shown that LBP together with cholesterol ester transfer protein (CETP) and phospholipid transfer protein (PL TP), the major proteins involved with HDL remodeling, belong to the same family of lipid transport proteins (Hailman, E. et al. (1996) “NEUTRALIZATION AND TRANSFER OF LIPOPOLYSACCHARIDE BY PHOSPHOLIPID TRANSFER PROTEIN,” J. Biol. Chem. 271:12172-12178). A number of observations demonstrate that LPS association with HDL is an important step in LPS neutralization and clearance. Generally, LPS clearance from plasma is enhanced when LPS is associated with lipoproteins and results in increased biliary excretion (Read, T. E. et al. (1993) “THE PROTECTIVE EFFECT OF SERUM LIPOPROTEINS AGAINST BACTERIAL LIPOPOLYSACCHARIDE,” Eur. Heart J. 14 Suppl K, 125-129).

The present data demonstrates that the LPS uptake from an HDL/LPS complex is significantly increased in CLA-1 overexpressing HeLa cells when compared with mock transfected HeLa cells. Similarly to selective CE and lipid uptake (Thuahnai, S. T. et al. (2001) “SCAVENGER RECEPTOR CLASS B, TYPE I-MEDIATED UPTAKE OF VARIOUS LIPIDS INTO CELLS. INFLUENCE OF THE NATURE OF THE DONOR PARTICLE INTERACTION WITH THE RECEPTOR,” J. Biol. Chem. 276:43801-43808, Urban, S. et al. (2000) “SCAVENGER RECEPTOR BI TRANSFERS MAJOR LIPOPROTEIN-ASSOCIATED PHOSPHOLIPIDS INTO THE CELLS,” J. Biol. Chem. 275:33409-33415), radiolabeled LPS are taken up at a higher rate than radiolabeled HDL apolipoproteins, indicating that selective LPS uptake takes place. Secreted HDL apolipoproteins are partially degraded in Cla-1 overexpressing cells, indicating that some portion of HDL may be delivered to lysozymes. The ratio of degraded versus resecreted HDL apolipoprotein is observed to be increased when the uptake of HDL.LPS complex was examined (FIGS. 11C and 11D). It is possible that such an increase in HDL uptake and degradation may be one of the factors causing HDL levels to decrease during septic conditions. By utilizing confocal microscopy, CLA-1 overexpressing HeLa cells rapidly bind and internalize Bodipy-LPS/Alexa488-protein labeled HDL as a holoparticle into a peri-nuclear compartment. Upon longer incubations, LPS and apolipoproteins were sorted to distinct intracellular compartments that are clearly visible on confocal images by the segregation of green (HDL) and red (LPS) signals. SR-BI/CLA-1 has been thought to mediate a selective uptake of high-density lipoprotein (HDL) cholesteryl ester without the uptake and degradation of the particle. In contrast to its accepted role as a surface functioning, non-endocytic receptor, rodent SR-BI/CLA-1 has been recently shown to mediate HDL particle uptake, endocytosis and lipid sorting in both transfected Chinese hamster ovary cells and hepatocytes (Silver, D. L. et al. (2001) “HIGH DENSITY LIPOPROTEIN (HDL) PARTICLE UPTAKE MEDIATED BY SCAVENGER RECEPTOR CLASS B TYPE 1 RESULTS IN SELECTIVE SORTING OF HDL CHOLESTEROL FROM PROTEIN AND POLARIZED CHOLESTEROL SECRETION,” J. Biol. Chem. 276:25287-25293). Internalized HDL particles enter the endocytic recycling compartment paralleling the movement of rodent SR-BI/CLA-1 (Silver, D. L. et al. (2001) “HIGH DENSITY LIPOPROTEIN (HDL) PARTICLE UPTAKE MEDIATED BY SCAVENGER RECEPTOR CLASS B TYPE 1 RESULTS IN SELECTIVE SORTING OF HDL CHOLESTEROL FROM PROTEIN AND POLARIZED CHOLESTEROL SECRETION,” J. Biol. Chem. 276:25287-25293). In agreement with these data, Cla-1 overexpressing HeLa cells rapidly internalize both Alexa 568 apolipoprotein-labeled HDL and HDL-bound Bodipy-LPS. Lipoprotein bound label is transported to the endocytic recycling compartment as determined by colocalization experiments utilizing Bodipy-transferrin. In contrast to BSA-monomerized LPS, HDL-bound Bodipy-LPS does not significantly co-localize with the Bodipy-ceramide BSA complex. This indicates that an association of LPS with HDL results in re-compartmentalization of LPS taken up by the Cla-1 receptor from the Golgi complex to the endocytic recycling compartment.

In summary, the data demonstrate that Cla-1, a known HDL receptor involved with the trafficking of lipids and lipid-like molecules, is a potent LPS-binding protein, which mediates LPS binding and endocytosis. Lipoprotein-free LPS serves as an independent ligand like other SR-BI/CLA-1 ligands including HDL, apoA-I, and apoA-II. Cla-1 expression dramatically increases the uptake, internalization, and intracellular accumulation of LPS associated with HDL in a process closely resembling HDL cholesterol ester uptake and intracellular sorting. These data strongly indicate that Cla-1 is an important mechanism of liver LPS uptake and bile secretion. Following up these findings leads to multiple achievements, including the role of Cla-1 in LPS-induced cortical insufficiency and direct toxicity in adrenal glands, LPS-mediated signaling and LPS clearance by the liver. Knowledge regarding the functional relationship between of Cla-1 and LPS permits the development of new treatments for sepsis and septic shock.

Thus, the present Example demonstrates that scavenger receptor, class B, type I (SR-BI/CLA-1) mediates selective uptake of high-density lipoprotein (HDL) cholesteryl ester. In transfected cells, SR-BI/CLA-1 recognizes multiple ligands including HDL, low-density lipoprotein (LDL), exchangeable apolipoproteins and protein-free lipid vesicles containing negatively charged phospholipids. Lipopolysaccharides (LPS) are highly glycosylated anionic phospholipids that are implicated in the pathogenesis of, and contribute to, septic shock (Vishnayakova, T. et al. (2003) “BINDING AND INTERNALIZATION OF LIPOPOLYSACCHARIDE BY CLA-1, A HUMAN ORTHOLOGUE OF RODENT SCAVENGER RECEPTOR BI,” J. Biol. Chem. 25:22771-22780 (2003), herein incorporated by reference in its entirety). Despite the existence of significant structural similarities between anionic phospholipids and LPS, the role of SR-BI/CLA-1 in LPS-uptake has not been reported. The above-described report demonstrates for the first time that CLA-1, the human SR-BI/CLA-1 orthologue, functions as a lipopolysaccharide (LPS)-binding protein and endocytic receptor mediating the binding and internalization of lipoprotein-free, bovine serum albumin (BSA) monomerized LPS. LPS strongly competes with HDL, lipid-free apoA-1 and apoA-Ii for HDL-binding to the mouse RAW 264.7 monocyte cell line. Stably transfected HeLa cells expressing CLA-1 bind LPS with a Kd of about 16 μg/ml and have a 4-fold increase in binding capacity. Glycosylated LPS (0111:B4), S. Minnesota 595 Re LPS, as well as lipid A, all compete for ¹²⁵I-HDL-binding to CLA-1 overexpressing human HeLa cells. In addition to increased binding, there is a 3-4-fold increase in LPS uptake in CLA-1 overexpressing cells compared to control HeLa cells. Bodipy-labeled LPS uptake is found to accumulate initially in the plasma membrane and subsequently in a perinuclear region identified predominantly as the Golgi complex. Bodipy-LPS and Alexa-apoA-I, an SR-BI/CLA-1 ligand, have staining which co-localized on the cell surface and intracellularly indicating similar transport mechanisms for both ligands. When associated with HDL, LPS uptake was increased in CLA-1 overexpressing HeLa cells by 5-10 fold indicating a selective LPS uptake and the intracellular transport in CLA-1 overexpressing HeLa cells when compared with mock-transfected HeLa cells. Upon interacting with CLA-1 overexpressing HeLa cells, the complex (Bodipy-LPS/Alexa 488 apolipoprotein labeled HDL) binds and is internalized as a holoparticle. Intracellularly, LPS and apolipoproteins are sorted to different intracellular compartments. With LPS associated HDL, intracellular LPS colocolized predominantly with transferring indicating delivery to an endocytic recycling compartment. The data reveal a close similarity between CLA-1 mediated selective LPS uptake and the selective cholesterol sorting by rodent SR-BI/CLA-1. CLA-1 was found to bind as well as internalize BSA-monomerized and HDL-associated LPS. These data indicate that CLA-1 plays an important role in septic shock by affecting LPS cellular uptake and clearance.

In sum, scavenger receptor, class B, type I (SR-BI/CLA-1), is an HDL-receptor, which mediates the selective uptake of high-density lipoprotein (HDL) cholesteryl ester without the uptake and degradation of the particle. SR-BI/CLA-1 ligand's recognizing motif includes the class A amphipathic-helix of exchangeable apolipoproteins and anionic gycerophospholipids. Lipopolysaccharides (LPS) are highly glycosylated anionic disaccharide based phospholipids that are implicated in the pathogenesis of septic shock. Despite the existence of strong structural similarities between anionic phospholipids and LPS, the role of SR-BI/CLA-1 in LPS-uptake is unknown. The present invention demonstrates for the first time that CLA-1, human SR-BI/CLA-1, functions as a lipopolysaccharide (LPS)-binding protein and mediates binding and internalization of LPS. LPS strongly competes with HDL, lipid-free apoA-I and apoA-II for HDL-binding sites in the mouse RAW 264.7 monocyte cell line. Stably transfected Hela cells overexpressing CLA-1 bind LPS with a Kd of about 16 μg/ml and an over 4-fold increased capacity. Glycosylated LPS (0111:B4), S. Minnesota 595 Re LPS, as well as lipid A, all competed for 125I-HDL-binding to CLA-1 overexpressing human HeLa cells. In addition to increased binding, there was a 3-4-fold increase in LPS uptake in CLA-1 overexpressing cells compared to control Hela cells. Bodipy-labeled LPS uptake was found to accumulate in the plasma membrane and in the peri-nuclear region of CLA-1 expressing Hela cells. Both Bodipy-LPS and Alexa 568-HDL as well as Bodipy-LPS and anti-Cla-1 staining were co-localized intracellularly and on the cell surface. The Bodipy-LPS/Cla-1 cross-linking product had molecular weight of 90 kDa and was co-precipitated with an anti Cla-1 antibody. In summary, Cla-1 functions as LPS-receptor mediating both binding and internalization of LPS. The observation of LPS and Cla-1 co-localization as well as a 90 kDa cross-linking product suggest that Cla-1 may play an important role in septic shock by affecting LPS clearance.

### Example 3

### Synthetic Amphipathic α-Helical Peptides Mimic of Exchangeable Apolipoproteins Block LPS uptake and LPS-Induced Proinflammatory Cytokine Response by THP-1 Monocyte Cells

Lipopolysaccharides (LPS) are proinflammatory bacterial cell wall components implicated in the pathogenesis of gram-negative sepsis and septic shock. The Examples provided above demonstrate that human scavenger receptor class B type I (CLA-1) mediates LPS-binding and internalization in overexpressing HeLa cells. Since the major recognition motif in SR-BI/CLA-1 ligands is an amphipathic α helix, the purpose of this study was to analyze the effects of synthetic peptides, which mimic anti-atherogenic exchangeable apolipoproteins, on LPS-uptake and LPS-stimulated cytokine production in HeLa and THP-1 cells, respectively. The L-37PA peptide which contains two class A amphipathic a-helices linked by proline efficiently competed against iodinated LPS in both mock transfected and CLA-1 overexpressing HeLa cells. A 100-fold excess of L37PA diminished Bodipy LPS uptake in the cells, blocking both LPS-binding to the plasma membrane and LPS internalization. The L-37PA as well as D-37PA peptide, synthesized with D-amino acids, was similarly effective in a blocking LPS-stimulated IL-B, IL˜8 and TNFα gene expression and the cytokine secretion in THP-1 cells and human fibroblasts. In contrast, neither peptides was effective in blocking of TNFα-induced IL-B and IL-8 production in THP-1 cells. Peptides containing only a single helix (18A) and a peptide that contains a mixture of Land D amino acids (L2D-37PA) do not form helices, did not affect LPS-uptake and LPS-stimulated cytokine production in both cells. L37P A, 18A and L2D-37P A similarly neutralized LPS activity in the Limulus amebocyte lysate (LAL) test in the absence of divalent cations. However they had no effect on the LAL activity of LPS in culture media used for cytokine stimulation study, indicating that the blocking effects of L-37PA are not related to LPS-neutralization activity. In summary, synthetic amphipathic helical peptides (L37PA and D-37PA) that bind CLA-1 block LPS uptake and LPS-induced proinflammatory response.

### Example 4

### Targeting of Scavenger Receptor Class B Type I by Synthetic Amphipathic α-Helical Containing Peptides Blocks LPS Uptake and LPS-Induced Proinflammatory Cytokine Responses in THP-1 Monocyte Cells

As indicated above, LPS-binding to cell receptor(s) causes a proinflammatory cellular response as well as mediates degradation and clearance of endotoxins. Recently, it has been demonstrated that LPS-induced cytokine response primarily involves Toll Like Receptor 4 (TLR4) and plasma membrane CD14, which initialize down stream signaling to NF-kB followed by activation of LPS-responsive genes including proinflammatory cytokines (Lien, E. et al. (2000) “TOLL-LIKE RECEPTOR 4 IMPARTS LIGAND-SPECIFIC RECOGNITION OF BACTERIAL LIPOPOLYSACCHARIDE,” J. Clin. Invest 105:497-504, Chow, J. C. et al. (1999) “TOLL-LIKE RECEPTOR-4 MEDIATES LIPOPOLYSACCHARIDE-INDUCED SIGNAL TRANSDUCTION,” J. Biol. Chem. 274:10689-10692). LPS-uptake, clearance and catabolism is mediated by a family of scavenger receptors (van Oosten, M. et al. (2001) “SCAVENGER RECEPTOR-LIKE RECEPTORS FOR THE BINDING OF LIPOPOLYSACCHARIDE AND LIPOTEICHOIC ACID TO LIVER ENDOTHELIAL AND KUPFFER CELLS,” J. Endotoxin Res. 7:381-384; Shnyra, A. et al. (1995) “SCAVENGER RECEPTOR PATHWAY FOR LIPOPOLYSACCHARIDE BINDING To KUPFFER AND ENDOTHELIAL LIVER CELLS IN VITRO,” Infect. Immun. 63:865-873; Seternes, T. et al. (2001) “SCAVENGER-RECEPTOR-MEDIATED ENDOCYTOSIS OF LIPOPOLYSACCHARIDE IN ATLANTIC COD (GADUS MORHUA L.),” J. Exp. Biol. 204:4055-4064; Hampton, R Y. et al. (1991) “RECOGNITION AND PLASMA CLEARANCE OF ENDOTOXIN BY SCAVENGER RECEPTORS,” Nature 352:342-344). LPS-binding to class A scavenger receptors, which is associated with classical clathrindependent internalization and delivery to lysosomal compartments, has been demonstrated however, to only have a minor role in LPS clearance in vivo (van Oosten, M. et al. (2001) “SCAVENGER RECEPTOR-LIKE RECEPTORS FOR THE BINDING OF LIPOPOLYSACCHARIDE AND LIPOTEICHOIC ACID TO LIVER ENDOTHELIAL AND KUPFFER CELLS,” J. Endotoxin Res. 7:381-384).

In vitro cellular LPS-uptake is mediated by several unrelated mechanisms including formation of clathrin-coated vesicles (Kang, Y. H. et al. (1990) “ULTRASTRUCTURAL AND IMMUNOCYTOCHEMICAL STUDY OF THE UPTAKE AND DISTRIBUTION OF BACTERIAL LIPOPOLYSACCHARIDE IN HUMAN MONOCYTES,” J. Leukoc. Biol. 48:316-332); macropinocytosis (Poussin, C. et al. (1998) “CD14-DEPENDENT ENDOTOXIN INTERNALIZATION VIA A MACROPINOCYTIC PATHWAY,” J. Biol. Chem. 273:20285-20291); and via uncoated plasma membrane invaginations involving caveolae and micropinocytosis (Poussin, C. et al. (1998) “CD14-DEPENDENT ENDOTOXIN INTERNALIZATION VIA A MACROPINOCYTIC PATHWAY,” J. Biol. Chem. 273:20285-20291, Kriegsmann, J. et al. (1993) “ENDOCYTOSIS OF LIPOPOLYSACCHARIDE IN MOUSE MACROPHAGES,” Cell Mol. Biol. 39:791-800). The aggregation state of LPS determines the mechanism of LPS-uptake. Aggregated LPS has been demonstrated to enter cells through clathrin-coated pits. Monomerized LPS is internalized by a mechanism involving uncoated plasma membrane invaginations and subsequently transported to the Golgi complex (Thieblemont, N. et al. (1999) “TRANSPORT OF BACTERIAL LIPOPOLYSACCHARIDE TO THE GOLGI APPARATUS,” J. Exp. Med. 190:523-534).

The role of rafts and the Golgi complex in LPS-induced signaling is intensively being investigated since the major LPS-signaling receptor, TLR4, resides within the Golgi network and plasma membrane rafts (Latz, E. et al. (2002) “LIPOPOLYSACCHARIDE RAPIDLY TRAFFICS TO AND FROM THE GOLGI APPARATUS WITH THE TOLL-LIKE RECEPTOR 4-MD-2-CD14 COMPLEX IN A PROCESS THAT IS DISTINCT FROM THE INITIATION OF SIGNAL TRANSDUCTION,” J. Biol. Chem. 277:47834-47843, Hornef, M. W. et al. (2002) “TOLL-LIKE RECEPTOR 4 RESIDES IN THE GOLGI APPARATUS AND COLOCALIZES WITH INTERNALIZED LIPOPOLYSACCHARIDE IN INTESTINAL EPITHELIAL CELLS,” J. Exp. Med. 195:559-570). Interestingly, in the intestinal epithelial cell TLR4 does not appear on the plasma membrane, but rather is exclusively found in the Golgi complex. Furthermore in these cells, LPS induced signaling requires LPS-internalization (Latz, E. et al. (2002) “LIPOPOLYSACCHARIDE RAPIDLY TRAFFICS TO AND FROM THE GOLGI APPARATUS WITH THE TOLL-LIKE RECEPTOR 4-MD-2-CD14 COMPLEX IN A PROCESS THAT IS DISTINCT FROM THE INITIATION OF SIGNAL TRANSDUCTION,” J. Biol. Chem. 277:47834-47843, Hornef, M. W. et al. (2002) “TOLL-LIKE RECEPTOR 4 RESIDES IN THE GOLGI APPARATUS AND COLOCALIZES WITH INTERNALIZED LIPOPOLYSACCHARIDE IN INTESTINAL EPITHELIAL CELLS,” J. Exp. Med. 195:559-570). Intestinal epithelial cells, hepatocytes as well as antigen presenting dendritic cells highly express class B scavenger receptors, such as CLA-1, which have recently been shown to bind, internalize and transport LPS to the Golgi (Vishnyakova, T. G. et al. (2003) “BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780).

Human scavenger receptor, class B, Type I (SR-BI/CLA-1) and its human orthologue CD36 and LIMPII analog-1 (CLA-1), are plasma membrane proteins, which function as HDL-receptors (Babitt, J. et al. (1997) “MURINE SR-BI, A HIGH DENSITY LIPOPROTEIN RECEPTOR THAT MEDIATES SELECTIVE LIPID UPTAKE, IS N-GLYCOSYLATED AND FATTY ACYLATED AND COLOCALIZES WITH PLASMA MEMBRANE CAVEOLAE,” J. Biol. Chem. 272:13242-13249). SR-BI/CLA-1 colocalizes with caveolin, and found in the triton X-100 insoluble membrane fraction indicating its involvement with raft and caveolae raft formation (Reaven, E. et al. (2001) “EXPRESSION OF SCAVENGER RECEPTOR CLASS B TYPE 1 (SR-BI) PROMOTES MICROVILLAR CHANNEL FORMATION AND SELECTIVE CHOLESTERYL ESTER TRANSPORT IN A HETEROLOGOUS RECONSTITUTED SYSTEM,” Proc. Natl. Acad. Sci. U.S.A 98:1613-1618). In transfected mammalian cells SR-BI/CLA-1 induces a marked increase in HDL-binding, HDL cholesteryl ester uptake and the translocation of the major structural component of the plasma membrane caveolae, caveolin-1, to the cell surface (Matveev, S. et al. (2001) “CAVEOLIN-1 NEGATIVELY REGULATES SR-BI MEDIATED SELECTIVE UPTAKE OF HIGH-DENSITY LIPOPROTEIN-DERIVED CHOLESTERYL ESTER,” Eur. J Biochem. 268:5609-5616). Plasma membrane caveolae (rafts) have been recently demonstrated as the initial loci for the membrane transfer of HDL lipids (Graf, G. A. et al. (1999) “THE CLASS B, TYPE I SCAVENGER RECEPTOR PROMOTES THE SELECTIVE UPTAKE OF HIGH DENSITY LIPOPROTEIN CHOLESTEROL ETHERS INTO CAVEOLAE,” J. Biol. Chem. 274:12043-12048) as well as for cell signaling (Latz, E. et al. (2002) “LIPOPOLYSACCHARIDE RAPIDLY TRAFFICS TO AND FROM THE GOLGI APPARATUS WITH THE TOLL-LIKE RECEPTOR 4-MD-2-CD14 COMPLEX IN A PROCESS THAT IS DISTINCT FROM THE INITIATION OF SIGNAL TRANSDUCTION,” J. Biol. Chem. 277:47834-47843).

Negatively charged phospholipids and anionic class A amphipathic α-helixes of exchangeable apolipoproteins serve as two primary recognition motifs upon HDL interaction with SR-BI/CLA-1 (CLA-1) (Schulthess, G. et al. (2000) “INTESTINAL STEROL ABSORPTION MEDIATED BY SCAVENGER RECEPTORS IS COMPETITIVELY INHIBITED BY AMPHIPATHIC PEPTIDES AND PROTEINS,” Biochemistry 39:12623-12631, Williams, D. L. et al. (2000) “BINDING AND CROSS-LINKING STUDIES SHOW THAT SCAVENGER RECEPTOR BI INTERACTS WITH MULTIPLE SITES IN APOLIPOPROTEIN A-I AND IDENTIFY THE CLASS A AMPHIPATHIC ALPHA-HELIX As A RECOGNITION MOTIF,” J. Biol. Chem 275:18897-18904). It has been demonstrated that short-length synthetic amphipathic helical peptides which resemble class A amphipathic α-helixes of exchangeable apolipoproteins as well as LPS, which is an anionic glucosamine-based phospholipid, bind to CLA-1 with high affinity (Vishnyakova, T. G. et al. (2003) “BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780).

Since CLA-1 associates with rafts and transports LPS to the Golgi, the two sites of TLR localization, it was hypothesized that targeting SR-BI/CLA-1 with synthetic amphipathic helical peptides might affect LPS-induced cytokine expression by competing for the SR-BI/CLA-1. In this study we investigated the effect of L-37PA and D-37PA, which are class A helical peptides, on LPS binding, internalization and LPS-induced cytokine production in HeLa cells and human monocyte THP-1 cells. The present Example explores this hypothesis.

### Materials and Methods

Lipopolysacharides. Escherichia coli 0111:B4, Salmonella minnesota Re 595, LTA and Gro-EL were purchased from Sigma. Rabbit anti-SR-BI/CLA-1 antibody crossreacting with the human orthologue, CLA-1 was from Novus Biological. All fluorescent probes and labels were from Molecular Probes.

Synthesis of Amphipathic Helical Peptides. The peptides are synthesized by a solid-phase procedure as described by Merrifield, R B. (1969) (“SOLID-PHASE PEPTIDE SYNTHESIS,” Adv. Enzymol. Relat Areas Mol. Biol. 32:221-296) and Fairwell, T. et al. (1987) (“HUMAN PLASMA APOLIPOPROTEIN C-II: TOTAL SOLID-PHASE SYNTHESIS AND CHEMICAL AND BIOLOGICAL CHARACTERIZATION,” Proc. Natl. Acad. Sci. U.S. A 84:4796-4800). The sequences of peptides used in the study are as indicated in Table 1.

TABLE 1

Peptide Sequences
(with D-Amino Acid residues underlined)

| | | | |
|---|---|---|---|
| Peptide | SEQ ID | | |
| # | NO: | Peptide | Sequence |

| | | | |
|---|---|---|---|
| 1 | 9 | 18A | DWLKAFYDKVAEKLKEAF |
| 2 | 10 | L-37PA | DWLKAFYDKVAEKLKEAF-P- |
| | | | DWLKAFYDKVAEKLKEAF |
| 3 | 11 | D-37PA | DWLKAFYDKVAEKLKEAF-P- |
| | | | DWLKAFYDKVAEKLKEAF |
| 4 | 12 | L1D-37PA | DWLKAFYDKVAEKLKEAF-P- |
| | | | DWLKAFYDKVAEKLKEAF |
| 5 | 13 | L2D-37PA | DWLKAFYDKVAEKLKEAF-P- |
| | | | DWLKAFYDKVAEKLKEAF |
| 6 | 14 | L3D-37PA | DWLKAFYDKVAEKLKEAF-P- |
| | | | DWLKAFYDKVAEKLKEAF |

THP-1 and CLA-1 overexpressing HeLa cells. Human monocyte-macrophages, THP-1 (ATCC [American Type Culture Collection] TIB 71), are grown in 48-well plates in RPMI 1640 medium supplemented with 10% fetal calf serum (FCS), penicillin (100 U/ml), and streptomycin (100 μg/ml). All experiments involving LPS-induced interleukin production utilize the same media except that 1% FCS was used. Hela cells were cultured as previously reported (Vishnyakova, T. G. et al. (2003) “BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780).

HDL, Apolipoprotein Isolation. Human apolipoprotein E free HDL2+3 and apolipoproteins are isolated from the plasma of healthy donors as reported by Vishnyakova, T. G. et al. (2003) (“BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780).

¹²⁵I-LPS-Binding Assay. The LPS from E. coli 0111:B4 (Sigma) is iodinated as reported by Ulevitch, R J. (1978) (“THE PREPARATION AND CHARACTERIZATION OF A RADIOIODINATED BACTERIAL LIPOPOLYSACCHARIDE,” Immunochemistry 15:157-164). HeLa cells grown until 70% confluence in DMEM with 10% FCS are washed with PBS and cultured for 24 hr in serum-free DMEM. After chilling on ice, cells are incubated in the presence of 1 μg/ml ¹²⁵I-LPS and increasing concentrations of cold ligands (LPS, L-37PA and D-37PA) for 1 h. After washing with ice-cold PBS, the cells are hydrolyzed in 1 N NaOH. Radioactivity is counted in an LKB Wallac Ultragamma counter.

Preparation of BODIPY-LPS and Alexa-568 HDL, Lipid-free ApoA-I, L-37PA, L2D-37PA and 18A. HDL, apoA-I, L-37PA, L2D-37PA and 18A are conjugated with Alexa-568, SE (Molecular Probes, protein labeling kit) following the kit instructions. The Alexa ligands are analyzed by 10-20% Tricine-SDS peptide gel electrophoresis. Gels are scanned using Variable Mode Imager, Typhoon 9200, Molecular Dynamics. Alexa-labeled preparations of HDL, apolipoproteins and the peptides are found in appropriate positions with molecular masses of 28, 5 and 2.5 kDa for apoA-I, L-37PA and 18PA, respectively. S. minnesota Re-LPS was labeled using the BODIPY*FL, SE labeling kit from Molecular Probes, Inc. (Eugene, Oreg.) following the manufacturer's suggested procedure and modifications reported by Levels, J. H. et al. (2001) (“DISTRIBUTION AND KINETICS OF LIPOPROTEIN-BOUND ENDOTOXIN,” Infect. Immun. 69, 2821-2828).

Limulus Amebocyte Lysate (LAL) Assay for LPS. The LAL activity of LPS incubated with various peptides is quantitatively determined by a chromogenic Limulus amebocyte lysate test (Kinetic-QCL, BioWhittaker, Walkersville, Md.). The assay is carried out as recommended by the manufacturer and had an analytical sensitivity of 0.005 EU/ml (˜0.5 μg highly purified LPS/ml).

Uptake of BODIPY-LPS and Alexa 568 HDL, L-37PA/LPS and ApoA-I/LPS Co-localization Experiments. HeLa cells cultured on glass micro-slides are incubated with 5 μg/ml of Alexa568 HDL, 1-0.5 μg/ml of Alexa 568 apoA-I, 1-0.5 μg/ml of Alexa 568 L-37PA or 0.5 μg/ml Bodipy-LPS for 1-2 h in a CO2 incubator in DMEM containing 1 mg/ml BSA. The effect of L-37PA on LPS uptake is studied by incubating HeLa cells with 0.5 μg/ml Bodipy-LPS in the presence of 100 μg/ml of L-37PA for 1-2 h in a CO₂ incubator. For co-localization, Bodipy-LPS and Alexa 568-HDL or Bodipy-LPS and Alexa 568-L-37PA were used at the same concentrations of 0.5 μg/ml. Fluorescence was viewed with a Zeiss 510 laser scanning confocal microscope, using a krypton-argon-Omnichrome laser with excitation wavelengths of 488 and 568 nm for Bodipy-LPS and Alexa-568 labels, respectively.

Sites of LPS, L-37PA, ApoA-I Transport in CLA-1 Overexpressing HeLa Cells. For studying the sites of LPS, L-37PA and apoA-I delivery, cells are incubated with 1 μg/ml Bodipy-LPS, 1 μg/ml Alexa 568 L-37PA or 5 μg/ml Alexa 568-apoA-I at 37° C. for 2 h, then washed and chased at 37° C. for 30 minutes in the presence of Bodipy-transferrin or Bodipy-ceramide BSA complex.

Assays for cytokines and lactate dehydrogenase (LD). Interleukins 6 and 8 (IL-6 and IL-8) and tumor-necrosis factor-α (TNF-α) are measured in culture supernatants of THP-1 cells using commercial ELISA kits (Biosource International, USA). LD activity was measured in the supernatants by a Hitachi 917 automated chemistry analyzer (Roche).

Detection of mRNA by RT-PCR for Cytokines. Expression of IL-8, IL-6 and TNF-α was determined by RT-PCR as reported by Baranova, I. et al. (2002) (“LIPOPOLYSACCHARIDE DOWN REGULATES BOTH SCAVENGER RECEPTOR B1 AND ATP BINDING CASSETTE TRANSPORTER A1 IN RAW CELLS,” Infect. Immun. 70:2995-3003). Glyceraldehyde-3-phosphate dehydrogenase (GAPDH) is used as a reference. Forward and reverse primers are shown in Table 2.

TABLE 2

Forward and Reverse Primers used in RT-PCR

| Primer | SEQ ID | | |
|---|---|---|---|
| # | NO: | Protein | Sequence |
| 1 | 15 | TNF-α | (F) TCCAGGACCTTACCA |
| | 16 | | (R) GGATGAAGCAGCAGC |
| 2 | 17 | IL-6 | (F) AGCGCCTTGGGTCCAGTTGC |
| | 18 | | (R) GCAGCTTCGTCAGCAGGCTG |
| 3 | 19 | IL-8 | (F) AAGCTGGCCGTGGCTCTCTTG |
| | 20 | | (R) CATAATTTCTGTGTTGGCGCAGTG |
| 4 | 1 | GAPDH | (F) GTCTTCACCACCATGGAGAAG |
| | 2 | | (R) GCTTCACCACCTTCTTGATGTCATC |

Competition Experiments. The cells are incubated in 1 μg/ml Bodipy-LPS in the presence increasing concentrations of studied peptides. After 2-h incubation cells are washed with ice-cold PBS and lysed in 0.1% sodium dodecyl sulfate (SDS). The lysate fluorescence is measured by HTS7000 Bioassay reader (Perkin Elmer) using 488 nm for excitation and 533 nm for emission monitoring.

### Results

CLA-1 Overexpression Increases L-37PA, ApoA-I, HDL, and Monomeric LPS Uptake in HeLa Cells. The effect of Cla-1 expression on the cell binding and internalization of Alexa 568-L-37PA, Alexa 568-apoA-I, Alexa 488-HDL, and Bodipy LPS is assessed by confocal scanning laser microscopy, using stably transfected CLA-1 expressing HeLa cells (Vishnyakova, T. G. et al. (2003) “BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780). Increased surface binding and internalization is observed for all four ligands. A large accumulation of Bodipy-LPS and L-37PA is found in the perinuclear compartment, which has been identified as a primary site for LPS accumulation in mononuclear cells (Thieblemont, N. et al. (1999) “TRANSPORT OF BACTERIAL LIPOPOLYSACCHARIDE TO THE GOLGI APPARATUS,” J. Exp. Med. 190:523-534). Additionally, the uptake of the single amphipathic helix 18A and L2D-37PA containing two-amino acid substitutions in each helix, which disturb the helix structure without significantly affecting of the overall charge of the peptide, is not increased in CLA-1 transfected HeLa cells when compared with a mock-transfected control.

L-37PA, apoA-I, and Monomeric LPS Co-localize with Golgi Apparatus Markers. In contrast to LPS aggregates, monomeric LPS is known to be transported to the Golgi complex (Thieblemont, N. et al. (1999) “TRANSPORT OF BACTERIAL LIPOPOLYSACCHARIDE TO THE GOLGI APPARATUS,” J. Exp. Med. 190:523-534). Confocal microscopy demonstrates that the staining pattern was similar for Bodipy-LPS, Alexa568-ApoA-I and Alexa568-L-37PA. Extensive overlap is observed for fluorescent ceramide and fluorescent SRBI ligands in the perinuclear compartment, thus confirming a predominant accumulation of LPS, apoA-I and L-37PA in the Golgi apparatus. These results are confirmed using anti-COP1 antibody, another trans-Golgi marker. Much less extensive overlap is observed for fluorescent transferrin and L-37PA (LPS) indicating that the endocytic recycling compartment was a secondary transporting site for CLA-1 ligands.

Co-localization of LPS, L-37PA and ApoA-I in CLA-1 Overexpressing HeLa Cells. To determine whether LPS is internalized and transported to the same compartment(s) as classical SR-BI/CLA-1 ligands such as apoA-I and class A helical amphipathic peptides, uptake and colocalization of Bodipy-LPS with labeled SRBI ligands are analyzed. Co-incubation of Bodipy-LPS and Alexa 568-apoA-I for 1 h at 37° C. lead to extensive ligand binding and internalization, predominantly co-localizing in the perinuclear compartment. Intracellular LPS and apoA-I appear in a characteristic punctuate pattern, as reported by Vishnyakova, T. G. et al. (2003) (“BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780). Both surface bound, intracellular and perinuclear Bodipy-LPS extensively overlap with Alexa-568-ApoA-I, indicating that both ligands are transported utilizing a similar intracellular pathway to the intracellular compartment, which has been determined to be the Golgi complex. The L-37PA peptide closely mimics the functional activity of apoA-I including the ability to activate acyl CoA: cholesterol acyltransferase (ACAT), mediate cholesterol efflux, bind lipid, and serve as a potent SR-BI/CLA-1 ligand. Co-incubation of Bodipy-LPS and Alexa 568-L-37PA is associated with intensive accumulations of both labels on the plasma membrane and in the perinuclear area of HeLa cells. Extensive areas of co-localization appear in both locations, suggesting a similar transporting pathway for these labels. These results are consistent with the Golgi apparatus being the primary delivery site of SR-BI/CLA-1 ligands through endocytic vesicular transport. Similar results are observed in phorbol myristate acetate (PMA)-differentiated THP-1 cells.

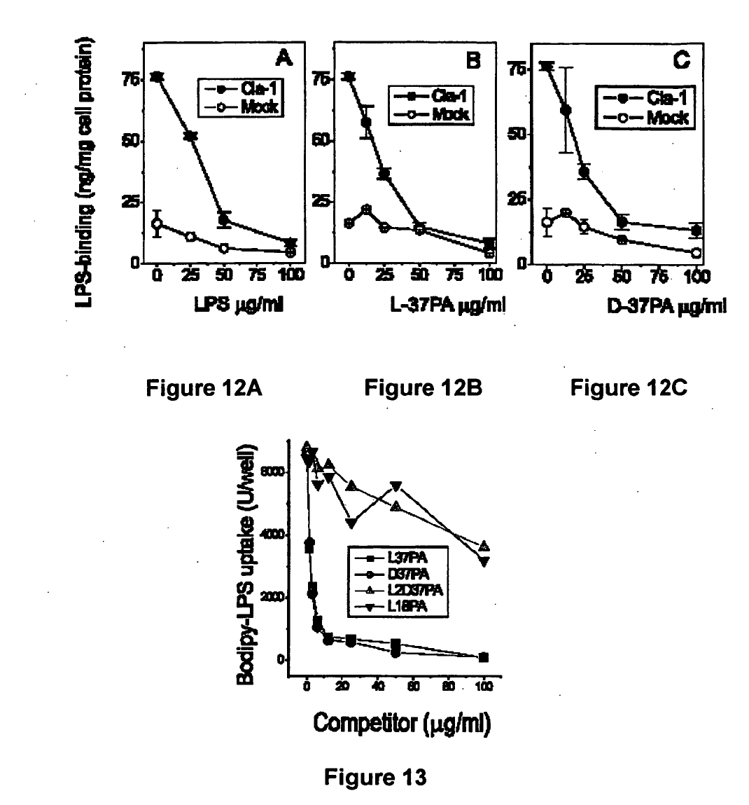

L-37PA and D-37PA Compete Against LPS in Control and CLA-1 Overexpressing HeLa Cells. Competition experiments seen in FIGS. 12A, 12B and 12C demonstrate that ¹²⁵I-LPS binding is increased in CLA-1 expressing HeLa cells. LPS, L-37PA and D-37PA, a class A peptide with the same sequence as L-37PA synthesized with D-amino acids, competed in a dose-dependent manner against 125I-LPS in both mock-transfected and CLA-1 expressing cells. Incubation of CLA-1 overexpressing cells with Bodipy-LPS in the presence of 100-fold L-37PA excess markedly decreases surface binding of LPS and intracellular LPS transport to the Golgi complex. In contrast, co-incubation of Bodipy-LPS in the presence of 100-fold L2D-37PA excess, which contains two D amino acid substitutions and does not form helices, does not decrease LPS uptake (FIG. 13).

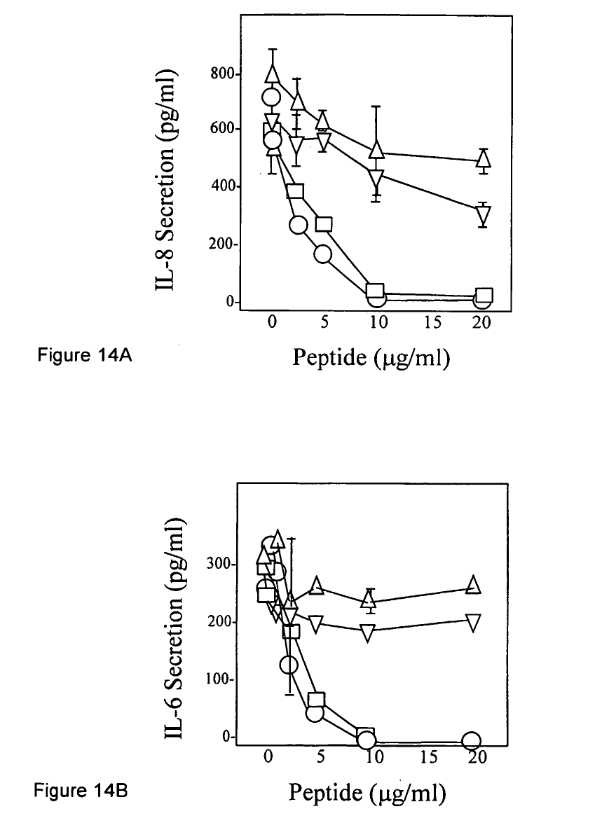

L-37PA and D-37PA Block LPS-induced Cytokine Production in THP-1 Cells. Since HeLa cells typically do not demonstrate LPS induced cytokine secretion (Vishnyakova, T. G. et al. (2003) “BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780), the THP-1 human monocyte cell line, which has been used extensively as a LPS-responsive model is employed. As seen in FIGS. 14A and 14B, L-37PA dose-dependently blocks LPS-stimulated secretion of IL-8, IL-6 and TNF-α. D-37PA demonstrates a similar blocking effect. Decreased mRNA levels for IL-8 and TNF-α as determined by RT-PCR are also observed. Neither L2D-37PA nor 18A, a single helix containing peptide, are effective inhibitors of LPS-stimulated cytokine secretion.

L-37PA Does Not Affect LPS Activity By The LAL Test. A number of amphipathic helical peptides are thought to form complexes with endotoxin, neutralizing LPS (Hirata, M. et al. (1995) “STRUCTURE AND FUNCTIONS OF ENDOTOXIN-BINDING PEPTIDES DERIVED FROM CAP18,” Prog. Clin. Biol Res. 392:317-326; Garber, D. W. et al. (2001) “A NEW SYNTHETIC CLASS A AMPHIPATHIC PEPTIDE ANALOGUE PROTECTS MICE FROM DIET-INDUCED ATHEROSCLEROSIS,” J. Lipid Res. 42:545-552; Nagaoka, I. et al. (2001) “CATHELICIDIN FAMILY OF ANTIBACTERIAL PEPTIDES CAP 18 AND CAP11 INHIBIT THE EXPRESSION OF TNF-ALPHA BY BLOCKING THE BINDING OF LPS TO CD14(+) CELLS,” J. Immunol. 167:3329-3338). To examine if the peptide blockage of LPS activity reflects the formation of an inactive complex, 10 ng/ml of LPS is incubated with 10 μg/ml L-37PA or L2D-37PA in PBS, DPBS (which contains Ca²⁺ and Mg²⁺) as well as in RPMI-1640 containing 1% FCS (the media used in the LPS-induced cytokine secretion experiments) at 37° C. for 24 h. The LPS-activity is measured by a chromogenic Limulus amebocyte lysate assay. No substantial LPS neutralization is observed in RPMI 1640 and DPBS, which contained bivalent cations. In the absence of cations, L-37PA significantly reduces LPS activity as measured by LAL. These findings are consistent with earlier reports that cations decreased the neutralizing activity of plasma components (Zhang, G. H. et al. (1995) “QUANTIFICATION OF THE ENDOTOXIN-NEUTRALIZING CAPACITY OF SERUM AND PLASMA,” APMIS 103:721-730, Ogata, M. et al. (1997) “EFFECT OF ANTICOAGULANTS ON BINDING AND NEUTRALIZATION OF LIPOPOLYSACCHARIDE BY THE PEPTIDE IMMUNOGLOBULIN CONJUGATE CAP18 (106-138)-IMMUNOGLOBULIN G IN WHOLE BLOOD,” Infect. Immun. 65:2160-2167) and indicate that the L-37PA inhibition of LPS-induced cytokine production unlikely resulted from LPS neutralization.

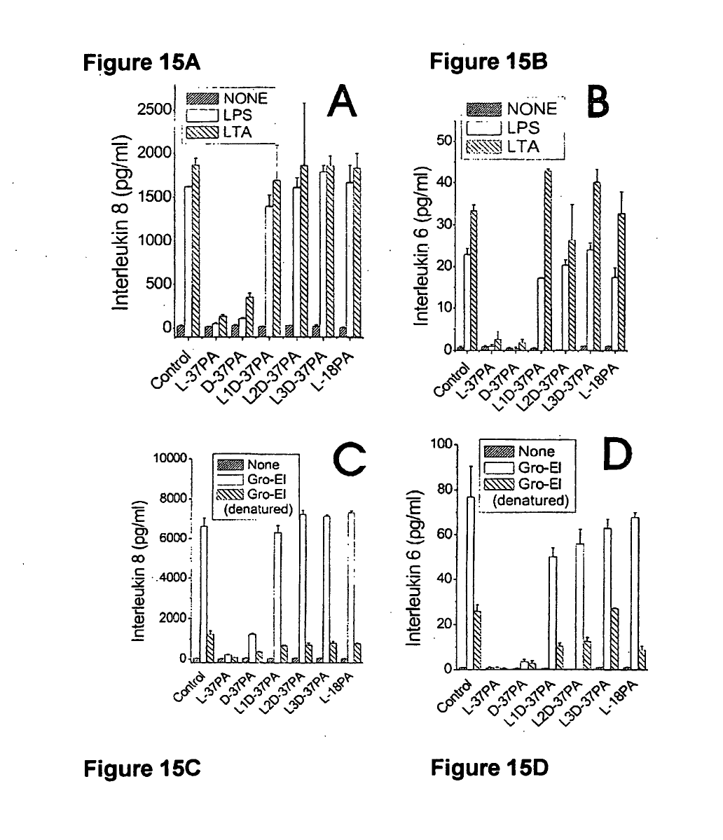

L-37PA Prevents LTA and Gro-EL Stimulated Production of IL-8. It is thought that various bacterial components, including LTA, a cell wall component of gram-positive bacteria, and cytoplasmic bacterial heat shock protein, chaperonin 60 or Gro-EL, elicit their effect by inducing down stream signaling by activating receptors, which belong to the TLR family. To study whether their effects can be blocked by L-37PA treatments, THP-1 cells are incubated with 1 μg/ml LTA or 5 μg/ml Gro-EL in the presence of the L-37PA, 18A or L-37PA peptides with single (L1D-37PA), double (L2D-37PA) or triple (L3D-37PA) D-amino acid substitutions which progressively abolish helical peptide structure. As seen at FIGS. 15A, 15B, 15C and 15D, a 10-200 fold excess of L-37PA blocks production of IL-8 and IL-6 elicited by LTA and Gro-EL. Peptides synthesized with single, double or triple D to L amino acid substitutions are not effective blockers of both LPS and LTA induced IL-8 secretion, indicating that an amphipathic helical structure plays a critical role in the blocking efficiency of the peptides. The low blocking efficiency of 18A indicates a requirement of at least a double helix for blocking.

### Discussion

Bacterial uptake by cellular scavenger receptors and initiation of an inflammatory reaction are important parts of the innate immune system, which protects an organism during the initial contact with an infectious entity. Gene knock-out experiments indicate that mice deficient in the expression of various scavenger receptors, TLR or receptors recognizing bacterial cell wall components such as CD14 exhibit increased sensitivity to bacterial infections (Chow, J. C. et al. (1999) “TOLL-LIKE RECEPTOR-4 MEDIATES LIPOPOLYSACCHARIDE-INDUCED SIGNAL TRANSDUCTION,” J. Biol. Chem. 274:10689-10692; Haworth, R. et al. (1997) “THE MACROPHAGE SCAVENGER RECEPTOR TYPE A IS EXPRESSED BY ACTIVATED MACROPHAGES AND PROTECTS THE HOST AGAINST LETHAL ENDOTOXIC SHOCK,” J. Exp. Med. 186:1431-1439).

Scavenger receptors are a family of cell surface glycoproteins including Class A, B and D (SR-A, SR-B, SR-D), which are able to bind modified lipoproteins and high-density lipoprotein (HDL). This receptor family is characterized by a wide ligand specificity and predominantly reside in phagocytes, hepatocytes and steroid hormone producing cells. Multiple studies have established an important role of class A scavenger receptors in bacterial binding and internalization (Underhill, D. M. et al. (2002) “PHAGOCY-rOSIS OF MICROBES: COMPLEXITY IN ACTION,” Annu. Rev. Immunol. 20:825-852), antigen presentation and cell adhesion (Gordon, S. (1998) “THE ROLE OF THE MACROPHAGE IN IMMUNE REGULATION,” Res. Immunol. 149:685-688), processes involved with host defense during infections. In contrast, a role for the class B scavenger receptor, especially, human orthologue CLA-1, has not been intensively studied for a role in infection and/or inflammation.

The physiologic importance of the interaction of SR-BI/CLA-1 with its ligands, such as HDL (apoA-I), has been established by a variety of in vivo studies, primarily using rodent models. SR-BI/CLA-1 affects the structure and composition of plasma HDL, including the cholesterol and cholesterol ester content of HDL. SRBI/CLA-1 also regulates cholesterol levels in the adrenal gland, ovary, and bile by mediating selective cholesterol ester uptake in these SR-BI/CLA-1 abundantly expressing organs. Recent observations also indicate that SR-BI/CLA-1 expression is regulated by LPS in monocyte cell lines (Baranova, I. et al. (2002) “LIPOPOLYSACCHARIDE DOWN REGULATES BOTH SCAVENGER RECEPTOR B1 AND ATP BINDING CASSETTE TRANSPORTER A1 IN RAW CELLS,” Infect. Immun. 70:2995-3003), and that SR-BI/CLA-1 binds and internalizes LPS (Vishnyakova, T. G. et al. (2003) “BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780). Since overexpression of SR-BI/CLA-1 causes LPS to be transported to the trans-Golgi network, SR-BI/CLA-1 appears to function as an endocytic LPS-receptor. In this Example, CLA-1 overexpressing cells demonstrate an increased L-37PA and Bodipy-LPS uptake and internalization into the Golgi complex. Importantly, both apoA-I and L-37PA are transported to the same cellular compartment. Moreover, when incubated together with LPS, fluorescent signals merge though all cellular compartments, indicating the same transport pathway for all three SR-BI/CLA-1 ligands. The Golgi complex, as well as plasma cholesterol-rich membrane microdomains (rafts), have been reported to be major sites for the TLR family (Triantafilou, M. et al. (2002) “MEDIATORS OF INNATE IMMUNE RECOGNITION OF BACTERIA CONCENTRATE IN LIPID RAFTS AND FACILITATE LIPOPOLYSACCHARIDE-INDUCED CELL ACTIVATION,” J Cell Sci. 115:2603-2611), the receptors involved with direct activation of the IκB/nuclear factor-κB system. Particularly, in the vascular wall, most of the vascular inflammatory responses are mediated through the IκB/nuclear factor-κB system. Vascular inflammation can be limited by anti-inflammatory counteregulatory agents, including HDL and exchangeable apolipoproteins, such as apoA-I (Shah, P. K. et al. (2001) “EXPLOITING THE VASCULAR PROTECTIVE EFFECTS OF HIGH-DENSITY LIPOPROTEIN AND ITS APOLIPOPROTEINS: AN IDEA WHOSE TIME FOR TESTING IS COMING, PART I,” Circulation 104:2376-2383, Cockerill, G. W. et al. (2001) “ELEVATION OF PLASMA HIGH-DENSITY LIPOPROTEIN CONCENTRATION REDUCES INTERLEUKIN-1-INDUCED EXPRESSION OF E-SELECTIN IN AN IN VIVO MODEL OF ACUTE INFLAMMATION,” Circulation 103:108-112). It has been reported that HDL and exchangeable apolipoproteins neutralize LPS proinflammatory activity and prevent LDL oxidation (Van Lenten, B. J. et al. (2001) “HIGH-DENSITY LIPOPROTEIN LOSES ITS ANTI-INFLAMMATORY PROPERTIES DURING ACUTE INFLUENZA A INFECTION,” Circulation 103:2283-2288). In addition, HDL and exchangeable apolipoproteins also inhibit the production of IL-1β and TNF-α by blocking contact-mediated activation of monocytes by T lymphocytes (Hyka, N. et al. (2001) “APOLIPOPROTEIN A-I INHIBITS THE PRODUCTION OF INTERLEUKIN-1 BETA AND TUMOR NECROSIS FACTOR-ALPHA BY BLOCKING CONTACT-MEDIATED ACTIVATION OF MONOCYTES BY T LYMPHOCYTES,” Blood 97:2381-2389) and block cytokine-induced expression of E-selectin, ICAM-1, and VCAM-1 on endothelial cells at the transcriptional level (Cockerill, G. W. et al. (2001) “ELEVATION OF PLASMA HIGH-DENSITY LIPOPROTEIN CONCENTRATION REDUCES INTERLEUKIN-1-INDUCED EXPRESSION OF E-SELECTIN IN AN IN VIVO MODEL OF ACUTE INFLAMMATION,” Circulation 103:108-112, Moudry, R. et al. (1997) “RECONSTITUTED HIGH DENSITY LIPOPROTEIN MODULATES ADHERENCE OF POLYMORPHONUCLEAR LEUKOCYTES TO HUMAN ENDOTHELIAL CELLS,” Shock 7:175-181).

Class A amphipathic helical peptides have also been demonstrated to mimic the anti-inflammatory properties of HDL in vivo and in vitro (Garber, D. W. et al. (2001) “A NEW SYNTHETIC CLASS A AMPHIPATHIC PEPTIDE ANALOGUE PROTECTS MICE FROM DIET-INDUCED ATHEROSCLEROSIS,” J. Lipid Res. 42:545-552). When injected into an animal as a phospholipid vesicle, one of the analogues prevented endotoxic shock induced by LPS (Levine, D. M. et al. (1993) “IN VIVO PROTECTION AGAINST ENDOTOXIN BY PLASMA HIGH DENSITY LIPOPROTEIN,” Proc. Natl. Acad. Sci. U.S.A 90:12040-12044). A class A amphipathic helical peptide has also has been reported to protect mice from diet-induced atherosclerosis (Garber, D. W. et al. (2001) “A NEW SYNTHETIC CLASS A AMPHIPATHIC PEPTIDE ANALOGUE PROTECTS MICE FROM DIET-INDUCED ATHEROSCLEROSIS,” J. Lipid Res. 42:545-552), as well as dramatically reducing atherosclerosis in LDL-receptor deficient mice independently of the plasma cholesterol level (Navab, M. et al. (2002) “ORAL ADMINISTRATION OF AN APO A-I MIMEIC PEPTIDE SYNTHESIZED FROM D-AMINO ACIDS DRAMATICALLY REDUCES ATHEROSCLEROSIS IN MICE INDEPENDENT OF PLASMA CHOLESTEROL,” Circulation 105, 290-292). It has been recently demonstrated that another amphipathic peptide mimicking apoA-I, D-4F, prevented a marked increase in activated macrophage traffic into the aortic arch induced by an influenza viral infection (Van Lenten, B. J. et al. “INFLUENZA INFECTION PROMOTES MACROPHAGE TRAFFIC INTO ARTERIES OF MICE THAT IS PREVENTED BY D-4F, AN APOLIPOPROTEIN A-I MIMETIC PEPTIDE,” (2002) Circulation 106, 1127-1132). The mechanisms responsible for such anti-inflammatory properties of peptides are unknown, however, the demonstration that human SR-BI/CLA-1 (and its human homolog CLA-1) is an endocytic LPS-receptor suggests that apoA-I mimicking peptides, which are also SR-BI/CLA-1 ligands Williams, D. L. et al. (2000) “BINDING AND CROSS-LINKING STUDIES SHOW THAT SCAVENGER RECEPTOR BI INTERACTS WITH MULTIPLE SITES IN APOLIPOPROTEIN A-I AND IDENTIFY THE CLASS A AMPHIPATHIC ALPHA-HELIX AS A RECOGNITION MOTIF,” J. Biol. Chem 275:18897-18904 (25), can elucidate their anti-inflammatory effect by targeting SR-BI/CLA-1 (Vishnyakova, T. G. et al. (2003) “BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780). A direct competition of L-37PA and D-37PA with LPS in CLA-1 overexpressing HeLa cells indicates that peptides can elicit their anti-inflammatory effect by competing with proinflammatory compounds, such as LPS. When the effect of the peptides is studied in THP-1 cells, a marked decrease of LPS-stimulated cytokine expression is observed. Importantly, 18A, a less potent SR-BI/CLA-1 ligand as well as L2D-37PA, which does not contain a helical structure and do not bind to SR-BI/CLA-1, are without effect.

A number of amphipathic helical peptides based on anti-bacterial proteins have been reported to protect animals against endotoxic shock by a forming a complex and neutralizing LPS (Hirata, M. et al. (1995) “STRUCTURE AND FUNCTIONS OF ENDOTOXIN-BINDING PEPTIDES DERIVED FROM CAP18,” Prog. Clin. Biol Res. 392:317-326; Nagaoka, I. et al. (2001) “CATHELICIDIN FAMILY OF ANTIBACTERIAL PEPTIDES CAP18 AND CAP11 INHIBIT THE EXPRESSION OF TNF-ALPHA BY BLOCKING THE BINDING OF LPS TO CD14(+) CELLS,” J. Immunol. 167:3329-3338). To exclude the possibility that neutralization of LPS by L-37PA is a major factor, the effect of LPS/L-37PA incubation on LPS activity is studied using the LAL test. The outcome of this experiment indicates that L-37PA does not neutralize LPS when incubated in the same calcium containing media used for cytokine detection experiments in THP-1 cells (RPMI-1640 containing 1% FCS). In the absence of bivalent cations (PBS containing 1% serum, not DPBS which contains bivalent cations) L-37PA bound LPS and decreased LPS activity in the LAL test. These data are in an agreement with the general observation that bivalent cations dramatically reduce LPS neutralization by plasma components (Zhang, G. H. et al. (1995) “QUANTIFICATION OF THE ENDOTOXIN-NEUTRALIZING CAPACITY OF SERUM AND PLASMA,” APMIS 103:721-730, Ogata, M. et al. (1997) “EFFECT OF ANTICOAGULANTS ON BINDING AND NEUTRALIZATION OF LIPOPOLYSACCHARIDE BY THE PEPTIDE IMMUNOGLOBULIN CONJUGATE CAP18 (106-138)-IMMUNOGLOBULIN G IN WHOLE BLOOD,” Infect. Immun. 65:2160-2167). Interesting, L2D-37PA, a peptide lacking helical structure, also decreases LPS activity in the absence of bivalent cations and had no effect in the presence of bivalent cations. Since L2D-37PA appears to block neither LPS uptake nor LPS/LTA/Gro-EL induced interleukin (IL-6 and IL-8) secretion while was a relatively effective LPS-neutralizer, it appears that the neutralization effect seen in the absence of calcium is likely to result from a lipid binding activity of the peptides (Remaley, A. T. et al. “SYNTHETIC AMPHIPATHIC HELICAL PEPTIDES PROMOTE LIPID EFFLUX FROM CELLS BY AN ABCA1-DEPENDENT AND AN ABCA1-INDEPENDENT PATHWAY,” (2003) J. Lipid Res. 44, 828-836). To determine if L-37PA is a toxic peptide, the effect of the peptide on the TNFα-induced IL-6 as well as IL-8 secretion was measured in THP-1 cells. Neither TNFα-induced IL-6 nor IL-8 secretion, LDH release nor of β-actin expression are affected by incubation with 20 μg/ml L-37PA, indicating intact inflammatory pathways and no apparent toxic effect in THP-1 cells. These results contrast with anti-bacterial helical cathelicin-derived peptides, which being highly positively charged and bactericidal (Travis, S. M. (2000) “BACTERICIDAL ACTIVITY OF MAMMALIAN CATHELICIDIN-DERIVED PEPTIDES,” Infect. Immun. 68:2748-2755), are toxic to mammalian cells at high concentrations (Risso, A. et al. (1998) “CYTOTOXICITY AND APOPTOSIS MEDIATED BY TWO PEPTIDES OF INNATE IMMUNITY,” Gel/Immunol. 189:107-115).

This study also demonstrates that L-37PA blocks the proinflammatory response induced by LTA, an amphipathic membrane component of gram positive bacteria. In contrast to L-37PA, no effect was observed for non-helical peptides L1D-37PA, L2D-37PA or L3D-37PA. Importantly, the peptides made with a mixture of L and D amino acids had lower lipid affinity, as assessed by monitoring their ability to act as detergents in the solubilization of DMPC vesicles in the order L-37PA>L1D-37PA>L2D37PA>L3D-37PA. However, a similar ability to stimulate cholesterol efflux was demonstrated in HeLa cells with these different peptides (Remaley, A. T. et al. “SYNTHETIC AMPHIPATHIC HELICAL PEPTIDES PROMOTE LIPID EFFLUX FROM CELLS BY AN ABCA1-DEPENDENT AND AN ABCA1-INDEPENDENT PATHWAY,” (2003) J. Lipid Res. 44, 828-836). Since the L-D substituted peptides are neither CLA-1 ligands nor effective anti-inflammatory blockers, it appears that non-selective cholesterol depletion was not a factor in L-37PA related blockage of LPS/LTA/Gro-EL-stimulated cytokine production. The amphipathic properties of LTA suggest that SR-BI/CLA-1 can act as an endocytic LTA-receptor. Amphipathic helical ligands also blocked proinflammatory responses induced by Gro-EL, cytoplasmic bacterial chaperon 60. It is likely that other proinflammatory bacterial and animal compounds may also use this receptor since IL-6 and IL-8 secretion induced by human HSP60, a highly helical proinflammatory molecule (Chen, W. et al. (1999) “HUMAN 60-KDA HEAT-SHOCK PROTEIN: A DANGER SIGNAL TO THE INNATE IMMUNE SYSTEM,” J. Immunol. 162:3212-3219), was also blocked by L-37PA. The data thus support the conclusion that in addition to its well-established role in HDL metabolism (Babitt, J. et al. (1997) “MURINE SR-BI, A HIGH DENSITY LIPOPROTEIN RECEPTOR THAT MEDIATES SELECTIVE LIPID UPTAKE, IS N-GLYCOSYLATED AND FATTY ACYLATED AND COLOCALIZES WITH PLASMA MEMBRANE CAVEOLAE,” J. Biol. Chem. 272:13242-13249) and HDL-related signaling, SR-BI/CLA-1 plays an important role in the intracellular trafficking of various bacterial and mammalian proinflammatory components and could also participate in their signaling.

In summary, the data presented herein demonstrate that SR-BI/CLA-1 targeting by synthetic amphipathic helical peptides block LPS as well as LTA and Gro-EL-induced proinflammatory responses in cells. The effect on LPS appears to result from a competition of the L-37PA with LPS for the LPS-endocytic receptor, CLA-1. The data indicate that SR-BI/CLA-1 targeting by L-37PA eliminates LPS binding to the plasma membrane and transport to the Golgi complex, two major sites of TLR receptor localization. These data provide important insights into the mechanisms of the anti-inflammatory and anti-infection properties seen with plasma high density lipoproteins and exchangeable apolipoproteins. Since the effects of various bacterial compounds were blocked by CLA-1 ligands, the amphipathic helical motif of exchangeable apolipoproteins may represent a general host defense mechanism against inflammatory reactions. Additionally, agents targeting CLA-1 may represent a new class of therapeutics for infections and inflammation.

### Example 5

### Human Scavenger Receptor Class B Type I, CLA-1, Functions as an Endocytic Serum Amyloid A Receptor, Leading to Partial SAA Degradation

The serum amyloid A (SAA) family of proteins is encoded by multiple genes, which display allelic variation and a high degree of homology in mammals (Uhlar, C. M. & Whitehead, A. S. (1999) “SERUM AMYLOID A, THE MAJOR VERTEBRATE ACUTE-PHASE REACTANT,” Eur. J. Biochem. 265:501-523). Acute phase SAA (SAA1 and SAA2) are 12-14 kDa proteins whose concentrations are increased by 1000-fold in response to inflammation or extensive tissue damage (Uhlar, C. M. & Whitehead, A. S. (1999) “SERUM AMYLOID A, THE MAJOR VERTEBRATE ACUTE-PHASE REACTANT,” Eur. J. Biochem. 265:501-523). SAA is conserved among vertebrates, and is involved with inflammatory responses (Uhlar, C. M. & Whitehead, A. S. (1999) “SERUM AMYLOID A, THE MAJOR VERTEBRATE ACUTE-PHASE REACTANT,” Eur. J. Biochem. 265:501-523), chemotaxis (Kumon, Y. et al. (2002) “ACUTE-PHASE, BUT NOT CONSTITUTIVE SERUM AMYLOID A (SAA) IS CHEMOTACTIC FOR CULTURED HUMAN AORTIC SMOOTH MUSCLE CELLS,” Amyloid. 9:237-241), and pathogenic amyloid A fibril deposition in amyloidosis (Uhlar, C. M. & Whitehead, A. S. (1999) “SERUM AMYLOID A, THE MAJOR VERTEBRATE ACUTE-PHASE REACTANT,” Eur. J. Biochem. 265:501-523). Acute phase SAA (SAA1 and SAA2).

Liver is a major source of acute-phase SAA production during inflammation. However extrahepatic SAA expression has also been documented in smooth muscle cells, endothelial cells and macrophages (Ramadori, G. et al. (1985) “EXPRESSION AND REGULATION OF THE MURINE SERUM AMYLOID A (SAA) GENE IN EXTRAHEPATIC SITES,” J. Immunol. 135:3645-3647). Inflammatory cytokines such as IL-1β, TNF-α and IL-6 are potent inducers of SAA synthesis in the liver and peripheral organs containing large numbers of reticular-endothelial cells (Uhlar, C. M. & Whitehead, A. S. (1999) “SERUM AMYLOID A, THE MAJOR VERTEBRATE ACUTE-PHASE REACTANT,” Eur. J. Biochem. 265:501-523). In the circulation, at lower levels, SAA is predominantly associated with HDL while at higher SAA concentrations there is dissociation from HDL (Bausserman, L. L. et al. (1984) “RAPID CLEARANCE OF SERUM AMYLOID A FROM HIGH-DENSITY LIPOPROTEINS,” Biochim. Biophys. Acta 792:186-191; Coetzee, G. A. et al. (1986) “SERUM AMYLOID A-CONTAINING HUMAN HIGH DENSITY LIPOPROTEIN 3. DENSITY, SIZE, AND APOLIPOPROTEIN COMPOSITION,” J. Biol. Chem. 261:9644-9651). Free SAA is also found in inflammatory sites (Kumon, Y. et al. (1999) “LOCAL EXPRESSION OF ACUTE PHASE SERUM AMYLOID A MRNA IN RHEUMATOID ARTHRITIS SYNOVIAL TISSUE AND CELLS,” J. Rheumatol. 26:785-790; Meek, R. L. et al. (1994) “EXPRESSION OF APOLIPOPROTEIN SERUM AMYLOID A MRNA IN HUMAN ATHEROSCLEROTIC LESIONS AND CULTURED VASCULAR CELLS: IMPLICATIONS FOR SERUM AMYLOID A FUNCTION,” Proc. Natl. Acad. Sci. U.S. A 91: 3186-3190), suggesting a role of SAA in local inflammation.

Despite SAA's functional role being incompletely understood, a number of studies have demonstrated that SAA is a potent inducer of cholesterol efflux and contains at least two lipid binding domains (Kisilevsky, R. et al. (2002) “NOVEL GLYCOSAMINOGLYCAN PRECURSORS AS ANTI-AMYLOID AGENTS, PART III,” Pediatr. Pathol. Mol. Med. 21: 291-305). It is likely that lipid free SAA induces both ABCA-1 cassette transporter-dependent and energy-independent, passive cholesterol efflux (Kisilevsky, R. et al. (2003) “MACROPHAGE CHOLESTEROL EFFLUX AND THE ACTIVE DOMAINS OF SERUM AMYLOID A2.1,” J. Lipid Res. (e-Publ)). The later observation suggests that lipid-free SAA may participate in cholesterol and lipid resorption in a necrotic area, while lipoprotein associated SAA, which increases lipoprotein binding and selective HDL cholesterol ester uptake, may facilitate lipid uptake and eventual metabolism in the liver (Artl, A. et al. (2000) “ROLE OF SERUM AMYLOID A DURING METABOLISM OF ACUTE-PHASE HDL BY MACROPHAGES,” Arterioscler. Thromb. Vasc. Biol. 20:763-772). In addition, SAA may play an immune-related role in a number of pathogenic conditions. Lipid-free SAA is a strong inducer of IL-8 production in neutrophils (He, R. et al. (2003) “SERUM AMYLOID A INDUCES IL-8 SECRETION THROUGH A G PROTEIN-COUPLED RECEPTOR, FPRL1/LXA4R,” Blood 101:1572-1581) as well as IL-1β in THP-1 cells (Hayat, S. et al. (2000) “ACUTE PHASE SERUM AMYLOID A PROTEIN INCREASES HIGH DENSITY LIPOPROTEIN BINDING TO HUMAN PERIPHERAL BLOOD MONONUCLEAR CELLS AND AN ENDOTHELIAL CELL LINE,” Scand. J. Immunol. 51:141-146). SAA has been demonstrated to bind to formyl peptide seven-transmembrane-spanning Gprotein-coupled receptors (FPR and FPR1) involved with Ca2+ mobilization and protein kinase C activation (He, R. et al. (2003) “SERUM AMYLOID A INDUCES IL-8 SECRETION THROUGH A G PROTEIN-COUPLED RECEPTOR, FPRL1/LXA4R,” Blood 101:1572-1581). Overexpression of FPR1, but not FPR, in human embryonic kidney cells confers both SAA-induced pertussis-toxin-sensitive Ca²⁺ mobilization and cell migration (He, R. et al. (2003) “SERUM AMYLOID A INDUCES IL-8 SECRETION THROUGH A G PROTEIN-COUPLED RECEPTOR, FPRL1/LXA4R,” Blood 101:1572-1581). This pathway is likely associated with a recruitment of inflammatory cells to sites of inflammation, an important step in innate and adaptive immune responses (Badolato, R. et al. (2000) “SERUM AMYLOID A IS AN ACTIVATOR OF PMN ANTIMICROBIAL FUNCTIONS: INDUCTION OF DEGRANULATION, PHAGOCYTOSIS, AND ENHANCEMENT OF ANTI-CANDIDA ACTIVITY,” J. Leukoc. Biol. 67:381-386; Badolato, R. et al. (1995) “SERUM AMYLOID A INDUCES CALCIUM MOBILIZATION AND CHEMOTAXIS OF HUMAN MONOCYTES BY ACTIVATING A PERTUSSIS TOXIN-SENSITIVE SIGNALING PATHWAY,” J. Immunol. 155:4004-4010).

It has been demonstrated that macrophages are the primary sites of SAA uptake, metabolism and degradation (Takahashi, M. et al. (1989) “ULTRASTRUCTURAL EVIDENCE FOR INTRACELLULAR FORMATION OF AMYLOID FIBRILS IN MACROPHAGES,” Virchows Arch. A Pathol. Anat. Histopathol. 415:411-419; Kluve-Beckerman, B. et al. (2002) “A PULSE-CHASE STUDY TRACKING THE CONVERSION OF MACROPHAGE-ENDOCYTOSED SERUM AMYLOID A INTO EXTRACELLULAR AMYLOID,” Arthritis Rheum. 46:1905-1913). Amyloid A deposits have been reported both intracellularly and in the direct proximity of macrophages within organs demonstrating amyloid deposits (Kluve-Beckerman, B. et al. (2002) “A PULSE-CHASE STUDY TRACKING THE CONVERSION OF MACROPHAGE-ENDOCYTOSED SERUM AMYLOID A INTO EXTRACELLULAR AMYLOID,” Arthritis Rheum. 46:1905-1913; Elliott-Bryant, R. et al. (1998) “CATABOLISM OF LIPID-FREE RECOMBINANT APOLIPOPROTEIN SERUM AMYLOID A BY MOUSE MACROPHAGES IN VITRO RESULTS IN REMOVAL OF THE AMYLOID FIBRIL-FORMING AMINO TERMINUS,” Scand. J. Immunol. 48:241-247). Cultured macrophages rapidly degrade SAA by a mechanism apparently involving clathrin-dependent trafficking to lysosomes (Kluve-Beckerman, B. et al. (2001) “BINDING, TRAFFICKING AND ACCUMULATION OF SERUM AMYLOID A IN PERITONEAL MACROPHAGES,” Scand. J. Immunol. 53:393-400). Incomplete SAA degradation followed by a recycling of N-terminal, predominantly 76 amino acid-long SAA peptides to the macrophage plasma membrane has been suggested as a major factor in SAA accumulation and Amyloid A fibril deposition in amyloidosis (Elliott-Bryant, R. et al. (1998) “CATABOLISM OF LIPID-FREE RECOMBINANT APOLIPOPROTEIN SERUM AMYLOID A BY MOUSE MACROPHAGES IN VITRO RESULTS IN REMOVAL OF THE AMYLOID FIBRIL-FORMING AMINO TERMINUS,” Scand. J. Immunol. 48:241-247). However a receptor involved with SAA uptake, trafficking and partial degradation has not been reported.

Human scavenger receptor class B type I (CLA-1), an HDL receptor, is highly expressed in macrophages (Baranova, I. et al. (2002) “LIPOPOLYSACCHARIDE DOWN REGULATES BOTH SCAVENGER RECEPTOR B1 AND ATP BINDING CASSETTE TRANSPORTER A1 IN RAW CELLS,” Infect. Immun. 70:2995-3003, Buechler, C. et al. (1999) “LIPOPOLYSACCHARIDE INHIBITS THE EXPRESSION OF THE SCAVENGER RECEPTOR CLA-L IN HUMAN MONOCYTES AND MACROPHAGES,” Biochem. Biophys. Res. Commun. 262:251-254), the major cell population involved with SAA metabolism and SAA-related G-protein linked signaling. Monocyte differentiation into macrophages has been demonstrated to increase CLA-1 expression (Buechler, C. et al. (1999) “LIPOPOLYSACCHARIDE INHIBITS THE EXPRESSION OF THE SCAVENGER RECEPTOR CLA-L IN HUMAN MONOCYTES AND MACROPHAGES,” Biochem. Biophys. Res. Commun. 262:251-254). Structural features of the SAA molecule suggest the involvement of CLA-1 with SAA binding, internalization and possibly signaling events. Two α-helixes corresponding to the 1-18 N-terminal and 72-86 C-terminal sequences are predicted from the SAA sequence (Uhlar, C. M. & Whitehead, A. S. (1999) “SERUM AMYLOID A, THE MAJOR VERTEBRATE ACUTE-PHASE REACTANT,” Eur. J. Biochem. 265:501-523). The presence of such helices is a common feature of exchangeable apolipoproteins, where the Class-A helix represents the major binding motif for CLA-1 binding (Williams, D. L. et al. (2000) “BINDING AND CROSS-LINKING STUDIES SHOW THAT SCAVENGER RECEPTOR BI INTERACTS WITH MULTIPLE SITES IN APOLIPOPROTEIN A-I AND IDENTIFY THE CLASS A AMPHIPATHIC ALPHA-HELIX AS A RECOGNITION MOTIF,” J. Biol. Chem 275:18897-18904; Liu, T. et al. (2002) “THE EFFECTS OF MUTATIONS IN HELICES 4 AND 6 OF APOA-I ON SCAVENGER RECEPTOR CLASS B TYPE I (SR-BI)-MEDIATED CHOLESTEROL EFFLUX SUGGEST THAT FORMATION OF A PRODUCTIVE COMPLEX BETWEEN RECONSTITUTED HIGH DENSITY LIPOPROTEIN AND SR-BI IS REQUIRED FOR EFFICIENT LIPID TRANSPORT,” J. Biol. Chem. 277:21576-21584). Based on this knowledge, it is hypothesized that SR-BI/CLA-1 may function as a SAA-binding protein involved with SAA uptake, trafficking and metabolism. Using the CLA-1 overexpressing cell model, the following data demonstrate that lipid-free SAA binds and is internalized by CLA-1 and that CLA-1 ligands efficiently compete with SAA for CLA-1 binding. CLA-1 related SAA endocytosis leads to partial SAA degradation as demonstrated by the appearance of short length SAA peptides.

### Materials and Methods

Serum Amyloid A1 was obtained from StressGene, CA. Rabbit anti-SR-BI/CLA-1 antibody cross-reacting with the human homologue CLA-1, was from Novus Biological. KKB-1 anti-CLA-1 antibody was used for SAA blocking experiments (Gu, X. et al. (2000) “SCAVENGER RECEPTOR CLASS B, TYPE I-MEDIATED [3H]CHOLESTEROL EFFLUX To HIGH AND Low DENSITY LIPOPROTEINS IS DEPENDENT ON LIPOPROTEIN BINDING TO THE RECEPTOR,” J. Biol. Chem. 275:29993-30001).

Synthesis of amphipathic helical peptides. The peptides are synthesized by a solid-phase procedure as reported by Merrifield, R B. (1969) (“SOLID-PHASE PEPTIDE SYNTHESIS,” Adv. Enzymol. Relat Areas Mol. Biol. 32:221-296) and Fairwell, T. et al. (1987) (“HUMAN PLASMA APOLIPOPROTEIN C-Ii: TOTAL SOLID-PHASE SYNTHESIS AND CHEMICAL AND BIOLOGICAL CHARACTERIZATION,” Proc. Natl. Acad. Sci. U.S. A 84:4796-4800). The sequences of peptides used in the study are shown in Table 1. The amino acid positions where L-amino acids were substituted with D-amino acids are underlined.

Limulus amebocyte lysate (LAL) assay for LPS. The LAL activity of LPS incubated with various peptides is quantitatively determined by a chromogenic Limulus amebocyte lysate test (Kinetic-QCL, BioWhittaker, Walkersville, Md.). The assay is carried out as recommended by the manufacturer and had an analytical sensitivity of 0.005 EU/ml (˜0.5 μg highly purified LPS/ml).

CLA-1 overexpressing HeLa cells. HeLa (Tet-off) cells (Clontech, Pal Alto, Calif.) overexpressing CLA-1 are generated, selected and cultured as reported by Vishnyakova, T. G. et al. (2003) (“BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780).

Preparation of Alexa-SAA and Alexa-HDL. SAA and HDL are conjugated with Alexa-568/488, SE (Molecular Probes, protein labeling kit) following the kit instructions. The Alexa ligands are analyzed by 10-20% Tricine-SDS peptide gel electrophoresis. Gels are scanned using a Fluorocsan (model A, Hitachi). Alexa-labeled preparations of SAA and HDL apolipoproteins are found in appropriate positions with molecular masses of 28, 18 and 12 kDa for apoA-I, apoA-II and SAA respectively.

Ligand-uptake experiments. Binding experiments are performed at 37° C. using concentrations between 1.25 and 30 μg/ml. All incubations are performed in DMEM containing 2 mg/ml BSA. After a 2-hr-incubation on ice, the cells are rinsed with ice-cold PBS and released by a 30-min incubation in EDTA containing Cell stripper (CellGro, USA). Cells are resuspended and added to an equal volume of 4% paraformaldehyde in PBS. Cell fluorescence is analyzed by FACS analyses.

Competition experiments. Cells are cultured for 24 hr in serum free DMEM before experiments. After chilling on ice, cells are incubated in the presence of 5 μg/ml of Alexa 568-SAA and increasing concentrations of cold ligands (SAA, apoA-I and peptides) for 1 hour in DMEM/BSA. For KKB-1 anti-CLA-1 blocking experiments, cells are pre-incubated with KKB-1 rabbit anti-serum (non-immune rabbit serum as a negative control) at a dilution of 1:10 followed by ligand addition to a final concentration of 5 μg/ml and a 1-hour incubation. Cell fluorescence was analyzed by FACS analyses.

Preparation of Alexa488/Alexa488-SAA labeled HDL complexes. HDL (5 mg) are mixed with Alexa 488-SAA (50 μg) in final volume of 1 ml followed by the addition of 2 ml delipidated human plasma and incubated for 24 hours at 37° C. Alexa 488-SAA labeled HDL complexes were re-isolated by a centrifugation in a NaBr gradient (1.072<d<1.216). After extensive dialysis against Ca2+, Mg2+ free PBS, the complexes are filtered (0.22 μm) and stored in a refrigerator up to 2 weeks. The purity of the complexes as determined by fluorescent scanning of native PAGE and agarose gel electrophoresis is close to 100%.

Sites of Alexa 488/568-SAA transport in CLA-1 overexpressing HeLa cells. For studying the sites of SAA delivery, cells are incubated with 5 μg/ml Alexa 488/568-SAA at 37° C. for 2 hours, then washed and chased at 37° C. for 30 minutes in the presence of Bodipy-transferrin, Bodipy-ceramide BSA complex or Lysotracker. Fluorescence is viewed with a Zeiss 510 laser scanning confocal microscope, using a krypton-argon-Omnichrome laser with excitation wavelengths of 488 and 568 nm for Alexa-488 and Alexa-568 labels, respectively.

SAA degradation. HeLa cells are pulsed by incubations with 10 μg/ml Alexa 488-SAA or Alexa 488-SAA/HDL complex for 6 and 18 hours in DMEM containing 20 mg/ml BSA. A conditioned media are collected and stored at 20° C. After cooling cells on ice, the cells are washed with ice-cold PBS, and protein extracted with 2% Triton 100 in PBS as reported by Baranova, I. et al. (2002) (“LIPOPOLYSACCHARIDE DOWN REGULATES BOTH SCAVENGER RECEPTOR B1 AND ATP BINDING CASSETTE TRANSPORTER A1 IN RAW CELLS,” Infect. Immun. 70:2995-3003). Samples of media and cell 2% Triton 100 extracts were subjected to SDS-Tricine PAGE under reducing conditions. Gels were scanned as written above.

### Results

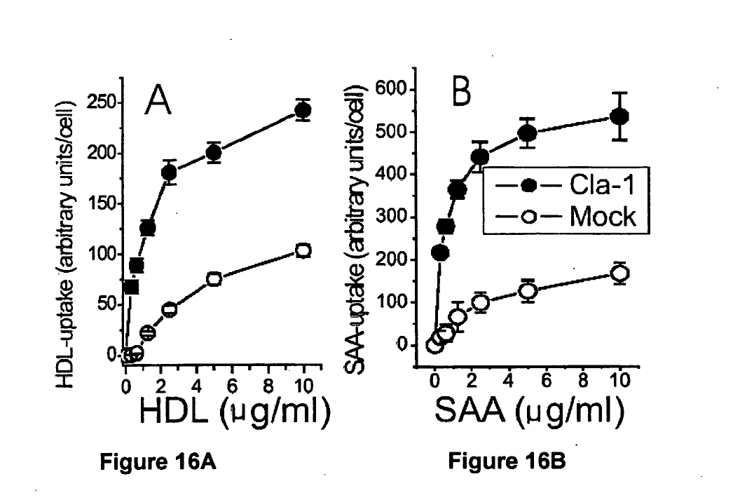

Alexa 488-SAA-uptake in CLA-1 overexpressing HeLa cells. It has been reported that SR-BI/CLA-1 overexpression increases binding and internalization of SR-BI/CLA-1 ligands (Acton, S. L. et al. (1994) “EXPRESSION CLONING OF SR-BI, A CD36-RELATED CLASS B SCAVENGER RECEPTOR,” J. Biol. Chem. 269, 21003-21009; Calvo, D. et al. (1998) “HUMAN CD36 IS A HIGH AFFINITY RECEPTOR FOR THE NATIVE LIPOPROTEINS HDL, LDL, AND VLDL,” J. Lipid Res. 39, 777-788). The functional activity of CLA-1 in overexpressing HeLa cells was demonstrated by observing increased HDL binding and HDL cholesterol ester uptake (Vishnyakova, T. G. et al. (2003) “BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780). To study if SR-BI/CLA-1 also is involved in the uptake of SAA, cells are incubated with increasing concentrations of Alexa 488-SAA and Alexa-488 HDL (control), and analyzed by FACS. As seen on FIG. 16A, CLA-1 overexpressing HeLa cells demonstrate dramatically increased SAA-uptake when compared with a mock-transfected control. SAA-uptake calculated an as arbitrary unit of fluorescence at 488 nm per cell appears to be dose-dependent, plateauing at 2.5 μg/ml SAA (FIG. 16A). Alexa 488-HDL uptake demonstrates a similar dose-dependent accumulation of fluorescent signal in CLA-1 overexpressing HeLa cells, plateauing at 2.5 μg/ml HDL (FIG. 16B).

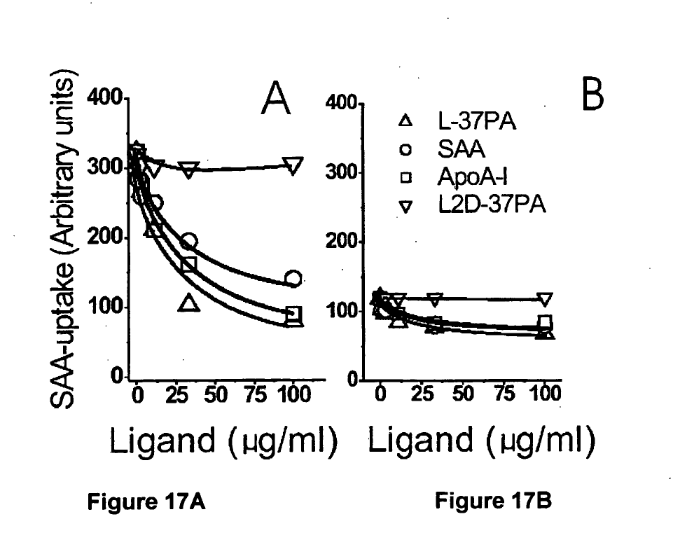

Competition of CLA-1 ligands for SAA-uptake in CLA-1 overexpressing HeLa cells. As seen in FIGS. 17A and 17B, two SR-BI/CLA-1 ligands, lipoprotein-free apoA-I and L-37PA compete with Alexa 488-SAA for CLA-1 in both mock transfected and CLA-1 overexpressing HeLa cells. Cold SAA is also an efficient competitor while the anti-CLA-1 blocking antibody KKB-1 decreased SAA uptake by 70% in both mock-transfected and CLA-1 overexpressing HeLa cells (data not shown). No competition is demonstrated with L1D, L2D and L3D-37PA peptides which contain one, two or three D-amino acid substitutions which disturb the helical structure and ability of the peptides to interact with CLA-1.

SAA and CLA-1 colocalization in HeLa cells. To further confer the importance of CLA-1 in SAA-binding and subsequent internalization, Alexa 488-SAA and CLA-1 are colocalized utilizing an anti-CLA-1 antibody (NOVUS NB-101). The majority of SAA colocalized with the plasma membrane as well as in intracellular pools of CLA-1 (yellow). No visible staining is demonstrated in Alexa 488-SAA incubated mock transfected cells for both SAA and an anti-CLA-1 antibody supporting results demonstrating only low levels of CLA-1 expression in mock-transfected HeLa cell (Vishnyakova, T. G. et al. (2003) “BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780).

Sites of SAA transport by CLA-1. To determine the cellular compartments where internalized SAA accumulates, co-localization experiments using Bodipy-transferrin and Bodipy-ceramide are performed. The majority of Alexa 488-SAA enters the transferrin (endocytic)-recycling compartment (ERC) after rapid endocytosis. A significant colocalization of SAA is seen with transferrin, which contrasts with weaker colocalization and significant green/red signal segregation for ceramide staining, indicating that ERC rather than the Golgi complex is the primary site of SAA transport by CLA-1. Very weak and scattered staining with Alexa 488-SAA is observed in mock-transfected HeLa cells.

SAA is transported to lysosomal compartments. To demonstrate that internalized SAA is partially transported to lysosomal compartments, a colocalization of Alexa 488-SAA and Lysotracker positive compartments is analyzed. Significant amounts of SAA are found to colocalize with the Lysotracker signal indicating that the initial binding to CLA-1 was also followed by SAA-transport to the lysosomal compartments.

Metabolism of SAA by CLA-1 overexpressing HeLa cells. Incomplete SAA degradation followed by a recycling of N-terminal SAA peptides to the macrophage plasma membrane has been suggested as a major factor in SAA accumulation and AA fibril deposition in amyloidosis (Kluve-Beckerman, B. et al. (2002) “A PULSE-CHASE STUDY TRACKING THE CONVERSION OF MACROPHAGE-ENDOCYTOSED SERUM AMYLOID A INTO EXTRACELLULAR AMYLOID,” Arthritis Rheum. 46:1905-1913; Elliott-Bryant, R. et al. (1998) “CATABOLISM OF LIPID-FREE RECOMBINANT APOLIPOPROTEIN SERUM AMYLOID A BY MOUSE MACROPHAGES IN VITRO RESULTS IN REMOVAL OF THE AMYLOID FIBRIL-FORMING AMINO TERMINUS,” Scand. J. Immunol. 48:241-247). To investigate the role of CLA-1 in SAA degradation, CLA-1 overexpressing HeLa cells are incubated with Alexa 488-SAA followed by SDS-PAGE and native PAGE electrophoresis of the cell lysates and conditioned media. Alexa 488-SAA is mostly found as a 12 kDa molecular mass band with small amounts of degradation products in extracts from CLA-1 overexpressing cells. Significantly lower levels of SAA (12 kDa molecular mass band) accumulation can be detected in mock-transfected cells. In contrast, media conditioned by CLA-1 overexpressing HeLa cells contained degraded SAA, which is seen as a number of lower molecular weight peptides in the range between 6-10 kDa. With increased time the amount of degraded products is elevated in conditioned media. Neither significant changes in SAA levels (12 kDa band) nor an accumulation of degraded products are observed in mock-transfected HeLa cells. Importantly, when associated with HDL, SAA was taken up and degraded to a lesser degree when compared to lipoprotein-free SAA indicating a protective effect of HDL. In addition, little or no degraded HDL apolipoproteins is observed in culture media upon the incubation of Alexa 488-HDL or Alexa 488-HDL/SAA complex with CLA-1 overexpressing HeLa cells.

### Conclusions

Serum Amyloid A is an acute phase plasma protein with unknown physiological function, which plays a central role in the development and progression of amyloidosis (Uhlar, C. M. & Whitehead, A. S. (1999) “SERUM AMYLOID A, THE MAJOR VERTEBRATE ACUTE-PHASE REACTANT,” Eur. J. Biochem. 265:501-523). In addition to a recent demonstration that the G protein-coupled receptor, FPRL1/LXA4R binds SAA followed by an induction of Ca2+ release and activation of the mitogen-activated protein kinases ERK1/2 and p38 (He, R. et al. (2003) “SERUM AMYLOID A INDUCES IL-8 SECRETION THROUGH A G PROTEIN-COUPLED RECEPTOR, FPRL1/LXA4R,” Blood 101:1572-1581), the existence of another SAA-receptor involved with SAA metabolism and degradation was suggested (Uhlar, C. M. & Whitehead, A. S. (1999) “SERUM AMYLOID A, THE MAJOR VERTEBRATE ACUTE-PHASE REACTANT,” Eur. J. Biochem. 265:501-523). However, no data have been reported on an involvement of the human HDL-receptor, CLA-1, in the cellular transport and degradation of SAA. The above-described data demonstrate that the uptake, which includes SAA binding and subsequent internalization, is dramatically increased in CLA-1 overexpressing HeLa cells. CLA-1 ligands as well as cold SAA competed against Alexa 488-SAA, indicating that SAA is an efficient ligand for the human HDL-receptor.

The observation of CLA-1 involvement with SAA intracellular transport is further confirmed by confocal microscopy experiments which indicate that after initial binding to the plasma membrane, lipoprotein-free SAA is transported to the endoplasmic recycling compartment, ERC, (transferrin recycling compartment). It has been recently reported that several CLA-1 ligands including apoA-I and LPS enter the Golgi complex while the association with HDL may redirect them to the ERC (Vishnyakova, T. G. et al. (2003) “BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780, Silver, D. L. et al. (2001) “HIGH DENSITY LIPOPROTEIN (HDL) PARTICLE UPTAKE MEDIATED BY SCAVENGER RECEPTOR CLASS B TYPE 1 RESULTS IN SELECTIVE SORTING OF HDL CHOLESTEROL FROM PROTEIN AND POLARIZED CHOLESTEROL SECRETION,” J. Biol. Chem. 276:25287-25293). The data demonstrate that in the time frame used, the majority of lipid-poor SAA is not efficiently transported to the Golgi complex while the possibility remains that with a longer period of time a larger portion of SAA can also accumulate in the Golgi. This observation has important applications. It has been shown that vesicular transport, which demonstrates a close association with CLA-1, leads to an initial accumulation of trackers in primary endosomes followed by subsequent trafficking to the ERC (Johannes, L. et al. (2002) “CLATHRIN-DEPENDENT OR NOT: IS IT STILL THE QUESTION?,” Traffic. 3, 443-451; Mallard, F. et al. (1998) “DIRECT PATHWAY FROM EARLY/RECYCLING ENDOSOMES TO THE GOLGI APPARATUS REVEALED THROUGH THE STUDY OF SHIGA TOXIN B-FRAGMENT TRANSPORT,” J. Cell Biol. 143, 973-990). This represents a mechanism of molecular retroendocytosis, a process that had been reported for HDL a decade ago and was again recently demonstrated in rodent SR-BI/CLA-1 transfected cells and isolated hepatocytes (Silver, D. L. et al. (2001) “HIGH DENSITY LIPOPROTEIN (HDL) PARTICLE UPTAKE MEDIATED BY SCAVENGER RECEPTOR CLASS B TYPE 1 RESULTS IN SELECTIVE SORTING OF HDL CHOLESTEROL FROM PROTEIN AND POLARIZED CHOLESTEROL SECRETION,” J. Biol. Chem. 276:25287-25293; Schmitz, G. et al. (1985) “INTERACTION OF HIGH DENSITY LIPOPROTEINS WITH CHOLESTERYL ESTER-LADEN MACROPHAGES: BIOCHEMICAL AND MORPHOLOGICAL CHARACTERIZATION OF CELL SURFACE RECEPTOR BINDING, ENDOCYTOSIS AND RESECRETION OF HIGH DENSITY LIPOPROTEINS BY MACROPHAGES,” EMBO J. 4, 613-622). In contrast to HDL, which undergoes neither intensive lysosomal degradation (Vishnyakova, T. G. et al. (2003) “BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780; Silver, D. L. et al. (2001) “HIGH DENSITY LIPOPROTEIN (HDL) PARTICLE UPTAKE MEDIATED BY SCAVENGER RECEPTOR CLASS B TYPE 1 RESULTS IN SELECTIVE SORTING OF HDL CHOLESTEROL FROM PROTEIN AND POLARIZED CHOLESTEROL SECRETION,” J. Biol. Chem. 276:25287-25293) nor transportation to lysosomal compartments during retroendocytosis, SAA accumulated in lysosomal compartments as revealed by the LysoTracker-red colocalization experiment. Although only a small amount of degradation products was observed intracellularly, the majority of partially degraded SAA was secreted into the culture media, indicating that in contrast to HDL delivery to ERC followed by resecretion of intact HDL apolipoproteins, SAA recycling involved a rapid partial degradation after lysosomal delivery.

In vitro studies also demonstrated that SAA uptake is strongly associated with macrophages and requires retroendocytosis-like transport of the N-terminal SAA peptides followed by extracellular AA deposition (Kluve-Beckerman, B. et al. (2002) “A PULSE-CHASE STUDY TRACKING THE CONVERSION OF MACROPHAGE-ENDOCYTOSED SERUM AMYLOID A INTO EXTRACELLULAR AMYLOID,” Arthritis Rheum. 46:1905-1913; Elliott-Bryant, R. et al. (1998) “CATABOLISM OF LIPID-FREE RECOMBINANT APOLIPOPROTEIN SERUM AMYLOID A BY MOUSE MACROPHAGES IN VITRO RESULTS IN REMOVAL OF THE AMYLOID FIBRIL-FORMING AMINO TERMINUS,” Scand. J. Immunol. 48:241-247; Kluve-Beckerman, B. et al. (2001) “BINDING, TRAFFICKING AND ACCUMULATION OF SERUM AMYLOID A IN PERITONEAL MACROPHAGES,” Scand. J. Immunol. 53:393-400). The data suggest a potentially important pathophysiological role for the human HDL-receptor, CLA-1, which may direct SAA such that SAA N-terminal peptides are produced which are required for the initial deposition of fibril amyloid as well as subsequent fibril development. Importantly, SAA-association with HDL strongly reduced SAA-uptake and was associated with a lower amount of degradation, suggesting a potentially protective effect of HDL against rapid SAA partial degradation as well as Amyloid A deposition.

Several mutated forms of apoA-I have been associated with an accumulation of amyloid deposits which contain predominantly mutated apoA-I (Zech, L. A. et al. (1983) “CHANGES IN PLASMA CHOLESTEROL AND TRIGLYCERIDE LEVELS AFTER TREATMENT WITH ORAL ISOTRETINOIN. A PROSPECTIVE STUDY,” Arch. Dermatol. 119, 987-993; Zech, L. A. et al. (1983) “METABOLISM OF HUMAN APOLIPOPROTEINS A-I AND A-II: COMPARTMENTAL MODELS,” J. Lipid Res. 24, 60-71; Obici, L. et al. (1999) “THE NEW APOLIPOPROTEIN A-I VARIANT LEU(174)->SER CAUSES HEREDITARY CARDIAC AMYLOIDOSIS, AND THE AMYLOID FIBRILS ARE CONSTITUTED BY THE 93-RESIDUE N-TERMINAL POLYPEPTIDE,” Am. J. Pathol. 155, 695-702). Such mutated apoA-I's may be delivered to transferrin dependent and lysosomal compartments as we observed with SAA instead of to the Golgi apparatus where normal apoA-I is transported (Vishnyakova, T. G. et al. (2003) “BINDING AND INTERNALIZATION OF LIPOPOLYSACCHARIDE BY CLA-1, A HUMAN ORTHOLOGUE OF RODENT SCAVENGER RECEPTOR B1,” J. Biol. Chem 278:22771-22780).

In summary, CLA-1 was demonstrated for the first time to function as an SAA endocytic recycling receptor involved in SAA partial degradation. This observation suggests a pathogenic model whereby initiation and further development of amyloid can occur. Supporting this role is the observation that amyloid deposition and accumulation is strongly associated with organ macrophages, which express high levels of CLA-1. This knowledge provides new treatments of amyloidosis, including a potential utilization of amphipathic helical peptides, which target CLA-1 and could block SAA uptake.

All publications and patent documents mentioned in this specification are herein incorporated by reference to the same extent as if each individual publication or patent document was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method for the treatment of sepsis, inflammation or infection comprising providing to a recipient a physiologically effective amount of a pharmaceutical composition comprising a molecule that targets SR-BI/CLA-1.

2. The method of claim 1, wherein said method provides a treatment for sepsis.

3. The method of claim 1, wherein said method provides a treatment for inflammation.

4. The method of claim 1, wherein said method provides a treatment for infection.

5. The method of claim 1, wherein said molecule is a peptide or is a peptide composition having a peptide portion.

6. The method of claim 5, wherein said peptide or peptide composition effects LPS-uptake or LPS-stimulated cytokine production.

7. The method of claim 6, wherein said molecule is a peptide that binds to an anionic amphipathic α-helix of SR-BI/CLA-1.

8. The method of claim 7, wherein said peptide is composed solely of L-amino acid residues.

9. The method of claim 7, wherein said peptide is composed solely of D-amino acid residues.

10. The method of claim 5, wherein said molecule is a peptide composition and wherein said peptide portion of said peptide composition binds to an anionic amphipathic α-helix of SR-BI/CLA-1.

11. The method of claim 10, wherein said peptide portion of said peptide composition is composed solely of L-amino acid residues.

12. The method of claim 10, wherein said peptide portion of said peptide composition is composed solely of D-amino acid residues.

13. The method of claim 1, wherein said molecule is selected from the group consisting of a cholesterol absorption inhibitor, a viral fusion inhibitor, a negatively charged lipid that binds to CLA-1 with a Kd lower than 10⁻⁷ M; an anti-SR-BI/CLA-1 antibody, of fragment thereof that binds SR-BI/CLA-1, and a chemical substance that binds to SR-BI/CLA-1 with a Kd lower than 10⁻⁷ M.

14. A pharmaceutical composition for the treatment of sepsis, inflammation or infection comprising providing to a recipient a physiologically effective amount of a pharmaceutical composition comprising:
(A) a molecule that targets SR-BI/CLA-1; and
(B) an auxiliary agent, excipient, or uptake facilitating agent.

15. The pharmaceutical composition of claim 14, wherein said physiologically effective amount is effective for providing a treatment for sepsis.

16. The pharmaceutical composition of claim 14, wherein said physiologically effective amount is effective for providing a treatment inflammation.

17. The pharmaceutical composition of claim 14, wherein said physiologically effective amount is effective for providing a treatment infection.

18. The pharmaceutical composition of claim 14, wherein said molecule is a peptide or is a peptide composition having a peptide portion.

19. The pharmaceutical composition of claim 18, wherein said peptide or peptide composition effects LPS-uptake or LPS-stimulated cytokine production.

20. The pharmaceutical composition of claim 18, wherein said molecule is a peptide that binds to an anionic amphipathic α-helix of SR-BI/CLA-1.

21. The pharmaceutical composition of claim 19, wherein said peptide is composed solely of L-amino acid residues.

22. The pharmaceutical composition of claim 19, wherein said peptide is composed solely of D-amino acid residues.

23. The pharmaceutical composition of claim 18, wherein said molecule is a peptide composition and wherein said peptide portion of said peptide composition binds to an anionic amphipathic α-helix of SR-BI/CLA-1.

24. The pharmaceutical composition of claim 23, wherein said peptide portion of said peptide composition is composed solely of L-amino acid residues.

25. The pharmaceutical composition of claim 23, wherein said peptide portion of said peptide composition is composed solely of D-amino acid residues.

26. The pharmaceutical composition of claim 14, wherein said molecule is selected from the group consisting of a cholesterol absorption inhibitor, a viral fusion inhibitor, a negatively charged lipid that binds to CLA-1 with a Kd lower than 10⁻⁷ M; an anti-SR-BI/CLA-1 antibody, of fragment thereof that binds SR-BI/CLA-1, and a chemical substance that binds to SR-BI/CLA-1 with a Kd lower than 10⁻⁷ M.


SEQUENCE LISTING (14 sequences)

SEQ ID NO: 1 - DNA, 21 bp, Homo sapiens
gtcttcaccaccatggagaag

SEQ ID NO: 2 - DNA, 25 bp, Homo sapiens
gcttcaccaccttcttgatgtcatc

SEQ ID NO: 3 - DNA, 21 bp, Homo sapiens
ccacccaacgaaggcttctgc

SEQ ID NO: 4 - DNA, 19 bp, Homo sapiens
ctgaatggcctccttatcc

SEQ ID NO: 5 - DNA, 20 bp, Homo sapiens
caactacaaagccctctttg

SEQ ID NO: 6 - DNA, 20 bp, Homo sapiens
cttggctgttctccatgaag

SEQ ID NO: 7 - DNA, 18 bp, Homo sapiens
ctgaaagctctccacctc

SEQ ID NO: 8 - DNA, 18 bp, Homo sapiens
gtgctgatgtaccagttg

SEQ ID NO: 9 - protein, 18 aa, Artificial Sequence
  Synthetic construct
DWLKAFYDKVAEKLKEAF

SEQ ID NO: 10 - protein, 37 aa, Artificial Sequence
  Synthetic construct
DWLKAFYDKVAEKLKEAFPDWLKAFYDKVAEKLKEAF

SEQ ID NO: 11 - protein, 37 aa, Artificial Sequence; identical to SEQ ID NO: 10
  Synthetic construct

SEQ ID NO: 12 - protein, 37 aa, Artificial Sequence; identical to SEQ ID NO: 10
  Synthetic construct

SEQ ID NO: 13 - protein, 37 aa, Artificial Sequence; identical to SEQ ID NO: 10
  Synthetic construct

SEQ ID NO: 14 - protein, 37 aa, Artificial Sequence; identical to SEQ ID NO: 10
  Synthetic construct